SEQUENCE LISTING (100 sequences)

SEQ ID NO: 1 - protein, 115 aa, Homo sapiens
MIPAKDMAKVMIVMLAICFLTKSDGKSVKKRSVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 2 - protein, 84 aa, Homo sapiens
SVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 3 - protein, 43 aa, Homo sapiens
MAHVRGLQLPGCLALAALCSLVHSQHVFLAPQQARSLLQRVRR

SEQ ID NO: 4 - protein, 20 aa, Homo sapiens
MYKMQLLSCIALTLVLVANS

SEQ ID NO: 5 - protein, 31 aa, Homo sapiens
MIPAKDMAKVMIVMLAICFLTKSDGKSVKKR

SEQ ID NO: 6 - protein, 229 aa, Homo sapiens
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 7 - protein, 226 aa, Homo sapiens
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 224 aa, Macaca mulatta
PPCPPCPAPELLGGPSVFLFPPKPKDTLMISRTPEVTCVVVDVSQEDPEVQFNWYVDGVEVHNAQTKPRERQFNSTYRVVSVLTVTHQDWLNGKEYTCKVSNKGLPAPIEKTISKAKGQPREPQVYILPPPQEELTKNQVSLTCLVTGFYPSDIAVEWESNGQPENTYKTTPPVLDSDGSYLLYSKLTVNKSRWQPGNIFTCSVMHEALHNHYTQKSLSVSPGK

SEQ ID NO: 9 - protein, 584 aa, Homo sapiens
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALG

SEQ ID NO: 10 - protein, 15 aa, synthetic construct
  REGION 1..15: note = synthetic construct
GGGGSGGGGSGGGGS

SEQ ID NO: 11 - protein, 322 aa, synthetic construct
  REGION 1..322: note = synthetic construct
MAHVRGLQLPGCLALAALCSLVHSQHVFLAPQQARSLLQRVRRSVSEIQLMHNLGKHLNSMERVEWLRKKLQDVHNGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 12 - DNA, 972 bp, synthetic construct
  misc_feature 1..972: note = synthetic construct
  source 1..972: mol_type = other DNA
atggctcacgttcgaggactgcagctgcctggatgtctggctcttgccgctctgtgtagcctggtgcacagccagcacgtgtttctggctcctcagcaagccagatcactgctgcagcgcgtcagaagaagcgtcagcgagatccagctgatgcacaacctgggcaagcacctgaacagcatggaacgcgtggagtggctgcggaagaaactgcaggacgtgcacaacggtggcggcggaggcggaagcggtggcggaggatcaggcggtggtggatctgccgagtctaagtacggacctccttgtcctccctgtcctgctcccgaagctgctggcggcccatccgtgtttctgttccctccaaagcctaaggacaccctgatgatcagcagaacccctgaagtgacctgcgtggtggtggacgtgtcccaagaggatcctgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtcgagtgggagagcaacggccagcctgagaacaactacaagaccacacctcctgtgctggacagcgacggctcattcttcctgtacagccggctgaccgtggacaagagcaggtggcaagagggcaacgtgttcagctgctccgtgatgcacgaggccctgcacaaccactacacccagaagtctctgagcctgagcctgggctgatga

SEQ ID NO: 13 - protein, 363 aa, synthetic construct
  REGION 1..363: note = synthetic construct
MAHVRGLQLPGCLALAALCSLVHSQHVFLAPQQARSLLQRVRRSVSEIQLMHNLGKHLNSMERVEWLRKKLQDVHNFVALGAPLAPRDAGSQRPRKKEDNVLVESHEKSLGEADKADGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 14 - DNA, 1095 bp, synthetic construct
  misc_feature 1..1095: note = synthetic construct
  source 1..1095: mol_type = other DNA
atggctcacgttcgaggactgcagctgcctggatgtctggctcttgccgctctgtgtagcctggtgcacagccagcacgtgtttctggctcctcagcaagccagatcactgctgcagcgcgtcagaagaagcgtcagcgagatccagctgatgcacaacctgggcaagcacctgaacagcatggaacgcgtggagtggctgcggaagaaactgcaggacgtgcacaacttcgtggccctgggagcacctctggctccaagagacgccggcagccaacggcctagaaagaaagaggacaacgtcctggtcgagagccacgagaagtctctgggcgaagccgataaggctgacggtggcggaggcggaggatctggtggcggtggttctggcggtggcggatctgccgagtctaagtacggacctccttgtcctccctgtcctgctcccgaagctgctggcggcccatccgtgtttctgttccctccaaagcctaaggacaccctgatgatcagcagaacccctgaagtgacctgcgtggtggtggacgtgtcccaagaggatcctgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtcgagtgggagagcaacggccagcctgagaacaactacaagaccacacctcctgtgctggacagcgacggctcattcttcctgtacagccggctgaccgtggacaagagccggtggcaagagggcaacgtgttcagctgctccgtgatgcacgaggccctgcacaaccactacacccagaaaagcctgtctctgagcctgggctgatga

SEQ ID NO: 15 - protein, 373 aa, synthetic construct
  REGION 1..373: note = synthetic construct
MAHVRGLQLPGCLALAALCSLVHSQHVFLAPQQARSLLQRVRRSVSEIQLMHNLGKHLNSMERVEWLRKKLQDVHNFVALGAPLAPRDAGSQRPRKKEDNVLVESHEKSLGEADKADVNVLTKAKSQGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 16 - DNA, 1125 bp, synthetic construct
  misc_feature 1..1125: note = synthetic construct
  source 1..1125: mol_type = other DNA
atggctcacgttcgaggactgcagctgcctggatgtctggctcttgccgctctgtgtagcctggtgcacagccagcacgtgtttctggctcctcagcaagccagatcactgctgcagcgcgtcagaagaagcgtcagcgagatccagctgatgcacaacctgggcaagcacctgaacagcatggaacgcgtggagtggctgcggaagaaactgcaggacgtgcacaacttcgtggccctgggagcacctctggctccaagagacgccggcagccaacggcctagaaagaaagaggacaacgtcctggtcgagagccacgagaagtctctgggcgaagccgataaggccgacgtgaacgtgctgaccaaggccaagtctcaaggtggcggaggcggaggatctggtggtggtggatctggcggcggaggttctgccgagtctaagtacggacctccttgtcctccctgtcctgctcccgaagctgctggcggcccatccgtgtttctgttccctccaaagcctaaggacaccctgatgatcagcagaacccctgaagtgacctgcgtggtggtcgacgtgtcccaagaggatcctgaggtgcagttcaattggtacgtggacggcgtggaagtgcacaacgccaagaccaagcctagagaggaacagttcaacagcacctacagagtggtgtctgtgctgaccgtgctgcaccaggattggctgaacggcaaagagtacaagtgcaaggtgtccaacaagggcctgcctagcagcatcgagaaaaccatctctaaggccaagggccagcctcgcgaaccccaggtttacacactgcctccaagccaagaggaaatgaccaagaaccaggtgtccctgacctgcctggtcaagggcttctacccttccgatatcgccgtcgagtgggagagcaacggccagcctgagaacaactacaagaccacacctcctgtgctggacagcgacggctcattcttcctgtacagccggctgaccgtggacaagagcaggtggcaagagggcaacgtgttcagctgctccgtgatgcacgaggccctgcacaaccactacacccagaaaagcctgtctctgagcctgggctgatga

SEQ ID NO: 17 - protein, 291 aa, synthetic construct
  REGION 1..291: note = synthetic construct
MIPAKDMAKVMIVMLAICFLTKSDGKSVKKRSVSEIQLMHNLGKHLNSMERVEWLRKKLQDVHNFDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - DNA, 876 bp, synthetic construct
  misc_feature 1..876: note = synthetic construct
  source 1..876: mol_type = other DNA
atgattcccgccaaggacatggccaaagtgatgatcgtgatgctggccatctgctttctgaccaagagcgacggcaagagcgtgaagaaaagaagcgtgtccgagatccagctgatgcacaacctgggcaagcacctgaacagcatggaacgcgtggagtggctgcggaagaaactgcaggacgtgcacaacttcgacaagacccacacctgtcctccctgtcctgctcccgaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgacaaagaaccaggtgtccctgacctgcctggtcaagggcttttaccccagcgacattgccgtcgagtgggagtctaacggccagcctgagaacaactacaagacaacccctcctgtgctggactccgacggctcattcttcctgtacagcaagctgacagtggacaagagccggtggcagcagggcaatgtgttcagctgtagcgtgatgcacgaggccctgcacaaccactacacccagaagtccctgagcctgtctcctggctga

SEQ ID NO: 19 - protein, 330 aa, synthetic construct
  REGION 1..330: note = synthetic construct
MIPAKDMAKVMIVMLAICFLTKSDGKSVKKRSVSEIQLMHNLGKHLNSMERVEWLRKKLQDVHNFVALGAPLAPRDAGSQRPRKKEDNVLVESHEKSLGEADKADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - DNA, 993 bp, synthetic construct
  misc_feature 1..993: note = synthetic construct
  source 1..993: mol_type = other DNA
atgattcccgccaaggacatggccaaagtgatgatcgtgatgctggccatctgctttctgaccaagagcgacggcaagagcgtgaagaaaagaagcgtgtccgagatccagctgatgcacaacctgggcaagcacctgaacagcatggaacgcgtggagtggctgcggaagaaactgcaggacgtgcacaacttcgtggccctgggagcacctctggctcctagggacgccggcagccagcggcctagaaagaaagaggataacgtcctggtcgagagccacgagaagtctctgggcgaagccgacaaggccgacaaaacacacacctgtcctccctgtcctgctcccgaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtccacaacgccaagaccaagcctagagaggaacagtacaacagcacctacagagtggtgtccgtgctgaccgtgctgcaccaggattggctgaacggcaaagagtacaagtgcaaggtgtctaacaaggccctgcctgctcctatcgagaaaaccatcagcaaggccaagggccagcctagggaaccccaggtttacacactccctccaagcagggacgagctgacaaagaaccaggtgtccctgacctgcctggtcaagggcttctacccttccgatatcgccgtcgagtgggagtccaacggccagccagagaacaactacaagacaacccctcctgtgctggactccgacggctcattcttcctgtacagcaagctgacagtggacaagagccggtggcagcagggcaatgtgttcagctgtagcgtgatgcacgaggccctgcacaaccactacacccagaagtccctgagcctgtctcctggctga

SEQ ID NO: 21 - protein, 331 aa, synthetic construct
  REGION 1..331: note = synthetic construct
MIPAKDMAKVMIVMLAICFLTKSDGKSVKKRSVSEIQLMHNLGKHLNSMERVEWLRKKLQDVHNFVALGAPLAPRDAGSQRPRKKEDNVLVESHEKSLGEADKADDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - DNA, 996 bp, synthetic construct
  misc_feature 1..996: note = synthetic construct
  source 1..996: mol_type = other DNA
atgattcccgccaaggacatggccaaagtgatgatcgtgatgctggccatctgctttctgaccaagagcgacggcaagagcgtgaagaaaagaagcgtgtccgagatccagctgatgcacaacctgggcaagcacctgaacagcatggaacgcgtggagtggctgcggaagaaactgcaggacgtgcacaacttcgtggccctgggagcacctctggctcctagagacgccggcagccagcggcctagaaagaaagaggataacgtcctggtcgagagccacgagaagtctctgggcgaagccgacaaggccgacgacaagacacacacctgtcctccctgtcctgctcccgaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgacaaagaaccaggtgtccctgacctgcctggtcaagggcttctacccttccgatatcgccgtcgagtgggagtccaacggccagccagagaacaactacaagacaacccctcctgtgctggactccgacggctcattcttcctgtacagcaagctgacagtggacaagagccggtggcagcagggcaatgtgttcagctgtagcgtgatgcacgaggccctgcacaaccactacacccagaagtccctgagcctgtctcctggctga

SEQ ID NO: 23 - protein, 332 aa, synthetic construct
  REGION 1..332: note = synthetic construct
MIPAKDMAKVMIVMLAICFLTKSDGKSVKKRSVSEIQLMHNLGKHLNSMERVEWLRKKLQDVHNFVALGAPLAPRDAGSQRPRKKEDNVLVESHEKSLGEADKADVDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - DNA, 999 bp, synthetic construct
  misc_feature 1..999: note = synthetic construct
  source 1..999: mol_type = other DNA
atgattcccgccaaggacatggccaaagtgatgatcgtgatgctggccatctgctttctgaccaagagcgacggcaagagcgtgaagaaaagaagcgtgtccgagatccagctgatgcacaacctgggcaagcacctgaacagcatggaacgcgtggagtggctgcggaagaaactgcaggacgtgcacaacttcgtggccctgggagcacctctggctcctagagacgccggcagccagcggcctagaaagaaagaggataacgtcctggtcgagagccacgagaagtctctgggcgaagccgacaaggccgacgtggacaagacacacacctgtcctccctgtcctgctcccgaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgacaaagaaccaggtgtccctgacctgcctggtcaagggcttctacccttccgatatcgccgtcgagtgggagtccaacggccagccagagaacaactacaagacaacccctcctgtgctggactccgacggctcattcttcctgtacagcaagctgacagtggacaagagccggtggcagcagggcaatgtgttcagctgtagcgtgatgcacgaggccctgcacaaccactacacccagaagtccctgagcctgtctcctggctga

SEQ ID NO: 25 - protein, 677 aa, synthetic construct
  REGION 1..677: note = synthetic construct
MAHVRGLQLPGCLALAALCSLVHSQHVFLAPQQARSLLQRVRRSVSEIQLMHNLGKHLNSMERVEWLRKKLQDVHNGGGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALG

SEQ ID NO: 26 - DNA, 2037 bp, synthetic construct
  misc_feature 1..2037: note = synthetic construct
  source 1..2037: mol_type = other DNA
atggctcacgttcgaggactgcagctgcctggatgtctggctcttgccgctctgtgtagcctggtgcacagccagcacgtgtttctggctcctcagcaagccagatcactgctgcagcgcgtcagaagaagcgtcagcgagatccagctgatgcacaacctgggcaagcacctgaacagcatggaacgcgtggagtggctgcggaagaaactgcaggacgtgcacaacggtggcggcggaggcggaagcggtggcggaggatcaggcggtggtggatctgacgcccacaagtctgaagtggcccaccggttcaaggacctgggcgaagagaatttcaaggccctggtgctgatcgccttcgctcagtacctgcagcagtgccccttcgaggaccacgtgaagctggtcaacgaagtgaccgagttcgccaagacctgcgtggccgacgagagcgccgagaactgtgataagagcctgcacaccctgttcggcgacaagctgtgtacagtggccacactgagagaaacctacggcgagatggccgactgctgcgccaagcaagagcccgagagaaacgagtgcttcctgcagcacaaggacgacaaccccaacctgcctagactcgtgcggcctgaagtggacgtgatgtgcaccgccttccacgacaacgaggaaaccttcctgaagaagtacctgtacgagatcgccagacggcacccctacttttacgcccctgagctgctgttcttcgccaagcggtataaggccgccttcaccgagtgttgtcaggccgctgataaggctgcctgcctgctgcctaagctggacgagcttagagacgagggcaaagccagctccgccaagcagagactgaagtgtgccagcctgcagaagttcggcgagagagcctttaaggcctgggccgttgctagactgagccagagatttcccaaggccgagtttgccgaggtgtccaagctcgtgaccgacctgacaaaggtgcacaccgagtgctgccacggcgacctgctggaatgcgccgacgatagagccgacctggccaagtacatctgcgagaaccaggacagcatcagcagcaagctgaaagagtgctgcgagaagcctctgctggaaaagagccactgtatcgccgaggtggaaaacgacgagatgcccgccgatctgccttctctggccgccgattttgtggaaagcaaggacgtgtgcaagaactacgccgaggccaaggacgtgttcctgggcatgtttctgtacgagtacgcccgcagacaccccgactactctgttgtgctgctgctgagactggccaaaacctacgagacaaccctggaaaagtgctgtgccgccgctgatcctcacgagtgttacgccaaggtgttcgacgagttcaagccactggtggaagaaccccagaacctgatcaagcagaactgcgagctgttcgagcagctgggcgagtacaagttccagaacgccctgctcgtgcggtacaccaagaaggtgccccaggtttccacacctacactggttgaggtgtcccggaatctgggcaaagtgggcagcaagtgctgcaagcaccctgaggccaagagaatgccttgcgccgaggattacctgagcgtggtgctgaatcagctgtgcgtgctgcacgagaaaacccctgtgtccgacagagtgaccaagtgctgtaccgagagcctggtcaacagacggccttgctttagcgccctcgaggtggacgagacatacgtgcccaaagagttcaacgccgagacattcaccttccacgccgacatctgtaccctgagcgagaaagagcggcagatcaagaaacagactgccctggtggaactggtcaagcacaagcccaaggccaccaaagaacagctgaaggccgtgatggacgacttcgccgccttcgtggaaaaatgctgcaaggccgacgacaaagagacatgcttcgccgaagagggcaagaaactggtggccgcttctcaggctgccctgggatgataa

SEQ ID NO: 27 - protein, 677 aa, synthetic construct; identical to SEQ ID NO: 25
  REGION 1..677: note = synthetic construct

SEQ ID NO: 28 - DNA, 2037 bp, synthetic construct; identical to SEQ ID NO: 26
  misc_feature 1..2037: note = synthetic construct
  source 1..2037: mol_type = other DNA

SEQ ID NO: 29 - protein, 728 aa, synthetic construct
  REGION 1..728: note = synthetic construct
MAHVRGLQLPGCLALAALCSLVHSQHVFLAPQQARSLLQRVRRSVSEIQLMHNLGKHLNSMERVEWLRKKLQDVHNFVALGAPLAPRDAGSQRPRKKEDNVLVESHEKSLGEADKADVNVLTKAKSQGGGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALG

SEQ ID NO: 30 - DNA, 2190 bp, synthetic construct
  misc_feature 1..2190: note = synthetic construct
  source 1..2190: mol_type = other DNA
atggctcacgttcgaggactgcagctgcctggatgtctggctcttgccgctctgtgtagcctggtgcacagccagcacgtgtttctggctcctcagcaagccagatcactgctgcagcgcgtcagaagaagcgtcagcgagatccagctgatgcacaacctgggcaagcacctgaacagcatggaacgcgtggagtggctgcggaagaaactgcaggacgtgcacaacttcgtggccctgggagcacctctggctccaagagacgccggcagccaacggcctagaaagaaagaggacaacgtcctggtcgagagccacgagaagtctctgggcgaagccgataaggccgacgtgaacgtgctgaccaaggccaagtctcaaggtggcggaggcggaggatctggtggtggtggatctggcggcggaggttctgacgcccacaaatctgaagtggcccaccggttcaaggacctgggagaagagaacttcaaggccctggtgctgatcgccttcgctcagtacctgcagcagtgccccttcgaggaccacgtgaagctggtcaacgaagtgaccgagttcgccaagacctgcgtggccgacgagagcgccgagaactgtgataagagcctgcacaccctgttcggcgacaagctgtgtacagtggccacactgagagaaacctacggcgagatggccgactgctgcgccaagcaagagcccgagagaaacgagtgcttcctgcagcacaaggacgacaaccccaacctgcctagactcgtgcggcctgaagtggacgtgatgtgcaccgccttccacgacaacgaggaaaccttcctgaagaagtacctgtacgagatcgccagacggcacccctacttttacgcccctgagctgctgttcttcgccaagcggtataaggccgccttcaccgagtgttgtcaggccgctgataaggctgcctgcctgctgcctaagctggacgagcttagagacgagggcaaagccagctccgccaagcagagactgaagtgtgccagcctgcagaagttcggcgagagagcctttaaggcctgggccgttgctagactgagccagagatttcccaaggccgagtttgccgaggtgtccaagctcgtgaccgacctgacaaaggtgcacaccgagtgctgccacggcgacctgctggaatgcgccgacgatagagccgacctggccaagtacatctgcgagaaccaggacagcatcagcagcaagctgaaagagtgctgcgagaagcctctgctggaaaagagccactgtatcgccgaggtggaaaacgacgagatgcccgccgatctgccttctctggccgccgattttgtggaaagcaaggacgtgtgcaagaactacgccgaggccaaggacgtgttcctgggcatgtttctgtacgagtacgcccgcagacaccccgactactctgttgtgctgctgctgagactggccaaaacctacgagacaaccctggaaaagtgctgtgccgccgctgatcctcacgagtgttacgccaaggtgttcgacgagttcaagccactggtggaagaaccccagaacctgatcaagcagaactgcgagctgttcgagcagctgggcgagtacaagttccagaacgccctgctcgtgcggtacaccaagaaggtgccccaggtttccacacctacactggttgaggtgtcccggaatctgggcaaagtgggcagcaagtgctgcaagcaccctgaggccaagagaatgccttgcgccgaggattacctgagcgtggtgctgaatcagctgtgcgtgctgcacgagaaaacccctgtgtccgacagagtgaccaagtgctgtaccgagagcctggtcaacagacggccttgctttagcgccctcgaggtggacgagacatacgtgcccaaagagttcaacgccgagacattcaccttccacgccgacatctgtaccctgagcgagaaagagcggcagatcaagaaacagactgccctggtggaactggtcaagcacaagcccaaggccaccaaagaacagctgaaggccgtgatggacgacttcgccgccttcgtggaaaaatgctgcaaggccgacgacaaagagacatgcttcgccgaagagggcaagaaactggtggccgcttctcaggctgccctgggatgataa

SEQ ID NO: 31 - DNA, 665 bp, synthetic construct
  misc_feature 1..665: note = synthetic construct
  source 1..665: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 32 - DNA, 529 bp, synthetic construct
  misc_feature 1..529: note = synthetic construct
  source 1..529: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatc

SEQ ID NO: 33 - DNA, 978 bp, synthetic construct
  misc_feature 1..978: note = synthetic construct
  source 1..978: mol_type = other DNA
ggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagaggcgttcaggtggaaaccatcagtccaggggatggccgaacttttccaaagagagggcagacttgcgtcgtgcattatactggtatgctggaggatgggaaaaagttcgactcttccagagatcggaacaaaccattcaaattcatgctcgggaaacaggaagttatccgcggatgggaggagggcgtggcccagatgtccgtgggccagcgcgccaagctaaccatctccccagactacgcctacggagccaccggacaccccggtatcatacccccacacgccacccttgtgtttgacgtggaactgcttaagctagagactagaggcgtgcaggtcgagaccatcagccccggcgacggccgcacctttcccaagagaggccagacttgcgtggtccactacaccggcatgctggaggacggcaagaagttcgacagcagccgcgaccgcaacaagcccttcaagttcatgctgggcaaacaggaagtgatccgcggctgggaggaaggcgtggctcagatgagcgtggggcagcgggccaagctgaccatcagccccgactatgcctacggcgccaccggccaccccggcatcatccccccccacgccaccctggtgttcgacgtggagctgctgaagctggagtga

SEQ ID NO: 34 - protein, 107 aa, synthetic construct
  REGION 1..107: note = synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 35 - DNA, 18 bp, synthetic construct
  misc_feature 1..18: note = synthetic construct
  source 1..18: mol_type = other DNA
aatgatgggcgctcgagt

SEQ ID NO: 36 - DNA, 121 bp, synthetic construct
  misc_feature 1..121: note = synthetic construct
  source 1..121: mol_type = other DNA
aacattttgacacccccataatatttttccagaattaacagtataaattgcatctcttgttcaagagttccctatcactctctttaatcactactcacagtaacctcaactcctgccacaa

SEQ ID NO: 37 - DNA, 2211 bp, Adeno-Associated Virus rh91
  source 1..2211: mol_type = unassigned DNA
atggctgctgacggttatcttccagattggctcgaggacaacctttctgaaggcattcgtgagtggtgggctctgaaacctggagcccctaaacccaaagcgaaccaacaaaagcaggacgacggccggggtcttgtgcttccgggttacaaatacctcggacccttcaacggactcgacaaaggagagccggtcaacgcggcggacgcggcagccctcgaacacgacaaagcttacgaccagcagctcaaggccggtgacaacccgtacctccggtacaaccacgccgacgccgagtttcaggagcgtcttcaagaagatacgtcttttgggggcaaccttggcagagcagtcttccaggccaaaaagagggttcttgagccttttggtctggttgaggaagcagctaaaacggctcctggaaagaagaggcctgtagagcagtctcctcaggaaccggactcatcatctggtattggcaaatcgggccagcagcctgccaaaaaaagactaaatttcggtcagactggcgactcagagtcagtccccgaccctcaacctctcggagaacctccagaaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggcccttcctacctacaacaaccacctctacaagcaaatctccagcgcttcaacgggggccagtaacgacaaccactactttggctacagcaccccctgggggtattttgatttcaacagattccactgccacttctcaccacgtgactggcagcgactcattaacaacaactggggattccggcccaagagactcaacttcaagctcttcaacatccaggtcaaggaggtcacgacgaatgatggcgtcacaaccatcgctaataaccttaccagcacggttcaagtgttctcggactcggagtaccagctgccgtacgtcctcggttctgcgcaccagggctgcctccctccgttcccggcggacgtattcatgattcctcagtatggatacctcaccctgaacaacggaagtcaagcggtgggacgctcatccttttactgcctggagtacttcccttcgcagatgctaaggactggaaataacttcaccttcagctataccttcgaggatgtaccttttcacagcagctacgctcacagccagagtttggatcgcttgatgaatcctcttattgatcagtatctgtactacctgaacagaacgcaaaatcaatctggaagtgcacaaaacaaggacctgctttttagccgggggtctcctgctggcatgtctgttcagcccaaaaattggctacctgggccctgctaccggcaacagagagtttcaaagactaaaacagacaacaacaacagtaactttacctggacaggtgccagcaaatataatctcaatggccgcgaatcgatcattaatccaggaaccgctatggccagtcacaaggacgatgaagacaaatttttccctatgagcggcgttatgatatttggcaaagaaaatgcaggagcaagtaacactgcattagataatgtaatgattacggatgaagaagagattaaagctaccaatcctgtggcaacagagagatttggaactgtggcagtcaacttgcagagctcaaatacagaccccgcaactggagacgtccatgtcatgggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcctcatcaaaaatactccggtaccggcaaatcctccggcagagttcagcgctacaaagtttgcttcatttatcactcagtactccactggacaggtcagcgtggaaattgagtgggagctacagaaagaaaacagcaaacgttggaatccagaggtgcagtacacttccaactacgcgaagtctgccaatgtggactttactgtagacaacaatggtctttatactgaacctcgccctattggaacccggtatctcacacgacccttgtaa

SEQ ID NO: 38 - protein, 736 aa, Adeno-Associated Virus rh91
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEAAKTAPGKKRPVEQSPQEPDSSSGIGKSGQQPAKKRLNFGQTGDSESVPDPQPLGEPPETPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKENAGASNTALDNVMITDEEEIKATNPVATERFGTVAVNLQSSNTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 39 - protein, 110 aa, synthetic construct
  REGION 1..110: note = synthetic construct
MYKMQLLSCIALTLVLVANSKSVKKRSVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 40 - DNA, 336 bp, synthetic construct
  misc_feature 1..336: note = synthetic construct
  source 1..336: mol_type = other DNA
atgtacaagatgcagctgctgagctgtatcgccctgacactggtgctggtggccaactccaagagcgtgaagaaaagaagcgtgtccgagatccagctgatgcacaacctgggcaagcacctgaacagcatggaacgcgtggagtggctgcggaagaaactgcaggacgtgcacaacttcgtggccctgggagcacctctggctcctagagacgccggcagccagcggcctagaaagaaagaggataacgtcctggtcgagagccacgagaagtctctgggcgaagccgataaggccgacgtgaacgtgctgaccaaggccaagagccagtgatga

SEQ ID NO: 41 - DNA, 351 bp, synthetic construct
  misc_feature 1..351: note = synthetic construct
  source 1..351: mol_type = other DNA
atgattcccgccaaggacatggccaaagtgatgatcgtgatgctggccatctgctttctgaccaagagcgacggcaagagcgtgaagaaaagaagcgtgtccgagatccagctgatgcacaacctgggcaagcacctgaacagcatggaacgcgtggagtggctgcggaagaaactgcaggacgtgcacaacttcgtggccctgggagcacctctggctcctagagacgccggcagccagcggcctagaaagaaagaggataacgtcctggtcgagagccacgagaagtctctgggcgaagccgataaggccgacgtgaacgtgctgaccaaggccaagagccagtgatga

SEQ ID NO: 42 - protein, 326 aa, synthetic construct
  REGION 1..326: note = synthetic construct
MIPAKDMAKVMIVMLAICFLTKSDGSVSEIQLMHNLGKHLNSMERVEWLRKKLQDVHNFVALGAPLAPRDAGSQRPRKKEDNVLVESHEKSLGEADKADVDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - DNA, 3312 bp, synthetic construct
  misc_feature 1..3312: note = synthetic construct
  source 1..3312: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcaagcttgccaccatgattcccgccaaggacatggccaaagtgatgatcgtgatgctggccatctgctttctgaccaagagcgacggcagcgtgtccgagattcagctgatgcacaacctgggcaagcacctgaacagcatggaacgcgtggagtggctgcggaagaaactgcaggacgtgcacaacttcgtggccctgggagcacctctggctcctagagacgccggcagccagcggcctagaaagaaagaggataacgtcctggtcgagagccacgagaagtctctgggcgaagccgacaaggccgacgtggacaagacacacacctgtcctccctgtcctgctcccgaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgacaaagaaccaggtgtccctgacctgcctggtcaagggcttctacccttccgatatcgccgtcgagtgggagtccaacggccagccagagaacaactacaagacaacccctcctgtgctggactccgatggctcattcttcctgtacagcaagctgacagtggacaagagccggtggcagcagggcaatgtgttcagctgtagcgtgatgcacgaggccctgcacaaccactacacccagaagtccctgagcctgtctcctggctgatgaggtacctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 44 - protein, 325 aa, synthetic construct
  REGION 1..325: note = synthetic construct
MIPAKDMAKVMIVMLAICFLTKSDGSVSEIQLMHNLGKHLNSMERVEWLRKKLQDVHNFVALGAPLAPRDAGSQRPRKKEDNVLVESHEKSLGEADKADDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - DNA, 3309 bp, synthetic construct
  misc_feature 1..3309: note = synthetic construct
  source 1..3309: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcaagcttgccaccatgattcccgccaaggacatggccaaagtgatgatcgtgatgctggccatctgctttctgaccaagagcgacggcagcgtgtccgagattcagctgatgcacaacctgggcaagcacctgaacagcatggaacgcgtggagtggctgcggaagaaactgcaggacgtgcacaacttcgtggccctgggagcacctctggctcctagagacgccggcagccagcggcctagaaagaaagaggataacgtcctggtcgagagccacgagaagtctctgggcgaagccgacaaggccgacgacaagacacacacctgtcctccctgtcctgctcccgaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgacaaagaaccaggtgtccctgacctgcctggtcaagggcttctacccttccgatatcgccgtcgagtgggagtccaacggccagccagagaacaactacaagacaacccctcctgtgctggactccgatggctcattcttcctgtacagcaagctgacagtggacaagagccggtggcagcagggcaatgtgttcagctgtagcgtgatgcacgaggccctgcacaaccactacacccagaagtccctgagcctgtctcctggctgatgaggtacctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 46 - protein, 324 aa, synthetic construct
  REGION 1..324: note = synthetic construct
MIPAKDMAKVMIVMLAICFLTKSDGSVSEIQLMHNLGKHLNSMERVEWLRKKLQDVHNFVALGAPLAPRDAGSQRPRKKEDNVLVESHEKSLGEADKADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - DNA, 975 bp, synthetic construct
  misc_feature 1..975: note = synthetic construct
  source 1..975: mol_type = other DNA
atgattcccgccaaggacatggccaaagtgatgatcgtgatgctggccatctgctttctgaccaagagcgacggcagcgtgtccgagattcagctgatgcacaacctgggcaagcacctgaacagcatggaacgcgtggagtggctgcggaagaaactgcaggacgtgcacaacttcgtggccctgggagcacctctggctcctagagacgccggcagccagcggcctagaaagaaagaggataacgtcctggtcgagagccacgagaagtctctgggcgaagccgacaaggccgacaagacacacacctgtcctccctgtcctgctcccgaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgacaaagaaccaggtgtccctgacctgcctggtcaagggcttctacccttccgatatcgccgtcgagtgggagtccaacggccagccagagaacaactacaagacaacccctcctgtgctggactccgatggctcattcttcctgtacagcaagctgacagtggacaagagccggtggcagcagggcaatgtgttcagctgtagcgtgatgcacgaggccctgcacaaccactacacccagaagtccctgagcctgtctcctggctga

SEQ ID NO: 48 - protein, 285 aa, synthetic construct
  REGION 1..285: note = synthetic construct
MIPAKDMAKVMIVMLAICFLTKSDGSVSEIQLMHNLGKHLNSMERVEWLRKKLQDVHNFDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - DNA, 857 bp, synthetic construct
  misc_feature 1..857: note = synthetic construct
  source 1..857: mol_type = other DNA
atgattcccgccaaggacatggccaaagtgatgatcgtgatgctggccatctgctttctgaccaagagcgacggcagcgtgtccgagattcagctgatgcacaacctgggcaagcacctgaacagcatggaacgcgtggagtggctgcggaagaaactgcaggacgtgcacaacttcgacaagacccacacctgtcctccctgtcctgctcccgaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgacaaagaaccaggtgtccctgacctgcctggtcaagggcttctacccttccgatatcgccgtcgagtgggagtccaacggccagccagagaacaactacaagacaacccctcctgtgctggactccgatggctcattcttcctgtacagcaagctgacagtggacaagagccggtggcagcagggcaatgtgttcagctgtagcgtgatgcacgaggccctgcacaaccactacacccagaagtccctgagcctgtctcctggctg

SEQ ID NO: 50 - DNA, 3189 bp, synthetic construct
  misc_feature 1..3189: note = synthetic construct
  source 1..3189: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcaagcttgccaccatgattcccgccaaggacatggccaaagtgatgatcgtgatgctggccatctgctttctgaccaagagcgacggcagcgtgtccgagattcagctgatgcacaacctgggcaagcacctgaacagcatggaacgcgtggagtggctgcggaagaaactgcaggacgtgcacaacttcgacaagacccacacctgtcctccctgtcctgctcccgaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgacaaagaaccaggtgtccctgacctgcctggtcaagggcttctacccttccgatatcgccgtcgagtgggagtccaacggccagccagagaacaactacaagacaacccctcctgtgctggactccgatggctcattcttcctgtacagcaagctgacagtggacaagagccggtggcagcagggcaatgtgttcagctgtagcgtgatgcacgaggccctgcacaaccactacacccagaagtccctgagcctgtctcctggctgatgaggtacctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 51 - DNA, 3333 bp, synthetic construct
  misc_feature 1..3333: note = synthetic construct
  source 1..3333: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccctgcagcgggaattgccaccatggctcacgttcgaggactgcagctgcctggatgtctggctcttgccgctctgtgtagcctggtgcacagccagcacgtgtttctggctcctcagcaagccagatcactgctgcagcgcgtcagaagaagcgtcagcgagatccagctgatgcacaacctgggcaagcacctgaacagcatggaacgcgtggagtggctgcggaagaaactgcaggacgtgcacaacggtggcggcggaggcggaagcggtggcggaggatcaggcggtggtggatctgccgagtctaagtacggacctccttgtcctccctgtcctgctcccgaagctgctggcggcccatccgtgtttctgttccctccaaagcctaaggacaccctgatgatcagcagaacccctgaagtgacctgcgtggtggtggacgtgtcccaagaggatcctgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtcgagtgggagagcaacggccagcctgagaacaactacaagaccacacctcctgtgctggacagcgacggctcattcttcctgtacagccggctgaccgtggacaagagcaggtggcaagagggcaacgtgttcagctgctccgtgatgcacgaggccctgcacaaccactacacccagaagtctctgagcctgagcctgggctgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 52 - DNA, 3207 bp, synthetic construct
  misc_feature 1..3207: note = synthetic construct
  source 1..3207: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcaagcttgccaccatgattcccgccaaggacatggccaaagtgatgatcgtgatgctggccatctgctttctgaccaagagcgacggcaagagcgtgaagaaaagaagcgtgtccgagatccagctgatgcacaacctgggcaagcacctgaacagcatggaacgcgtggagtggctgcggaagaaactgcaggacgtgcacaacttcgacaagacccacacctgtcctccctgtcctgctcccgaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgacaaagaaccaggtgtccctgacctgcctggtcaagggcttttaccccagcgacattgccgtcgagtgggagtctaacggccagcctgagaacaactacaagacaacccctcctgtgctggactccgacggctcattcttcctgtacagcaagctgacagtggacaagagccggtggcagcagggcaatgtgttcagctgtagcgtgatgcacgaggccctgcacaaccactacacccagaagtccctgagcctgtctcctggctgatgaggtacctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 53 - DNA, 4398 bp, synthetic construct
  misc_feature 1..4398: note = synthetic construct
  source 1..4398: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccctgcagcgggaattgccaccatggctcacgttcgaggactgcagctgcctggatgtctggctcttgccgctctgtgtagcctggtgcacagccagcacgtgtttctggctcctcagcaagccagatcactgctgcagcgcgtcagaagaagcgtcagcgagatccagctgatgcacaacctgggcaagcacctgaacagcatggaacgcgtggagtggctgcggaagaaactgcaggacgtgcacaacggtggcggcggaggcggaagcggtggcggaggatcaggcggtggtggatctgacgcccacaagtctgaagtggcccaccggttcaaggacctgggcgaagagaatttcaaggccctggtgctgatcgccttcgctcagtacctgcagcagtgccccttcgaggaccacgtgaagctggtcaacgaagtgaccgagttcgccaagacctgcgtggccgacgagagcgccgagaactgtgataagagcctgcacaccctgttcggcgacaagctgtgtacagtggccacactgagagaaacctacggcgagatggccgactgctgcgccaagcaagagcccgagagaaacgagtgcttcctgcagcacaaggacgacaaccccaacctgcctagactcgtgcggcctgaagtggacgtgatgtgcaccgccttccacgacaacgaggaaaccttcctgaagaagtacctgtacgagatcgccagacggcacccctacttttacgcccctgagctgctgttcttcgccaagcggtataaggccgccttcaccgagtgttgtcaggccgctgataaggctgcctgcctgctgcctaagctggacgagcttagagacgagggcaaagccagctccgccaagcagagactgaagtgtgccagcctgcagaagttcggcgagagagcctttaaggcctgggccgttgctagactgagccagagatttcccaaggccgagtttgccgaggtgtccaagctcgtgaccgacctgacaaaggtgcacaccgagtgctgccacggcgacctgctggaatgcgccgacgatagagccgacctggccaagtacatctgcgagaaccaggacagcatcagcagcaagctgaaagagtgctgcgagaagcctctgctggaaaagagccactgtatcgccgaggtggaaaacgacgagatgcccgccgatctgccttctctggccgccgattttgtggaaagcaaggacgtgtgcaagaactacgccgaggccaaggacgtgttcctgggcatgtttctgtacgagtacgcccgcagacaccccgactactctgttgtgctgctgctgagactggccaaaacctacgagacaaccctggaaaagtgctgtgccgccgctgatcctcacgagtgttacgccaaggtgttcgacgagttcaagccactggtggaagaaccccagaacctgatcaagcagaactgcgagctgttcgagcagctgggcgagtacaagttccagaacgccctgctcgtgcggtacaccaagaaggtgccccaggtttccacacctacactggttgaggtgtcccggaatctgggcaaagtgggcagcaagtgctgcaagcaccctgaggccaagagaatgccttgcgccgaggattacctgagcgtggtgctgaatcagctgtgcgtgctgcacgagaaaacccctgtgtccgacagagtgaccaagtgctgtaccgagagcctggtcaacagacggccttgctttagcgccctcgaggtggacgagacatacgtgcccaaagagttcaacgccgagacattcaccttccacgccgacatctgtaccctgagcgagaaagagcggcagatcaagaaacagactgccctggtggaactggtcaagcacaagcccaaggccaccaaagaacagctgaaggccgtgatggacgacttcgccgccttcgtggaaaaatgctgcaaggccgacgacaaagagacatgcttcgccgaagagggcaagaaactggtggccgcttctcaggctgccctgggatgataaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 54 - DNA, 3611 bp, synthetic construct
  misc_feature 1..3611: note = synthetic construct
  source 1..3611: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagtacgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattcgccctgcagcgggaattgccaccatggctcacgttcgaggactgcagctgcctggatgtctggctcttgccgctctgtgtagcctggtgcacagccagcacgtgtttctggctcctcagcaagccagatcactgctgcagcgcgtcagaagaagcgtcagcgagatccagctgatgcacaacctgggcaagcacctgaacagcatggaacgcgtggagtggctgcggaagaaactgcaggacgtgcacaacggtggcggcggaggcggaagcggtggcggaggatcaggcggtggtggatctgccgagtctaagtacggacctccttgtcctccctgtcctgctcccgaagctgctggcggcccatccgtgtttctgttccctccaaagcctaaggacaccctgatgatcagcagaacccctgaagtgacctgcgtggtggtggacgtgtcccaagaggatcctgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtcgagtgggagagcaacggccagcctgagaacaactacaagaccacacctcctgtgctggacagcgacggctcattcttcctgtacagccggctgaccgtggacaagagcaggtggcaagagggcaacgtgttcagctgctccgtgatgcacgaggccctgcacaaccactacacccagaagtctctgagcctgagcctgggctgatgaggtaccgatctttttccctctgccaaaaattatggggacatcatgaagccccttgagcatctgacttctggctaataaaggaaatttattttcattgcaatagtgtgttggaattttttgtgtctctcactcggaaggcgcgaccggttgggcatggccaggtagcctatgctgtgtctggacgtcctcctgctggtatagttattttaaaatcagaaggacagggaagggagcagtggttcacgcctgtaatcccagcaatttgggaggccaaggtgggtagatcacctgagattaggagttggagaccagcctggccaatatggtgaaaccccgtctctaccaaaaaaacaaaaattagctgagcctggtcatgcatgcctggaatcccaacaactcgggaggctgaggcaggagaatcgcttgaacccaggaggcggagattgcagtgagccaagattgtgccactgcactcatcgattccaattcagcgggggccacctgatgtcccggccagcagaggaagcaacgcggggaccacgggttaacccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccgtataggaggaccgtgtaggccttcctgtcccgggccttgccaggggccagcccgaagatggagctccctcgcagggggtagcctccgaaggagaagacgtgggagtggtcggcagtgacgaggctcagcgtgtcctcctcgctggtgagctggcccgccctctcaatggcgtcgtcgaacatgatcgtctcagtcagtgcccggtaagccctgctttcatgatgaccatggtcgatgcgaccaccctccacgaagaggaagaagccgcgggggttcctgctcagcaggcgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacataccgggcaccctggcgcttcgccagccattcctgcaccagattcttcccgtccagcctggtcccaccttggctgtagtcatctgggtactcagggtctggggttcccatgcggaacatgtactttcggcctccacctaggatcacgtcaatgtccatgttggagatgagctgcgtagcgatgtcctggcacccctcctggcgggccgaggcaggcacgtcggcgtccgagtaccagttgcggttcaccgtgtgggcgtaggtgccggctggcgaggcgtgctgcactcgtgtggtggttaccactcccactgacttccctgctttcttggcccgattcatcacggagatgacctcgttgccgcgtgtcgtgttgcactggttaaagcgggcggctgcactcaagccaatggtctggaagttgcccttgaccccgcacaggtaggccgtggctgtggctccactgtctggcacatgtttgtctacattgtatgtcttggacagagccacatatgggaagcggtccatggccaggggtatctcaggccccagtttgtccttcttctgcccttttaggatctacgtcgagactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 55 - DNA, 4676 bp, synthetic construct
  misc_feature 1..4676: note = synthetic construct
  source 1..4676: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagtacgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattcgccctgcagcgggaattgccaccatggctcacgttcgaggactgcagctgcctggatgtctggctcttgccgctctgtgtagcctggtgcacagccagcacgtgtttctggctcctcagcaagccagatcactgctgcagcgcgtcagaagaagcgtcagcgagatccagctgatgcacaacctgggcaagcacctgaacagcatggaacgcgtggagtggctgcggaagaaactgcaggacgtgcacaacggtggcggcggaggcggaagcggtggcggaggatcaggcggtggtggatctgacgcccacaagtctgaagtggcccaccggttcaaggacctgggcgaagagaatttcaaggccctggtgctgatcgccttcgctcagtacctgcagcagtgccccttcgaggaccacgtgaagctggtcaacgaagtgaccgagttcgccaagacctgcgtggccgacgagagcgccgagaactgtgataagagcctgcacaccctgttcggcgacaagctgtgtacagtggccacactgagagaaacctacggcgagatggccgactgctgcgccaagcaagagcccgagagaaacgagtgcttcctgcagcacaaggacgacaaccccaacctgcctagactcgtgcggcctgaagtggacgtgatgtgcaccgccttccacgacaacgaggaaaccttcctgaagaagtacctgtacgagatcgccagacggcacccctacttttacgcccctgagctgctgttcttcgccaagcggtataaggccgccttcaccgagtgttgtcaggccgctgataaggctgcctgcctgctgcctaagctggacgagcttagagacgagggcaaagccagctccgccaagcagagactgaagtgtgccagcctgcagaagttcggcgagagagcctttaaggcctgggccgttgctagactgagccagagatttcccaaggccgagtttgccgaggtgtccaagctcgtgaccgacctgacaaaggtgcacaccgagtgctgccacggcgacctgctggaatgcgccgacgatagagccgacctggccaagtacatctgcgagaaccaggacagcatcagcagcaagctgaaagagtgctgcgagaagcctctgctggaaaagagccactgtatcgccgaggtggaaaacgacgagatgcccgccgatctgccttctctggccgccgattttgtggaaagcaaggacgtgtgcaagaactacgccgaggccaaggacgtgttcctgggcatgtttctgtacgagtacgcccgcagacaccccgactactctgttgtgctgctgctgagactggccaaaacctacgagacaaccctggaaaagtgctgtgccgccgctgatcctcacgagtgttacgccaaggtgttcgacgagttcaagccactggtggaagaaccccagaacctgatcaagcagaactgcgagctgttcgagcagctgggcgagtacaagttccagaacgccctgctcgtgcggtacaccaagaaggtgccccaggtttccacacctacactggttgaggtgtcccggaatctgggcaaagtgggcagcaagtgctgcaagcaccctgaggccaagagaatgccttgcgccgaggattacctgagcgtggtgctgaatcagctgtgcgtgctgcacgagaaaacccctgtgtccgacagagtgaccaagtgctgtaccgagagcctggtcaacagacggccttgctttagcgccctcgaggtggacgagacatacgtgcccaaagagttcaacgccgagacattcaccttccacgccgacatctgtaccctgagcgagaaagagcggcagatcaagaaacagactgccctggtggaactggtcaagcacaagcccaaggccaccaaagaacagctgaaggccgtgatggacgacttcgccgccttcgtggaaaaatgctgcaaggccgacgacaaagagacatgcttcgccgaagagggcaagaaactggtggccgcttctcaggctgccctgggatgataaggtaccgatctttttccctctgccaaaaattatggggacatcatgaagccccttgagcatctgacttctggctaataaaggaaatttattttcattgcaatagtgtgttggaattttttgtgtctctcactcggaaggcgcgaccggttgggcatggccaggtagcctatgctgtgtctggacgtcctcctgctggtatagttattttaaaatcagaaggacagggaagggagcagtggttcacgcctgtaatcccagcaatttgggaggccaaggtgggtagatcacctgagattaggagttggagaccagcctggccaatatggtgaaaccccgtctctaccaaaaaaacaaaaattagctgagcctggtcatgcatgcctggaatcccaacaactcgggaggctgaggcaggagaatcgcttgaacccaggaggcggagattgcagtgagccaagattgtgccactgcactcatcgattccaattcagcgggggccacctgatgtcccggccagcagaggaagcaacgcggggaccacgggttaacccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccgtataggaggaccgtgtaggccttcctgtcccgggccttgccaggggccagcccgaagatggagctccctcgcagggggtagcctccgaaggagaagacgtgggagtggtcggcagtgacgaggctcagcgtgtcctcctcgctggtgagctggcccgccctctcaatggcgtcgtcgaacatgatcgtctcagtcagtgcccggtaagccctgctttcatgatgaccatggtcgatgcgaccaccctccacgaagaggaagaagccgcgggggttcctgctcagcaggcgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacataccgggcaccctggcgcttcgccagccattcctgcaccagattcttcccgtccagcctggtcccaccttggctgtagtcatctgggtactcagggtctggggttcccatgcggaacatgtactttcggcctccacctaggatcacgtcaatgtccatgttggagatgagctgcgtagcgatgtcctggcacccctcctggcgggccgaggcaggcacgtcggcgtccgagtaccagttgcggttcaccgtgtgggcgtaggtgccggctggcgaggcgtgctgcactcgtgtggtggttaccactcccactgacttccctgctttcttggcccgattcatcacggagatgacctcgttgccgcgtgtcgtgttgcactggttaaagcgggcggctgcactcaagccaatggtctggaagttgcccttgaccccgcacaggtaggccgtggctgtggctccactgtctggcacatgtttgtctacattgtatgtcttggacagagccacatatgggaagcggtccatggccaggggtatctcaggccccagtttgtccttcttctgcccttttaggatctacgtcgagactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 56 - DNA, 3306 bp, synthetic construct
  misc_feature 1..3306: note = synthetic construct
  source 1..3306: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcaagcttgccaccatgattcccgccaaggacatggccaaagtgatgatcgtgatgctggccatctgctttctgaccaagagcgacggcagcgtgtccgagattcagctgatgcacaacctgggcaagcacctgaacagcatggaacgcgtggagtggctgcggaagaaactgcaggacgtgcacaacttcgtggccctgggagcacctctggctcctagagacgccggcagccagcggcctagaaagaaagaggataacgtcctggtcgagagccacgagaagtctctgggcgaagccgacaaggccgacaagacacacacctgtcctccctgtcctgctcccgaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgacaaagaaccaggtgtccctgacctgcctggtcaagggcttctacccttccgatatcgccgtcgagtgggagtccaacggccagccagagaacaactacaagacaacccctcctgtgctggactccgatggctcattcttcctgtacagcaagctgacagtggacaagagccggtggcagcagggcaatgtgttcagctgtagcgtgatgcacgaggccctgcacaaccactacacccagaagtccctgagcctgtctcctggctgatgaggtacctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 57 - DNA, 3456 bp, synthetic construct
  misc_feature 1..3456: note = synthetic construct
  source 1..3456: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccctgcagcgggaattgccaccatggctcacgttcgaggactgcagctgcctggatgtctggctcttgccgctctgtgtagcctggtgcacagccagcacgtgtttctggctcctcagcaagccagatcactgctgcagcgcgtcagaagaagcgtcagcgagatccagctgatgcacaacctgggcaagcacctgaacagcatggaacgcgtggagtggctgcggaagaaactgcaggacgtgcacaacttcgtggccctgggagcacctctggctccaagagacgccggcagccaacggcctagaaagaaagaggacaacgtcctggtcgagagccacgagaagtctctgggcgaagccgataaggctgacggtggcggaggcggaggatctggtggcggtggttctggcggtggcggatctgccgagtctaagtacggacctccttgtcctccctgtcctgctcccgaagctgctggcggcccatccgtgtttctgttccctccaaagcctaaggacaccctgatgatcagcagaacccctgaagtgacctgcgtggtggtggacgtgtcccaagaggatcctgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtcgagtgggagagcaacggccagcctgagaacaactacaagaccacacctcctgtgctggacagcgacggctcattcttcctgtacagccggctgaccgtggacaagagccggtggcaagagggcaacgtgttcagctgctccgtgatgcacgaggccctgcacaaccactacacccagaaaagcctgtctctgagcctgggctgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 58 - DNA, 3324 bp, synthetic construct
  misc_feature 1..3324: note = synthetic construct
  source 1..3324: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcaagcttgccaccatgattcccgccaaggacatggccaaagtgatgatcgtgatgctggccatctgctttctgaccaagagcgacggcaagagcgtgaagaaaagaagcgtgtccgagatccagctgatgcacaacctgggcaagcacctgaacagcatggaacgcgtggagtggctgcggaagaaactgcaggacgtgcacaacttcgtggccctgggagcacctctggctcctagggacgccggcagccagcggcctagaaagaaagaggataacgtcctggtcgagagccacgagaagtctctgggcgaagccgacaaggccgacaaaacacacacctgtcctccctgtcctgctcccgaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtccacaacgccaagaccaagcctagagaggaacagtacaacagcacctacagagtggtgtccgtgctgaccgtgctgcaccaggattggctgaacggcaaagagtacaagtgcaaggtgtctaacaaggccctgcctgctcctatcgagaaaaccatcagcaaggccaagggccagcctagggaaccccaggtttacacactccctccaagcagggacgagctgacaaagaaccaggtgtccctgacctgcctggtcaagggcttctacccttccgatatcgccgtcgagtgggagtccaacggccagccagagaacaactacaagacaacccctcctgtgctggactccgacggctcattcttcctgtacagcaagctgacagtggacaagagccggtggcagcagggcaatgtgttcagctgtagcgtgatgcacgaggccctgcacaaccactacacccagaagtccctgagcctgtctcctggctgatgaggtacctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 59 - DNA, 4398 bp, synthetic construct; identical to SEQ ID NO: 53
  misc_feature 1..4398: note = synthetic construct
  source 1..4398: mol_type = other DNA

SEQ ID NO: 60 - DNA, 3734 bp, synthetic construct
  misc_feature 1..3734: note = synthetic construct
  source 1..3734: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagtacgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattcgccctgcagcgggaattgccaccatggctcacgttcgaggactgcagctgcctggatgtctggctcttgccgctctgtgtagcctggtgcacagccagcacgtgtttctggctcctcagcaagccagatcactgctgcagcgcgtcagaagaagcgtcagcgagatccagctgatgcacaacctgggcaagcacctgaacagcatggaacgcgtggagtggctgcggaagaaactgcaggacgtgcacaacttcgtggccctgggagcacctctggctccaagagacgccggcagccaacggcctagaaagaaagaggacaacgtcctggtcgagagccacgagaagtctctgggcgaagccgataaggctgacggtggcggaggcggaggatctggtggcggtggttctggcggtggcggatctgccgagtctaagtacggacctccttgtcctccctgtcctgctcccgaagctgctggcggcccatccgtgtttctgttccctccaaagcctaaggacaccctgatgatcagcagaacccctgaagtgacctgcgtggtggtggacgtgtcccaagaggatcctgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtcgagtgggagagcaacggccagcctgagaacaactacaagaccacacctcctgtgctggacagcgacggctcattcttcctgtacagccggctgaccgtggacaagagccggtggcaagagggcaacgtgttcagctgctccgtgatgcacgaggccctgcacaaccactacacccagaaaagcctgtctctgagcctgggctgatgaggtaccgatctttttccctctgccaaaaattatggggacatcatgaagccccttgagcatctgacttctggctaataaaggaaatttattttcattgcaatagtgtgttggaattttttgtgtctctcactcggaaggcgcgaccggttgggcatggccaggtagcctatgctgtgtctggacgtcctcctgctggtatagttattttaaaatcagaaggacagggaagggagcagtggttcacgcctgtaatcccagcaatttgggaggccaaggtgggtagatcacctgagattaggagttggagaccagcctggccaatatggtgaaaccccgtctctaccaaaaaaacaaaaattagctgagcctggtcatgcatgcctggaatcccaacaactcgggaggctgaggcaggagaatcgcttgaacccaggaggcggagattgcagtgagccaagattgtgccactgcactcatcgattccaattcagcgggggccacctgatgtcccggccagcagaggaagcaacgcggggaccacgggttaacccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccgtataggaggaccgtgtaggccttcctgtcccgggccttgccaggggccagcccgaagatggagctccctcgcagggggtagcctccgaaggagaagacgtgggagtggtcggcagtgacgaggctcagcgtgtcctcctcgctggtgagctggcccgccctctcaatggcgtcgtcgaacatgatcgtctcagtcagtgcccggtaagccctgctttcatgatgaccatggtcgatgcgaccaccctccacgaagaggaagaagccgcgggggttcctgctcagcaggcgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacataccgggcaccctggcgcttcgccagccattcctgcaccagattcttcccgtccagcctggtcccaccttggctgtagtcatctgggtactcagggtctggggttcccatgcggaacatgtactttcggcctccacctaggatcacgtcaatgtccatgttggagatgagctgcgtagcgatgtcctggcacccctcctggcgggccgaggcaggcacgtcggcgtccgagtaccagttgcggttcaccgtgtgggcgtaggtgccggctggcgaggcgtgctgcactcgtgtggtggttaccactcccactgacttccctgctttcttggcccgattcatcacggagatgacctcgttgccgcgtgtcgtgttgcactggttaaagcgggcggctgcactcaagccaatggtctggaagttgcccttgaccccgcacaggtaggccgtggctgtggctccactgtctggcacatgtttgtctacattgtatgtcttggacagagccacatatgggaagcggtccatggccaggggtatctcaggccccagtttgtccttcttctgcccttttaggatctacgtcgagactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 61 - DNA, 3624 bp, synthetic construct
  misc_feature 1..3624: note = synthetic construct
  source 1..3624: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagtacgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattcaagcttgccaccatgattcccgccaaggacatggccaaagtgatgatcgtgatgctggccatctgctttctgaccaagagcgacggcaagagcgtgaagaaaagaagcgtgtccgagatccagctgatgcacaacctgggcaagcacctgaacagcatggaacgcgtggagtggctgcggaagaaactgcaggacgtgcacaacttcgtggccctgggagcacctctggctcctagggacgccggcagccagcggcctagaaagaaagaggataacgtcctggtcgagagccacgagaagtctctgggcgaagccgacaaggccgacaaaacacacacctgtcctccctgtcctgctcccgaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtccacaacgccaagaccaagcctagagaggaacagtacaacagcacctacagagtggtgtccgtgctgaccgtgctgcaccaggattggctgaacggcaaagagtacaagtgcaaggtgtctaacaaggccctgcctgctcctatcgagaaaaccatcagcaaggccaagggccagcctagggaaccccaggtttacacactccctccaagcagggacgagctgacaaagaaccaggtgtccctgacctgcctggtcaagggcttctacccttccgatatcgccgtcgagtgggagtccaacggccagccagagaacaactacaagacaacccctcctgtgctggactccgacggctcattcttcctgtacagcaagctgacagtggacaagagccggtggcagcagggcaatgtgttcagctgtagcgtgatgcacgaggccctgcacaaccactacacccagaagtccctgagcctgtctcctggctgatgaggtaccgatctttttccctctgccaaaaattatggggacatcatgaagccccttgagcatctgacttctggctaataaaggaaatttattttcattgcaatagtgtgttggaattttttgtgtctctcactcggaaggcgcgaccggttgggcatggccaggtagcctatgctgtgtctggacgtcctcctgctggtatagttattttaaaatcagaaggacagggaagggagcagtggttcacgcctgtaatcccagcaatttgggaggccaaggtgggtagatcacctgagattaggagttggagaccagcctggccaatatggtgaaaccccgtctctaccaaaaaaacaaaaattagctgagcctggtcatgcatgcctggaatcccaacaactcgggaggctgaggcaggagaatcgcttgaacccaggaggcggagattgcagtgagccaagattgtgccactgcactcatcgattccaattcagcgggggccacctgatgtcccggccagcagaggaagcaacgcggggaccacgggttaacccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccgtataggaggaccgtgtaggccttcctgtcccgggccttgccaggggccagcccgaagatggagctccctcgcagggggtagcctccgaaggagaagacgtgggagtggtcggcagtgacgaggctcagcgtgtcctcctcgctggtgagctggcccgccctctcaatggcgtcgtcgaacatgatcgtctcagtcagtgcccggtaagccctgctttcatgatgaccatggtcgatgcgaccaccctccacgaagaggaagaagccgcgggggttcctgctcagcaggcgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacataccgggcaccctggcgcttcgccagccattcctgcaccagattcttcccgtccagcctggtcccaccttggctgtagtcatctgggtactcagggtctggggttcccatgcggaacatgtactttcggcctccacctaggatcacgtcaatgtccatgttggagatgagctgcgtagcgatgtcctggcacccctcctggcgggccgaggcaggcacgtcggcgtccgagtaccagttgcggttcaccgtgtgggcgtaggtgccggctggcgaggcgtgctgcactcgtgtggtggttaccactcccactgacttccctgctttcttggcccgattcatcacggagatgacctcgttgccgcgtgtcgtgttgcactggttaaagcgggcggctgcactcaagccaatggtctggaagttgcccttgaccccgcacaggtaggccgtggctgtggctccactgtctggcacatgtttgtctacattgtatgtcttggacagagccacatatgggaagcggtccatggccaggggtatctcaggccccagtttgtccttcttctgcccttttaggatctacgtcgagactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcca

SEQ ID NO: 62 - DNA, 4799 bp, synthetic construct
  misc_feature 1..4799: note = synthetic construct
  source 1..4799: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagtacgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattcgccctgcagcgggaattgccaccatggctcacgttcgaggactgcagctgcctggatgtctggctcttgccgctctgtgtagcctggtgcacagccagcacgtgtttctggctcctcagcaagccagatcactgctgcagcgcgtcagaagaagcgtcagcgagatccagctgatgcacaacctgggcaagcacctgaacagcatggaacgcgtggagtggctgcggaagaaactgcaggacgtgcacaacttcgtggccctgggagcacctctggctccaagagacgccggcagccaacggcctagaaagaaagaggacaacgtcctggtcgagagccacgagaagtctctgggcgaagccgataaggctgacggtggcggaggcggaggatctggtggcggtggttctggcggtggcggatctgacgcccacaaatctgaagtggcccaccggttcaaggacctgggagaagagaacttcaaggccctggtgctgatcgccttcgctcagtacctgcagcagtgccccttcgaggaccacgtgaagctggtcaacgaagtgaccgagttcgccaagacctgcgtggccgacgagagcgccgagaactgtgataagagcctgcacaccctgttcggcgacaagctgtgtacagtggccacactgagagaaacctacggcgagatggccgactgctgcgccaagcaagagcccgagagaaacgagtgcttcctgcagcacaaggacgacaaccccaacctgcctagactcgtgcggcctgaagtggacgtgatgtgcaccgccttccacgacaacgaggaaaccttcctgaagaagtacctgtacgagatcgccagacggcacccctacttttacgcccctgagctgctgttcttcgccaagcggtataaggccgccttcaccgagtgttgtcaggccgctgataaggcagcctgcctgctgcctaagctggacgagcttagagacgagggcaaagccagctccgccaagcagagactgaagtgtgccagcctgcagaagttcggcgagagagcctttaaggcctgggccgttgctagactgagccagagatttcccaaggccgagtttgccgaggtgtccaagctcgtgaccgacctgacaaaggtgcacaccgagtgctgccacggcgacctgctggaatgcgccgacgatagagccgacctggccaagtacatctgcgagaaccaggacagcatcagcagcaagctgaaagagtgctgcgagaagcctctgctggaaaagagccactgtatcgccgaggtggaaaacgacgagatgcccgccgatctgccttctctggccgccgattttgtggaaagcaaggacgtgtgcaagaactacgccgaggccaaggacgtgttcctgggcatgtttctgtacgagtacgcccgcagacaccccgactactctgttgtgctgctgctgagactggccaaaacctacgagacaaccctggaaaagtgctgtgccgccgctgatcctcacgagtgttacgccaaggtgttcgacgagttcaagccactggtggaagaaccccagaacctgatcaagcagaactgcgagctgttcgagcagctgggcgagtacaagttccagaacgccctgctcgtgcggtacaccaagaaggtgccccaggtttccacacctacactggttgaggtgtcccggaatctgggcaaagtgggcagcaagtgctgcaagcaccctgaggccaagagaatgccttgcgccgaggattacctgagcgtggtgctgaatcagctgtgcgtgctgcacgagaaaacccctgtgtccgacagagtgaccaagtgctgtaccgagagcctggtcaacagacggccttgctttagcgccctcgaggtggacgagacatacgtgcccaaagagttcaacgccgagacattcaccttccacgccgacatctgtaccctgagcgagaaagagcggcagatcaagaaacagactgccctggtggaactggtcaagcacaagcccaaggccaccaaagaacagctgaaggccgtgatggacgacttcgccgccttcgtggaaaaatgctgcaaggccgacgacaaagagacatgcttcgccgaagagggcaagaaactggtggccgcttctcaggctgccctgggatgataaggtaccgatctttttccctctgccaaaaattatggggacatcatgaagccccttgagcatctgacttctggctaataaaggaaatttattttcattgcaatagtgtgttggaattttttgtgtctctcactcggaaggcgcgaccggttgggcatggccaggtagcctatgctgtgtctggacgtcctcctgctggtatagttattttaaaatcagaaggacagggaagggagcagtggttcacgcctgtaatcccagcaatttgggaggccaaggtgggtagatcacctgagattaggagttggagaccagcctggccaatatggtgaaaccccgtctctaccaaaaaaacaaaaattagctgagcctggtcatgcatgcctggaatcccaacaactcgggaggctgaggcaggagaatcgcttgaacccaggaggcggagattgcagtgagccaagattgtgccactgcactcatcgattccaattcagcgggggccacctgatgtcccggccagcagaggaagcaacgcggggaccacgggttaacccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccgtataggaggaccgtgtaggccttcctgtcccgggccttgccaggggccagcccgaagatggagctccctcgcagggggtagcctccgaaggagaagacgtgggagtggtcggcagtgacgaggctcagcgtgtcctcctcgctggtgagctggcccgccctctcaatggcgtcgtcgaacatgatcgtctcagtcagtgcccggtaagccctgctttcatgatgaccatggtcgatgcgaccaccctccacgaagaggaagaagccgcgggggttcctgctcagcaggcgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacataccgggcaccctggcgcttcgccagccattcctgcaccagattcttcccgtccagcctggtcccaccttggctgtagtcatctgggtactcagggtctggggttcccatgcggaacatgtactttcggcctccacctaggatcacgtcaatgtccatgttggagatgagctgcgtagcgatgtcctggcacccctcctggcgggccgaggcaggcacgtcggcgtccgagtaccagttgcggttcaccgtgtgggcgtaggtgccggctggcgaggcgtgctgcactcgtgtggtggttaccactcccactgacttccctgctttcttggcccgattcatcacggagatgacctcgttgccgcgtgtcgtgttgcactggttaaagcgggcggctgcactcaagccaatggtctggaagttgcccttgaccccgcacaggtaggccgtggctgtggctccactgtctggcacatgtttgtctacattgtatgtcttggacagagccacatatgggaagcggtccatggccaggggtatctcaggccccagtttgtccttcttctgcccttttaggatctacgtcgagactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 63 - DNA, 3309 bp, synthetic construct; identical to SEQ ID NO: 45
  misc_feature 1..3309: note = synthetic construct
  source 1..3309: mol_type = other DNA

SEQ ID NO: 64 - DNA, 3327 bp, synthetic construct
  misc_feature 1..3327: note = synthetic construct
  source 1..3327: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcaagcttgccaccatgattcccgccaaggacatggccaaagtgatgatcgtgatgctggccatctgctttctgaccaagagcgacggcaagagcgtgaagaaaagaagcgtgtccgagatccagctgatgcacaacctgggcaagcacctgaacagcatggaacgcgtggagtggctgcggaagaaactgcaggacgtgcacaacttcgtggccctgggagcacctctggctcctagagacgccggcagccagcggcctagaaagaaagaggataacgtcctggtcgagagccacgagaagtctctgggcgaagccgacaaggccgacgacaagacacacacctgtcctccctgtcctgctcccgaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgacaaagaaccaggtgtccctgacctgcctggtcaagggcttctacccttccgatatcgccgtcgagtgggagtccaacggccagccagagaacaactacaagacaacccctcctgtgctggactccgacggctcattcttcctgtacagcaagctgacagtggacaagagccggtggcagcagggcaatgtgttcagctgtagcgtgatgcacgaggccctgcacaaccactacacccagaagtccctgagcctgtctcctggctgatgaggtacctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 65 - DNA, 3312 bp, synthetic construct; identical to SEQ ID NO: 43
  misc_feature 1..3312: note = synthetic construct
  source 1..3312: mol_type = other DNA

SEQ ID NO: 66 - DNA, 3330 bp, synthetic construct
  misc_feature 1..3330: note = synthetic construct
  source 1..3330: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcaagcttgccaccatgattcccgccaaggacatggccaaagtgatgatcgtgatgctggccatctgctttctgaccaagagcgacggcaagagcgtgaagaaaagaagcgtgtccgagatccagctgatgcacaacctgggcaagcacctgaacagcatggaacgcgtggagtggctgcggaagaaactgcaggacgtgcacaacttcgtggccctgggagcacctctggctcctagagacgccggcagccagcggcctagaaagaaagaggataacgtcctggtcgagagccacgagaagtctctgggcgaagccgacaaggccgacgtggacaagacacacacctgtcctccctgtcctgctcccgaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgacaaagaaccaggtgtccctgacctgcctggtcaagggcttctacccttccgatatcgccgtcgagtgggagtccaacggccagccagagaacaactacaagacaacccctcctgtgctggactccgacggctcattcttcctgtacagcaagctgacagtggacaagagccggtggcagcagggcaatgtgttcagctgtagcgtgatgcacgaggccctgcacaaccactacacccagaagtccctgagcctgtctcctggctgatgaggtacctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 67 - DNA, 2700 bp, synthetic construct
  misc_feature 1..2700: note = synthetic construct
  source 1..2700: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccttgccctgcagcgggaattgccaccatgtacaagatgcagctgctgagctgtatcgccctgacactggtgctggtggccaactccaagagcgtgaagaaaagaagcgtgtccgagatccagctgatgcacaacctgggcaagcacctgaacagcatggaacgcgtggagtggctgcggaagaaactgcaggacgtgcacaacttcgtggccctgggagcacctctggctcctagagacgccggcagccagcggcctagaaagaaagaggataacgtcctggtcgagagccacgagaagtctctgggcgaagccgataaggccgacgtgaacgtgctgaccaaggccaagagccagtgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 68 - DNA, 2715 bp, synthetic construct
  misc_feature 1..2715: note = synthetic construct
  source 1..2715: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccttgccctgcagcgggaattgccaccatgattcccgccaaggacatggccaaagtgatgatcgtgatgctggccatctgctttctgaccaagagcgacggcaagagcgtgaagaaaagaagcgtgtccgagatccagctgatgcacaacctgggcaagcacctgaacagcatggaacgcgtggagtggctgcggaagaaactgcaggacgtgcacaacttcgtggccctgggagcacctctggctcctagagacgccggcagccagcggcctagaaagaaagaggataacgtcctggtcgagagccacgagaagtctctgggcgaagccgataaggccgacgtgaacgtgctgaccaaggccaagagccagtgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 69 - DNA, 3486 bp, synthetic construct
  misc_feature 1..3486: note = synthetic construct
  source 1..3486: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccctgcagcgggaattgccaccatggctcacgttcgaggactgcagctgcctggatgtctggctcttgccgctctgtgtagcctggtgcacagccagcacgtgtttctggctcctcagcaagccagatcactgctgcagcgcgtcagaagaagcgtcagcgagatccagctgatgcacaacctgggcaagcacctgaacagcatggaacgcgtggagtggctgcggaagaaactgcaggacgtgcacaacttcgtggccctgggagcacctctggctccaagagacgccggcagccaacggcctagaaagaaagaggacaacgtcctggtcgagagccacgagaagtctctgggcgaagccgataaggccgacgtgaacgtgctgaccaaggccaagtctcaaggtggcggaggcggaggatctggtggtggtggatctggcggcggaggttctgccgagtctaagtacggacctccttgtcctccctgtcctgctcccgaagctgctggcggcccatccgtgtttctgttccctccaaagcctaaggacaccctgatgatcagcagaacccctgaagtgacctgcgtggtggtcgacgtgtcccaagaggatcctgaggtgcagttcaattggtacgtggacggcgtggaagtgcacaacgccaagaccaagcctagagaggaacagttcaacagcacctacagagtggtgtctgtgctgaccgtgctgcaccaggattggctgaacggcaaagagtacaagtgcaaggtgtccaacaagggcctgcctagcagcatcgagaaaaccatctctaaggccaagggccagcctcgcgaaccccaggtttacacactgcctccaagccaagaggaaatgaccaagaaccaggtgtccctgacctgcctggtcaagggcttctacccttccgatatcgccgtcgagtgggagagcaacggccagcctgagaacaactacaagaccacacctcctgtgctggacagcgacggctcattcttcctgtacagccggctgaccgtggacaagagcaggtggcaagagggcaacgtgttcagctgctccgtgatgcacgaggccctgcacaaccactacacccagaaaagcctgtctctgagcctgggctgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 70 - DNA, 4551 bp, synthetic construct
  misc_feature 1..4551: note = synthetic construct
  source 1..4551: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccctgcagcgggaattgccaccatggctcacgttcgaggactgcagctgcctggatgtctggctcttgccgctctgtgtagcctggtgcacagccagcacgtgtttctggctcctcagcaagccagatcactgctgcagcgcgtcagaagaagcgtcagcgagatccagctgatgcacaacctgggcaagcacctgaacagcatggaacgcgtggagtggctgcggaagaaactgcaggacgtgcacaacttcgtggccctgggagcacctctggctccaagagacgccggcagccaacggcctagaaagaaagaggacaacgtcctggtcgagagccacgagaagtctctgggcgaagccgataaggccgacgtgaacgtgctgaccaaggccaagtctcaaggtggcggaggcggaggatctggtggtggtggatctggcggcggaggttctgacgcccacaaatctgaagtggcccaccggttcaaggacctgggagaagagaacttcaaggccctggtgctgatcgccttcgctcagtacctgcagcagtgccccttcgaggaccacgtgaagctggtcaacgaagtgaccgagttcgccaagacctgcgtggccgacgagagcgccgagaactgtgataagagcctgcacaccctgttcggcgacaagctgtgtacagtggccacactgagagaaacctacggcgagatggccgactgctgcgccaagcaagagcccgagagaaacgagtgcttcctgcagcacaaggacgacaaccccaacctgcctagactcgtgcggcctgaagtggacgtgatgtgcaccgccttccacgacaacgaggaaaccttcctgaagaagtacctgtacgagatcgccagacggcacccctacttttacgcccctgagctgctgttcttcgccaagcggtataaggccgccttcaccgagtgttgtcaggccgctgataaggctgcctgcctgctgcctaagctggacgagcttagagacgagggcaaagccagctccgccaagcagagactgaagtgtgccagcctgcagaagttcggcgagagagcctttaaggcctgggccgttgctagactgagccagagatttcccaaggccgagtttgccgaggtgtccaagctcgtgaccgacctgacaaaggtgcacaccgagtgctgccacggcgacctgctggaatgcgccgacgatagagccgacctggccaagtacatctgcgagaaccaggacagcatcagcagcaagctgaaagagtgctgcgagaagcctctgctggaaaagagccactgtatcgccgaggtggaaaacgacgagatgcccgccgatctgccttctctggccgccgattttgtggaaagcaaggacgtgtgcaagaactacgccgaggccaaggacgtgttcctgggcatgtttctgtacgagtacgcccgcagacaccccgactactctgttgtgctgctgctgagactggccaaaacctacgagacaaccctggaaaagtgctgtgccgccgctgatcctcacgagtgttacgccaaggtgttcgacgagttcaagccactggtggaagaaccccagaacctgatcaagcagaactgcgagctgttcgagcagctgggcgagtacaagttccagaacgccctgctcgtgcggtacaccaagaaggtgccccaggtttccacacctacactggttgaggtgtcccggaatctgggcaaagtgggcagcaagtgctgcaagcaccctgaggccaagagaatgccttgcgccgaggattacctgagcgtggtgctgaatcagctgtgcgtgctgcacgagaaaacccctgtgtccgacagagtgaccaagtgctgtaccgagagcctggtcaacagacggccttgctttagcgccctcgaggtggacgagacatacgtgcccaaagagttcaacgccgagacattcaccttccacgccgacatctgtaccctgagcgagaaagagcggcagatcaagaaacagactgccctggtggaactggtcaagcacaagcccaaggccaccaaagaacagctgaaggccgtgatggacgacttcgccgccttcgtggaaaaatgctgcaaggccgacgacaaagagacatgcttcgccgaagagggcaagaaactggtggccgcttctcaggctgccctgggatgataaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 71 - DNA, 4744 bp, synthetic construct
  misc_feature 1..4744: note = synthetic construct
  source 1..4744: mol_type = other DNA
ctgcgcgctcgctcgctcactgaggccgcccgggcaaagcccgggcggcctcagtgagcgagcgagcgcgcagagagggagtggccaactccatcactaggggttccttgtagttaatgattaacccgccatgctacttatctacgtagccatgctctagtacgcgttcgagctcgccccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgggggatcttggtggcgtgaaactcccgcagatcttcggccagcgaattccagaagccaccatggactatcctgctgccaagagggtcaagttggactctagaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcctccaagcctgggacctctattatcatgtgttccgacgaatctcaaagactagagatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctagtaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggactatcctgctgccaagagggtcaagttggactctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagaggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagaggcgttcaggtggaaaccatcagtccaggggatggccgaacttttccaaagagagggcagacttgcgtcgtgcattatactggtatgctggaggatgggaaaaagttcgactcttccagagatcggaacaaaccattcaaattcatgctcgggaaacaggaagttatccgcggatgggaggagggcgtggcccagatgtccgtgggccagcgcgccaagctaaccatctccccagactacgcctacggagccaccggacaccccggtatcatacccccacacgccacccttgtgtttgacgtggaactgcttaagctagagactagaggcgtgcaggtcgagaccatcagccccggcgacggccgcacctttcccaagagaggccagacttgcgtggtccactacaccggcatgctggaggacggcaagaagttcgacagcagccgcgaccgcaacaagcccttcaagttcatgctgggcaaacaggaagtgatccgcggctgggaggaaggcgtggctcagatgagcgtggggcagcgggccaagctgaccatcagccccgactatgcctacggcgccaccggccaccccggcatcatccccccccacgccaccctggtgttcgacgtggagctgctgaagctggagtgataaggatccccactccagtgcccaccagccttgtcctaataaaattaagttgcatcattttgtctgactaggtgtccttctataatattatggggtggaggggggtggtttggagcaaggcgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattgccaccatgtacaagatgcagctgctgagctgtatcgccctgacactggtgctggtggccaactccaagagcgtgaagaaaagaagcgtgtccgagatccagctgatgcacaacctgggcaagcacctgaacagcatggaacgcgtggagtggctgcggaagaaactgcaggacgtgcacaacttcgtggccctgggagcacctctggctcctagagacgccggcagccagcggcctagaaagaaagaggataacgtcctggtcgagagccacgagaagtctctgggcgaagccgataaggccgacgtgaacgtgctgaccaaggccaagagccagtgatgaggtaccgatctttttccctctgccaaaaattatggggacatcatgaagccccttgagcatctgacttctggctaataaaggaaatttattttcattgcaatagtgtgttggaattttttgtgtctctcactcggaaggcgcgccgatcctcgagactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgca

SEQ ID NO: 72 - DNA, 4782 bp, synthetic construct
  misc_feature 1..4782: note = synthetic construct
  source 1..4782: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagtacgcgttcgagctcgccccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgggggatcttggtggcgtgaaactcccgcagatcttcggccagcgaattccagaagccaccatggactatcctgctgccaagagggtcaagttggactctagaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcctccaagcctgggacctctattatcatgtgttccgacgaatctcaaagactagagatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctagtaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggactatcctgctgccaagagggtcaagttggactctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagaggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagaggcgttcaggtggaaaccatcagtccaggggatggccgaacttttccaaagagagggcagacttgcgtcgtgcattatactggtatgctggaggatgggaaaaagttcgactcttccagagatcggaacaaaccattcaaattcatgctcgggaaacaggaagttatccgcggatgggaggagggcgtggcccagatgtccgtgggccagcgcgccaagctaaccatctccccagactacgcctacggagccaccggacaccccggtatcatacccccacacgccacccttgtgtttgacgtggaactgcttaagctagagactagaggcgtgcaggtcgagaccatcagccccggcgacggccgcacctttcccaagagaggccagacttgcgtggtccactacaccggcatgctggaggacggcaagaagttcgacagcagccgcgaccgcaacaagcccttcaagttcatgctgggcaaacaggaagtgatccgcggctgggaggaaggcgtggctcagatgagcgtggggcagcgggccaagctgaccatcagccccgactatgcctacggcgccaccggccaccccggcatcatccccccccacgccaccctggtgttcgacgtggagctgctgaagctggagtgataaggatccccactccagtgcccaccagccttgtcctaataaaattaagttgcatcattttgtctgactaggtgtccttctataatattatggggtggaggggggtggtttggagcaaggcgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattgccaccatgattcccgccaaggacatggccaaagtgatgatcgtgatgctggccatctgctttctgaccaagagcgacggcaagagcgtgaagaaaagaagcgtgtccgagatccagctgatgcacaacctgggcaagcacctgaacagcatggaacgcgtggagtggctgcggaagaaactgcaggacgtgcacaacttcgtggccctgggagcacctctggctcctagagacgccggcagccagcggcctagaaagaaagaggataacgtcctggtcgagagccacgagaagtctctgggcgaagccgataaggccgacgtgaacgtgctgaccaaggccaagagccagtgatgaggtaccgatctttttccctctgccaaaaattatggggacatcatgaagccccttgagcatctgacttctggctaataaaggaaatttattttcattgcaatagtgtgttggaattttttgtgtctctcactcggaaggcgcgccgatcctcgagactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 73 - DNA, 3003 bp, synthetic construct
  misc_feature 1..3003: note = synthetic construct
  source 1..3003: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagtacgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattcgcccttttccttgccctgcagcgggaattgccaccatgattcccgccaaggacatggccaaagtgatgatcgtgatgctggccatctgctttctgaccaagagcgacggcaagagcgtgaagaaaagaagcgtgtccgagatccagctgatgcacaacctgggcaagcacctgaacagcatggaacgcgtggagtggctgcggaagaaactgcaggacgtgcacaacttcgtggccctgggagcacctctggctcctagagacgccggcagccagcggcctagaaagaaagaggataacgtcctggtcgagagccacgagaagtctctgggcgaagccgataaggccgacgtgaacgtgctgaccaaggccaagagccagtgatgaggtaccgatctttttccctctgccaaaaattatggggacatcatgaagccccttgagcatctgacttctggctaataaaggaaatttattttcattgcaatagtgtgttggaattttttgtgtctctcactcggaaggcgcgaccggttgggcatggccaggtagcctatgctgtgtctggacgtcctcctgctggtatagttattttaaaatcagaaggacagggaagggagcagtggttcacgcctgtaatcccagcaatttgggaggccaaggtgggtagatcacctgagattaggagttggagaccagcctggccaatatggtgaaaccccgtctctaccaaaaaaacaaaaattagctgagcctggtcatgcatgcctggaatcccaacaactcgggaggctgaggcaggagaatcgcttgaacccaggaggcggagattgcagtgagccaagattgtgccactgcactcatcgattccaattcagcgggggccacctgatgtcccggccagcagaggaagcaacgcggggaccacgggttaacccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccgtataggaggaccgtgtaggccttcctgtcccgggccttgccaggggccagcccgaagatggagctccctcgcagggggtagcctccgaaggagaagacgtgggagtggtcggcagtgacgaggctcagcgtgtcctcctcgctggtgagctggcccgccctctcaatggcgtcgtcgaacatgatcgtctcagtcagtgcccggtaagccctgctttcatgatgaccatggtcgatgcgaccaccctccacgaagaggaagaagccgcgggggttcctgctcagcaggcgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacataccgggcaccctggcgcttcgccagccattcctgcaccagattcttcccgtccagcctggtcccaccttggctgtagtcatctgggtactcagggtctggggttcccatgcggaacatgtactttcggcctccacctaggatcacgtcaatgtccatgttggagatgagctgcgtagcgatgtcctggcacccctcctggcgggccgaggcaggcacgtcggcgtccgagtaccagttgcggttcaccgtgtgggcgtaggtgccggctggcgaggcgtgctgcactcgtgtggtggttaccactcccactgacttccctgctttcttggcccgattcatcacggagatgacctcgttgccgcgtgtcgtgttgcactggttaaagcgggcggctgcactcaagccaatggtctggaagttgcccttgaccccgcacaggtaggccgtggctgtggctccactgtctggcacatgtttgtctacattgtatgtcttggacagagccacatatgggaagcggtccatggccaggggtatctcaggccccagtttgtccttcttctgcccttttaggatctacgtcgagactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagc

SEQ ID NO: 74 - DNA, 2774 bp, synthetic construct
  misc_feature 1..2774: note = synthetic construct
  source 1..2774: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcaagcttgccaccatgattcccgccaaggacatggccaaagtgatgatcgtgatgctggccatctgctttctgaccaagagcgacggcagcgtgtccgagattcagctgatgcacaacctgggcaagcacctgaacagcatggaacgcgtggagtggctgcggaagaaactgcaggacgtgcacaacttcgacaagacccacacctgtcctccctgtcctgctcccgaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgacaaagaaccaggtgtccctgacctgcctggtcaagggcttctacccttccgatatcgccgtcgagtgggagtccaacggccagccagagaacaactacaagacaacccctcctgtgctggactccgatggctcattcttcctgtacagcaagctgacagtggacaagagccggtggcagcagggcaatgtgttcagctgtagcgtgatgcacgaggccctgcacaaccactacacccagaagtccctgagcctgtctcctggctgatgaggtacctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 75 - DNA, 2918 bp, synthetic construct
  misc_feature 1..2918: note = synthetic construct
  source 1..2918: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccctgcagcgggaattgccaccatggctcacgttcgaggactgcagctgcctggatgtctggctcttgccgctctgtgtagcctggtgcacagccagcacgtgtttctggctcctcagcaagccagatcactgctgcagcgcgtcagaagaagcgtcagcgagatccagctgatgcacaacctgggcaagcacctgaacagcatggaacgcgtggagtggctgcggaagaaactgcaggacgtgcacaacggtggcggcggaggcggaagcggtggcggaggatcaggcggtggtggatctgccgagtctaagtacggacctccttgtcctccctgtcctgctcccgaagctgctggcggcccatccgtgtttctgttccctccaaagcctaaggacaccctgatgatcagcagaacccctgaagtgacctgcgtggtggtggacgtgtcccaagaggatcctgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtcgagtgggagagcaacggccagcctgagaacaactacaagaccacacctcctgtgctggacagcgacggctcattcttcctgtacagccggctgaccgtggacaagagcaggtggcaagagggcaacgtgttcagctgctccgtgatgcacgaggccctgcacaaccactacacccagaagtctctgagcctgagcctgggctgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 76 - DNA, 2792 bp, synthetic construct
  misc_feature 1..2792: note = synthetic construct
  source 1..2792: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcaagcttgccaccatgattcccgccaaggacatggccaaagtgatgatcgtgatgctggccatctgctttctgaccaagagcgacggcaagagcgtgaagaaaagaagcgtgtccgagatccagctgatgcacaacctgggcaagcacctgaacagcatggaacgcgtggagtggctgcggaagaaactgcaggacgtgcacaacttcgacaagacccacacctgtcctccctgtcctgctcccgaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgacaaagaaccaggtgtccctgacctgcctggtcaagggcttttaccccagcgacattgccgtcgagtgggagtctaacggccagcctgagaacaactacaagacaacccctcctgtgctggactccgacggctcattcttcctgtacagcaagctgacagtggacaagagccggtggcagcagggcaatgtgttcagctgtagcgtgatgcacgaggccctgcacaaccactacacccagaagtccctgagcctgtctcctggctgatgaggtacctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 77 - DNA, 3983 bp, synthetic construct
  misc_feature 1..3983: note = synthetic construct
  source 1..3983: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccctgcagcgggaattgccaccatggctcacgttcgaggactgcagctgcctggatgtctggctcttgccgctctgtgtagcctggtgcacagccagcacgtgtttctggctcctcagcaagccagatcactgctgcagcgcgtcagaagaagcgtcagcgagatccagctgatgcacaacctgggcaagcacctgaacagcatggaacgcgtggagtggctgcggaagaaactgcaggacgtgcacaacggtggcggcggaggcggaagcggtggcggaggatcaggcggtggtggatctgacgcccacaagtctgaagtggcccaccggttcaaggacctgggcgaagagaatttcaaggccctggtgctgatcgccttcgctcagtacctgcagcagtgccccttcgaggaccacgtgaagctggtcaacgaagtgaccgagttcgccaagacctgcgtggccgacgagagcgccgagaactgtgataagagcctgcacaccctgttcggcgacaagctgtgtacagtggccacactgagagaaacctacggcgagatggccgactgctgcgccaagcaagagcccgagagaaacgagtgcttcctgcagcacaaggacgacaaccccaacctgcctagactcgtgcggcctgaagtggacgtgatgtgcaccgccttccacgacaacgaggaaaccttcctgaagaagtacctgtacgagatcgccagacggcacccctacttttacgcccctgagctgctgttcttcgccaagcggtataaggccgccttcaccgagtgttgtcaggccgctgataaggctgcctgcctgctgcctaagctggacgagcttagagacgagggcaaagccagctccgccaagcagagactgaagtgtgccagcctgcagaagttcggcgagagagcctttaaggcctgggccgttgctagactgagccagagatttcccaaggccgagtttgccgaggtgtccaagctcgtgaccgacctgacaaaggtgcacaccgagtgctgccacggcgacctgctggaatgcgccgacgatagagccgacctggccaagtacatctgcgagaaccaggacagcatcagcagcaagctgaaagagtgctgcgagaagcctctgctggaaaagagccactgtatcgccgaggtggaaaacgacgagatgcccgccgatctgccttctctggccgccgattttgtggaaagcaaggacgtgtgcaagaactacgccgaggccaaggacgtgttcctgggcatgtttctgtacgagtacgcccgcagacaccccgactactctgttgtgctgctgctgagactggccaaaacctacgagacaaccctggaaaagtgctgtgccgccgctgatcctcacgagtgttacgccaaggtgttcgacgagttcaagccactggtggaagaaccccagaacctgatcaagcagaactgcgagctgttcgagcagctgggcgagtacaagttccagaacgccctgctcgtgcggtacaccaagaaggtgccccaggtttccacacctacactggttgaggtgtcccggaatctgggcaaagtgggcagcaagtgctgcaagcaccctgaggccaagagaatgccttgcgccgaggattacctgagcgtggtgctgaatcagctgtgcgtgctgcacgagaaaacccctgtgtccgacagagtgaccaagtgctgtaccgagagcctggtcaacagacggccttgctttagcgccctcgaggtggacgagacatacgtgcccaaagagttcaacgccgagacattcaccttccacgccgacatctgtaccctgagcgagaaagagcggcagatcaagaaacagactgccctggtggaactggtcaagcacaagcccaaggccaccaaagaacagctgaaggccgtgatggacgacttcgccgccttcgtggaaaaatgctgcaaggccgacgacaaagagacatgcttcgccgaagagggcaagaaactggtggccgcttctcaggctgccctgggatgataaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 78 - DNA, 3611 bp, synthetic construct; identical to SEQ ID NO: 54
  misc_feature 1..3611: note = synthetic construct
  source 1..3611: mol_type = other DNA

SEQ ID NO: 79 - DNA, 2578 bp, synthetic construct
  misc_feature 1..2578: note = synthetic construct
  source 1..2578: mol_type = other DNA
acgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattcgccctgcagcgggaattgccaccatggctcacgttcgaggactgcagctgcctggatgtctggctcttgccgctctgtgtagcctggtgcacagccagcacgtgtttctggctcctcagcaagccagatcactgctgcagcgcgtcagaagaagcgtcagcgagatccagctgatgcacaacctgggcaagcacctgaacagcatggaacgcgtggagtggctgcggaagaaactgcaggacgtgcacaacggtggcggcggaggcggaagcggtggcggaggatcaggcggtggtggatctgacgcccacaagtctgaagtggcccaccggttcaaggacctgggcgaagagaatttcaaggccctggtgctgatcgccttcgctcagtacctgcagcagtgccccttcgaggaccacgtgaagctggtcaacgaagtgaccgagttcgccaagacctgcgtggccgacgagagcgccgagaactgtgataagagcctgcacaccctgttcggcgacaagctgtgtacagtggccacactgagagaaacctacggcgagatggccgactgctgcgccaagcaagagcccgagagaaacgagtgcttcctgcagcacaaggacgacaaccccaacctgcctagactcgtgcggcctgaagtggacgtgatgtgcaccgccttccacgacaacgaggaaaccttcctgaagaagtacctgtacgagatcgccagacggcacccctacttttacgcccctgagctgctgttcttcgccaagcggtataaggccgccttcaccgagtgttgtcaggccgctgataaggctgcctgcctgctgcctaagctggacgagcttagagacgagggcaaagccagctccgccaagcagagactgaagtgtgccagcctgcagaagttcggcgagagagcctttaaggcctgggccgttgctagactgagccagagatttcccaaggccgagtttgccgaggtgtccaagctcgtgaccgacctgacaaaggtgcacaccgagtgctgccacggcgacctgctggaatgcgccgacgatagagccgacctggccaagtacatctgcgagaaccaggacagcatcagcagcaagctgaaagagtgctgcgagaagcctctgctggaaaagagccactgtatcgccgaggtggaaaacgacgagatgcccgccgatctgccttctctggccgccgattttgtggaaagcaaggacgtgtgcaagaactacgccgaggccaaggacgtgttcctgggcatgtttctgtacgagtacgcccgcagacaccccgactactctgttgtgctgctgctgagactggccaaaacctacgagacaaccctggaaaagtgctgtgccgccgctgatcctcacgagtgttacgccaaggtgttcgacgagttcaagccactggtggaagaaccccagaacctgatcaagcagaactgcgagctgttcgagcagctgggcgagtacaagttccagaacgccctgctcgtgcggtacaccaagaaggtgccccaggtttccacacctacactggttgaggtgtcccggaatctgggcaaagtgggcagcaagtgctgcaagcaccctgaggccaagagaatgccttgcgccgaggattacctgagcgtggtgctgaatcagctgtgcgtgctgcacgagaaaacccctgtgtccgacagagtgaccaagtgctgtaccgagagcctggtcaacagacggccttgctttagcgccctcgaggtggacgagacatacgtgcccaaagagttcaacgccgagacattcaccttccacgccgacatctgtaccctgagcgagaaagagcggcagatcaagaaacagactgccctggtggaactggtcaagcacaagcccaaggccaccaaagaacagctgaaggccgtgatggacgacttcgccgccttcgtggaaaaatgctgcaaggccgacgacaaagagacatgcttcgccgaagagggcaagaaactggtggccgcttctcaggctgccctgggatgataaggtaccgatctttttccctctgccaaaaattatggggacatcatgaagccccttgagcatctgacttctggctaataaaggaaatttattttcattgcaatagtgtgttggaattttttgtgtctctcactcg

SEQ ID NO: 80 - DNA, 2891 bp, synthetic construct
  misc_feature 1..2891: note = synthetic construct
  source 1..2891: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcaagcttgccaccatgattcccgccaaggacatggccaaagtgatgatcgtgatgctggccatctgctttctgaccaagagcgacggcagcgtgtccgagattcagctgatgcacaacctgggcaagcacctgaacagcatggaacgcgtggagtggctgcggaagaaactgcaggacgtgcacaacttcgtggccctgggagcacctctggctcctagagacgccggcagccagcggcctagaaagaaagaggataacgtcctggtcgagagccacgagaagtctctgggcgaagccgacaaggccgacaagacacacacctgtcctccctgtcctgctcccgaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgacaaagaaccaggtgtccctgacctgcctggtcaagggcttctacccttccgatatcgccgtcgagtgggagtccaacggccagccagagaacaactacaagacaacccctcctgtgctggactccgatggctcattcttcctgtacagcaagctgacagtggacaagagccggtggcagcagggcaatgtgttcagctgtagcgtgatgcacgaggccctgcacaaccactacacccagaagtccctgagcctgtctcctggctgatgaggtacctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 81 - DNA, 3041 bp, synthetic construct
  misc_feature 1..3041: note = synthetic construct
  source 1..3041: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccctgcagcgggaattgccaccatggctcacgttcgaggactgcagctgcctggatgtctggctcttgccgctctgtgtagcctggtgcacagccagcacgtgtttctggctcctcagcaagccagatcactgctgcagcgcgtcagaagaagcgtcagcgagatccagctgatgcacaacctgggcaagcacctgaacagcatggaacgcgtggagtggctgcggaagaaactgcaggacgtgcacaacttcgtggccctgggagcacctctggctccaagagacgccggcagccaacggcctagaaagaaagaggacaacgtcctggtcgagagccacgagaagtctctgggcgaagccgataaggctgacggtggcggaggcggaggatctggtggcggtggttctggcggtggcggatctgccgagtctaagtacggacctccttgtcctccctgtcctgctcccgaagctgctggcggcccatccgtgtttctgttccctccaaagcctaaggacaccctgatgatcagcagaacccctgaagtgacctgcgtggtggtggacgtgtcccaagaggatcctgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtcgagtgggagagcaacggccagcctgagaacaactacaagaccacacctcctgtgctggacagcgacggctcattcttcctgtacagccggctgaccgtggacaagagccggtggcaagagggcaacgtgttcagctgctccgtgatgcacgaggccctgcacaaccactacacccagaaaagcctgtctctgagcctgggctgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 82 - DNA, 2909 bp, synthetic construct
  misc_feature 1..2909: note = synthetic construct
  source 1..2909: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcaagcttgccaccatgattcccgccaaggacatggccaaagtgatgatcgtgatgctggccatctgctttctgaccaagagcgacggcaagagcgtgaagaaaagaagcgtgtccgagatccagctgatgcacaacctgggcaagcacctgaacagcatggaacgcgtggagtggctgcggaagaaactgcaggacgtgcacaacttcgtggccctgggagcacctctggctcctagggacgccggcagccagcggcctagaaagaaagaggataacgtcctggtcgagagccacgagaagtctctgggcgaagccgacaaggccgacaaaacacacacctgtcctccctgtcctgctcccgaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtccacaacgccaagaccaagcctagagaggaacagtacaacagcacctacagagtggtgtccgtgctgaccgtgctgcaccaggattggctgaacggcaaagagtacaagtgcaaggtgtctaacaaggccctgcctgctcctatcgagaaaaccatcagcaaggccaagggccagcctagggaaccccaggtttacacactccctccaagcagggacgagctgacaaagaaccaggtgtccctgacctgcctggtcaagggcttctacccttccgatatcgccgtcgagtgggagtccaacggccagccagagaacaactacaagacaacccctcctgtgctggactccgacggctcattcttcctgtacagcaagctgacagtggacaagagccggtggcagcagggcaatgtgttcagctgtagcgtgatgcacgaggccctgcacaaccactacacccagaagtccctgagcctgtctcctggctgatgaggtacctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 83 - DNA, 3983 bp, synthetic construct; identical to SEQ ID NO: 77
  misc_feature 1..3983: note = synthetic construct
  source 1..3983: mol_type = other DNA

SEQ ID NO: 84 - DNA, 1636 bp, synthetic construct
  misc_feature 1..1636: note = synthetic construct
  source 1..1636: mol_type = other DNA
acgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattcgccctgcagcgggaattgccaccatggctcacgttcgaggactgcagctgcctggatgtctggctcttgccgctctgtgtagcctggtgcacagccagcacgtgtttctggctcctcagcaagccagatcactgctgcagcgcgtcagaagaagcgtcagcgagatccagctgatgcacaacctgggcaagcacctgaacagcatggaacgcgtggagtggctgcggaagaaactgcaggacgtgcacaacttcgtggccctgggagcacctctggctccaagagacgccggcagccaacggcctagaaagaaagaggacaacgtcctggtcgagagccacgagaagtctctgggcgaagccgataaggctgacggtggcggaggcggaggatctggtggcggtggttctggcggtggcggatctgccgagtctaagtacggacctccttgtcctccctgtcctgctcccgaagctgctggcggcccatccgtgtttctgttccctccaaagcctaaggacaccctgatgatcagcagaacccctgaagtgacctgcgtggtggtggacgtgtcccaagaggatcctgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtcgagtgggagagcaacggccagcctgagaacaactacaagaccacacctcctgtgctggacagcgacggctcattcttcctgtacagccggctgaccgtggacaagagccggtggcaagagggcaacgtgttcagctgctccgtgatgcacgaggccctgcacaaccactacacccagaaaagcctgtctctgagcctgggctgatgaggtaccgatctttttccctctgccaaaaattatggggacatcatgaagccccttgagcatctgacttctggctaataaaggaaatttattttcattgcaatagtgtgttggaattttttgtgtctctcactcg

SEQ ID NO: 85 - DNA, 1526 bp, synthetic construct
  misc_feature 1..1526: note = synthetic construct
  source 1..1526: mol_type = other DNA
acgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattcaagcttgccaccatgattcccgccaaggacatggccaaagtgatgatcgtgatgctggccatctgctttctgaccaagagcgacggcaagagcgtgaagaaaagaagcgtgtccgagatccagctgatgcacaacctgggcaagcacctgaacagcatggaacgcgtggagtggctgcggaagaaactgcaggacgtgcacaacttcgtggccctgggagcacctctggctcctagggacgccggcagccagcggcctagaaagaaagaggataacgtcctggtcgagagccacgagaagtctctgggcgaagccgacaaggccgacaaaacacacacctgtcctccctgtcctgctcccgaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtccacaacgccaagaccaagcctagagaggaacagtacaacagcacctacagagtggtgtccgtgctgaccgtgctgcaccaggattggctgaacggcaaagagtacaagtgcaaggtgtctaacaaggccctgcctgctcctatcgagaaaaccatcagcaaggccaagggccagcctagggaaccccaggtttacacactccctccaagcagggacgagctgacaaagaaccaggtgtccctgacctgcctggtcaagggcttctacccttccgatatcgccgtcgagtgggagtccaacggccagccagagaacaactacaagacaacccctcctgtgctggactccgacggctcattcttcctgtacagcaagctgacagtggacaagagccggtggcagcagggcaatgtgttcagctgtagcgtgatgcacgaggccctgcacaaccactacacccagaagtccctgagcctgtctcctggctgatgaggtaccgatctttttccctctgccaaaaattatggggacatcatgaagccccttgagcatctgacttctggctaataaaggaaatttattttcattgcaatagtgtgttggaattttttgtgtctctcactcg

SEQ ID NO: 86 - DNA, 2701 bp, synthetic construct
  misc_feature 1..2701: note = synthetic construct
  source 1..2701: mol_type = other DNA
acgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattcgccctgcagcgggaattgccaccatggctcacgttcgaggactgcagctgcctggatgtctggctcttgccgctctgtgtagcctggtgcacagccagcacgtgtttctggctcctcagcaagccagatcactgctgcagcgcgtcagaagaagcgtcagcgagatccagctgatgcacaacctgggcaagcacctgaacagcatggaacgcgtggagtggctgcggaagaaactgcaggacgtgcacaacttcgtggccctgggagcacctctggctccaagagacgccggcagccaacggcctagaaagaaagaggacaacgtcctggtcgagagccacgagaagtctctgggcgaagccgataaggctgacggtggcggaggcggaggatctggtggcggtggttctggcggtggcggatctgacgcccacaaatctgaagtggcccaccggttcaaggacctgggagaagagaacttcaaggccctggtgctgatcgccttcgctcagtacctgcagcagtgccccttcgaggaccacgtgaagctggtcaacgaagtgaccgagttcgccaagacctgcgtggccgacgagagcgccgagaactgtgataagagcctgcacaccctgttcggcgacaagctgtgtacagtggccacactgagagaaacctacggcgagatggccgactgctgcgccaagcaagagcccgagagaaacgagtgcttcctgcagcacaaggacgacaaccccaacctgcctagactcgtgcggcctgaagtggacgtgatgtgcaccgccttccacgacaacgaggaaaccttcctgaagaagtacctgtacgagatcgccagacggcacccctacttttacgcccctgagctgctgttcttcgccaagcggtataaggccgccttcaccgagtgttgtcaggccgctgataaggcagcctgcctgctgcctaagctggacgagcttagagacgagggcaaagccagctccgccaagcagagactgaagtgtgccagcctgcagaagttcggcgagagagcctttaaggcctgggccgttgctagactgagccagagatttcccaaggccgagtttgccgaggtgtccaagctcgtgaccgacctgacaaaggtgcacaccgagtgctgccacggcgacctgctggaatgcgccgacgatagagccgacctggccaagtacatctgcgagaaccaggacagcatcagcagcaagctgaaagagtgctgcgagaagcctctgctggaaaagagccactgtatcgccgaggtggaaaacgacgagatgcccgccgatctgccttctctggccgccgattttgtggaaagcaaggacgtgtgcaagaactacgccgaggccaaggacgtgttcctgggcatgtttctgtacgagtacgcccgcagacaccccgactactctgttgtgctgctgctgagactggccaaaacctacgagacaaccctggaaaagtgctgtgccgccgctgatcctcacgagtgttacgccaaggtgttcgacgagttcaagccactggtggaagaaccccagaacctgatcaagcagaactgcgagctgttcgagcagctgggcgagtacaagttccagaacgccctgctcgtgcggtacaccaagaaggtgccccaggtttccacacctacactggttgaggtgtcccggaatctgggcaaagtgggcagcaagtgctgcaagcaccctgaggccaagagaatgccttgcgccgaggattacctgagcgtggtgctgaatcagctgtgcgtgctgcacgagaaaacccctgtgtccgacagagtgaccaagtgctgtaccgagagcctggtcaacagacggccttgctttagcgccctcgaggtggacgagacatacgtgcccaaagagttcaacgccgagacattcaccttccacgccgacatctgtaccctgagcgagaaagagcggcagatcaagaaacagactgccctggtggaactggtcaagcacaagcccaaggccaccaaagaacagctgaaggccgtgatggacgacttcgccgccttcgtggaaaaatgctgcaaggccgacgacaaagagacatgcttcgccgaagagggcaagaaactggtggccgcttctcaggctgccctgggatgataaggtaccgatctttttccctctgccaaaaattatggggacatcatgaagccccttgagcatctgacttctggctaataaaggaaatttattttcattgcaatagtgtgttggaattttttgtgtctctcactcg

SEQ ID NO: 87 - DNA, 2894 bp, synthetic construct
  misc_feature 1..2894: note = synthetic construct
  source 1..2894: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcaagcttgccaccatgattcccgccaaggacatggccaaagtgatgatcgtgatgctggccatctgctttctgaccaagagcgacggcagcgtgtccgagattcagctgatgcacaacctgggcaagcacctgaacagcatggaacgcgtggagtggctgcggaagaaactgcaggacgtgcacaacttcgtggccctgggagcacctctggctcctagagacgccggcagccagcggcctagaaagaaagaggataacgtcctggtcgagagccacgagaagtctctgggcgaagccgacaaggccgacgacaagacacacacctgtcctccctgtcctgctcccgaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgacaaagaaccaggtgtccctgacctgcctggtcaagggcttctacccttccgatatcgccgtcgagtgggagtccaacggccagccagagaacaactacaagacaacccctcctgtgctggactccgatggctcattcttcctgtacagcaagctgacagtggacaagagccggtggcagcagggcaatgtgttcagctgtagcgtgatgcacgaggccctgcacaaccactacacccagaagtccctgagcctgtctcctggctgatgaggtacctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 88 - DNA, 2912 bp, synthetic construct
  misc_feature 1..2912: note = synthetic construct
  source 1..2912: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcaagcttgccaccatgattcccgccaaggacatggccaaagtgatgatcgtgatgctggccatctgctttctgaccaagagcgacggcaagagcgtgaagaaaagaagcgtgtccgagatccagctgatgcacaacctgggcaagcacctgaacagcatggaacgcgtggagtggctgcggaagaaactgcaggacgtgcacaacttcgtggccctgggagcacctctggctcctagagacgccggcagccagcggcctagaaagaaagaggataacgtcctggtcgagagccacgagaagtctctgggcgaagccgacaaggccgacgacaagacacacacctgtcctccctgtcctgctcccgaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgacaaagaaccaggtgtccctgacctgcctggtcaagggcttctacccttccgatatcgccgtcgagtgggagtccaacggccagccagagaacaactacaagacaacccctcctgtgctggactccgacggctcattcttcctgtacagcaagctgacagtggacaagagccggtggcagcagggcaatgtgttcagctgtagcgtgatgcacgaggccctgcacaaccactacacccagaagtccctgagcctgtctcctggctgatgaggtacctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 89 - DNA, 2897 bp, synthetic construct
  misc_feature 1..2897: note = synthetic construct
  source 1..2897: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcaagcttgccaccatgattcccgccaaggacatggccaaagtgatgatcgtgatgctggccatctgctttctgaccaagagcgacggcagcgtgtccgagattcagctgatgcacaacctgggcaagcacctgaacagcatggaacgcgtggagtggctgcggaagaaactgcaggacgtgcacaacttcgtggccctgggagcacctctggctcctagagacgccggcagccagcggcctagaaagaaagaggataacgtcctggtcgagagccacgagaagtctctgggcgaagccgacaaggccgacgtggacaagacacacacctgtcctccctgtcctgctcccgaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgacaaagaaccaggtgtccctgacctgcctggtcaagggcttctacccttccgatatcgccgtcgagtgggagtccaacggccagccagagaacaactacaagacaacccctcctgtgctggactccgatggctcattcttcctgtacagcaagctgacagtggacaagagccggtggcagcagggcaatgtgttcagctgtagcgtgatgcacgaggccctgcacaaccactacacccagaagtccctgagcctgtctcctggctgatgaggtacctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 90 - DNA, 2915 bp, synthetic construct
  misc_feature 1..2915: note = synthetic construct
  source 1..2915: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcaagcttgccaccatgattcccgccaaggacatggccaaagtgatgatcgtgatgctggccatctgctttctgaccaagagcgacggcaagagcgtgaagaaaagaagcgtgtccgagatccagctgatgcacaacctgggcaagcacctgaacagcatggaacgcgtggagtggctgcggaagaaactgcaggacgtgcacaacttcgtggccctgggagcacctctggctcctagagacgccggcagccagcggcctagaaagaaagaggataacgtcctggtcgagagccacgagaagtctctgggcgaagccgacaaggccgacgtggacaagacacacacctgtcctccctgtcctgctcccgaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgacaaagaaccaggtgtccctgacctgcctggtcaagggcttctacccttccgatatcgccgtcgagtgggagtccaacggccagccagagaacaactacaagacaacccctcctgtgctggactccgacggctcattcttcctgtacagcaagctgacagtggacaagagccggtggcagcagggcaatgtgttcagctgtagcgtgatgcacgaggccctgcacaaccactacacccagaagtccctgagcctgtctcctggctgatgaggtacctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 91 - DNA, 2285 bp, synthetic construct
  misc_feature 1..2285: note = synthetic construct
  source 1..2285: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccttgccctgcagcgggaattgccaccatgtacaagatgcagctgctgagctgtatcgccctgacactggtgctggtggccaactccaagagcgtgaagaaaagaagcgtgtccgagatccagctgatgcacaacctgggcaagcacctgaacagcatggaacgcgtggagtggctgcggaagaaactgcaggacgtgcacaacttcgtggccctgggagcacctctggctcctagagacgccggcagccagcggcctagaaagaaagaggataacgtcctggtcgagagccacgagaagtctctgggcgaagccgataaggccgacgtgaacgtgctgaccaaggccaagagccagtgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 92 - DNA, 2300 bp, synthetic construct
  misc_feature 1..2300: note = synthetic construct
  source 1..2300: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccttgccctgcagcgggaattgccaccatgattcccgccaaggacatggccaaagtgatgatcgtgatgctggccatctgctttctgaccaagagcgacggcaagagcgtgaagaaaagaagcgtgtccgagatccagctgatgcacaacctgggcaagcacctgaacagcatggaacgcgtggagtggctgcggaagaaactgcaggacgtgcacaacttcgtggccctgggagcacctctggctcctagagacgccggcagccagcggcctagaaagaaagaggataacgtcctggtcgagagccacgagaagtctctgggcgaagccgataaggccgacgtgaacgtgctgaccaaggccaagagccagtgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 93 - DNA, 3071 bp, synthetic construct
  misc_feature 1..3071: note = synthetic construct
  source 1..3071: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccctgcagcgggaattgccaccatggctcacgttcgaggactgcagctgcctggatgtctggctcttgccgctctgtgtagcctggtgcacagccagcacgtgtttctggctcctcagcaagccagatcactgctgcagcgcgtcagaagaagcgtcagcgagatccagctgatgcacaacctgggcaagcacctgaacagcatggaacgcgtggagtggctgcggaagaaactgcaggacgtgcacaacttcgtggccctgggagcacctctggctccaagagacgccggcagccaacggcctagaaagaaagaggacaacgtcctggtcgagagccacgagaagtctctgggcgaagccgataaggccgacgtgaacgtgctgaccaaggccaagtctcaaggtggcggaggcggaggatctggtggtggtggatctggcggcggaggttctgccgagtctaagtacggacctccttgtcctccctgtcctgctcccgaagctgctggcggcccatccgtgtttctgttccctccaaagcctaaggacaccctgatgatcagcagaacccctgaagtgacctgcgtggtggtcgacgtgtcccaagaggatcctgaggtgcagttcaattggtacgtggacggcgtggaagtgcacaacgccaagaccaagcctagagaggaacagttcaacagcacctacagagtggtgtctgtgctgaccgtgctgcaccaggattggctgaacggcaaagagtacaagtgcaaggtgtccaacaagggcctgcctagcagcatcgagaaaaccatctctaaggccaagggccagcctcgcgaaccccaggtttacacactgcctccaagccaagaggaaatgaccaagaaccaggtgtccctgacctgcctggtcaagggcttctacccttccgatatcgccgtcgagtgggagagcaacggccagcctgagaacaactacaagaccacacctcctgtgctggacagcgacggctcattcttcctgtacagccggctgaccgtggacaagagcaggtggcaagagggcaacgtgttcagctgctccgtgatgcacgaggccctgcacaaccactacacccagaaaagcctgtctctgagcctgggctgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 94 - DNA, 4136 bp, synthetic construct
  misc_feature 1..4136: note = synthetic construct
  source 1..4136: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccctgcagcgggaattgccaccatggctcacgttcgaggactgcagctgcctggatgtctggctcttgccgctctgtgtagcctggtgcacagccagcacgtgtttctggctcctcagcaagccagatcactgctgcagcgcgtcagaagaagcgtcagcgagatccagctgatgcacaacctgggcaagcacctgaacagcatggaacgcgtggagtggctgcggaagaaactgcaggacgtgcacaacttcgtggccctgggagcacctctggctccaagagacgccggcagccaacggcctagaaagaaagaggacaacgtcctggtcgagagccacgagaagtctctgggcgaagccgataaggccgacgtgaacgtgctgaccaaggccaagtctcaaggtggcggaggcggaggatctggtggtggtggatctggcggcggaggttctgacgcccacaaatctgaagtggcccaccggttcaaggacctgggagaagagaacttcaaggccctggtgctgatcgccttcgctcagtacctgcagcagtgccccttcgaggaccacgtgaagctggtcaacgaagtgaccgagttcgccaagacctgcgtggccgacgagagcgccgagaactgtgataagagcctgcacaccctgttcggcgacaagctgtgtacagtggccacactgagagaaacctacggcgagatggccgactgctgcgccaagcaagagcccgagagaaacgagtgcttcctgcagcacaaggacgacaaccccaacctgcctagactcgtgcggcctgaagtggacgtgatgtgcaccgccttccacgacaacgaggaaaccttcctgaagaagtacctgtacgagatcgccagacggcacccctacttttacgcccctgagctgctgttcttcgccaagcggtataaggccgccttcaccgagtgttgtcaggccgctgataaggctgcctgcctgctgcctaagctggacgagcttagagacgagggcaaagccagctccgccaagcagagactgaagtgtgccagcctgcagaagttcggcgagagagcctttaaggcctgggccgttgctagactgagccagagatttcccaaggccgagtttgccgaggtgtccaagctcgtgaccgacctgacaaaggtgcacaccgagtgctgccacggcgacctgctggaatgcgccgacgatagagccgacctggccaagtacatctgcgagaaccaggacagcatcagcagcaagctgaaagagtgctgcgagaagcctctgctggaaaagagccactgtatcgccgaggtggaaaacgacgagatgcccgccgatctgccttctctggccgccgattttgtggaaagcaaggacgtgtgcaagaactacgccgaggccaaggacgtgttcctgggcatgtttctgtacgagtacgcccgcagacaccccgactactctgttgtgctgctgctgagactggccaaaacctacgagacaaccctggaaaagtgctgtgccgccgctgatcctcacgagtgttacgccaaggtgttcgacgagttcaagccactggtggaagaaccccagaacctgatcaagcagaactgcgagctgttcgagcagctgggcgagtacaagttccagaacgccctgctcgtgcggtacaccaagaaggtgccccaggtttccacacctacactggttgaggtgtcccggaatctgggcaaagtgggcagcaagtgctgcaagcaccctgaggccaagagaatgccttgcgccgaggattacctgagcgtggtgctgaatcagctgtgcgtgctgcacgagaaaacccctgtgtccgacagagtgaccaagtgctgtaccgagagcctggtcaacagacggccttgctttagcgccctcgaggtggacgagacatacgtgcccaaagagttcaacgccgagacattcaccttccacgccgacatctgtaccctgagcgagaaagagcggcagatcaagaaacagactgccctggtggaactggtcaagcacaagcccaaggccaccaaagaacagctgaaggccgtgatggacgacttcgccgccttcgtggaaaaatgctgcaaggccgacgacaaagagacatgcttcgccgaagagggcaagaaactggtggccgcttctcaggctgccctgggatgataaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 95 - DNA, 4383 bp, synthetic construct
  misc_feature 1..4383: note = synthetic construct
  source 1..4383: mol_type = other DNA
acgcgttcgagctcgccccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgggggatcttggtggcgtgaaactcccgcagatcttcggccagcgaattccagaagccaccatggactatcctgctgccaagagggtcaagttggactctagaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcctccaagcctgggacctctattatcatgtgttccgacgaatctcaaagactagagatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctagtaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggactatcctgctgccaagagggtcaagttggactctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagaggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagaggcgttcaggtggaaaccatcagtccaggggatggccgaacttttccaaagagagggcagacttgcgtcgtgcattatactggtatgctggaggatgggaaaaagttcgactcttccagagatcggaacaaaccattcaaattcatgctcgggaaacaggaagttatccgcggatgggaggagggcgtggcccagatgtccgtgggccagcgcgccaagctaaccatctccccagactacgcctacggagccaccggacaccccggtatcatacccccacacgccacccttgtgtttgacgtggaactgcttaagctagagactagaggcgtgcaggtcgagaccatcagccccggcgacggccgcacctttcccaagagaggccagacttgcgtggtccactacaccggcatgctggaggacggcaagaagttcgacagcagccgcgaccgcaacaagcccttcaagttcatgctgggcaaacaggaagtgatccgcggctgggaggaaggcgtggctcagatgagcgtggggcagcgggccaagctgaccatcagccccgactatgcctacggcgccaccggccaccccggcatcatccccccccacgccaccctggtgttcgacgtggagctgctgaagctggagtgataaggatccccactccagtgcccaccagccttgtcctaataaaattaagttgcatcattttgtctgactaggtgtccttctataatattatggggtggaggggggtggtttggagcaaggcgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattgccaccatgtacaagatgcagctgctgagctgtatcgccctgacactggtgctggtggccaactccaagagcgtgaagaaaagaagcgtgtccgagatccagctgatgcacaacctgggcaagcacctgaacagcatggaacgcgtggagtggctgcggaagaaactgcaggacgtgcacaacttcgtggccctgggagcacctctggctcctagagacgccggcagccagcggcctagaaagaaagaggataacgtcctggtcgagagccacgagaagtctctgggcgaagccgataaggccgacgtgaacgtgctgaccaaggccaagagccagtgatgaggtaccgatctttttccctctgccaaaaattatggggacatcatgaagccccttgagcatctgacttctggctaataaaggaaatttattttcattgcaatagtgtgttggaattttttgtgtctctcactcggaa

SEQ ID NO: 96 - DNA, 4398 bp, synthetic construct
  misc_feature 1..4398: note = synthetic construct
  source 1..4398: mol_type = other DNA
acgcgttcgagctcgccccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgggggatcttggtggcgtgaaactcccgcagatcttcggccagcgaattccagaagccaccatggactatcctgctgccaagagggtcaagttggactctagaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcctccaagcctgggacctctattatcatgtgttccgacgaatctcaaagactagagatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctagtaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggactatcctgctgccaagagggtcaagttggactctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagaggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagaggcgttcaggtggaaaccatcagtccaggggatggccgaacttttccaaagagagggcagacttgcgtcgtgcattatactggtatgctggaggatgggaaaaagttcgactcttccagagatcggaacaaaccattcaaattcatgctcgggaaacaggaagttatccgcggatgggaggagggcgtggcccagatgtccgtgggccagcgcgccaagctaaccatctccccagactacgcctacggagccaccggacaccccggtatcatacccccacacgccacccttgtgtttgacgtggaactgcttaagctagagactagaggcgtgcaggtcgagaccatcagccccggcgacggccgcacctttcccaagagaggccagacttgcgtggtccactacaccggcatgctggaggacggcaagaagttcgacagcagccgcgaccgcaacaagcccttcaagttcatgctgggcaaacaggaagtgatccgcggctgggaggaaggcgtggctcagatgagcgtggggcagcgggccaagctgaccatcagccccgactatgcctacggcgccaccggccaccccggcatcatccccccccacgccaccctggtgttcgacgtggagctgctgaagctggagtgataaggatccccactccagtgcccaccagccttgtcctaataaaattaagttgcatcattttgtctgactaggtgtccttctataatattatggggtggaggggggtggtttggagcaaggcgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattgccaccatgattcccgccaaggacatggccaaagtgatgatcgtgatgctggccatctgctttctgaccaagagcgacggcaagagcgtgaagaaaagaagcgtgtccgagatccagctgatgcacaacctgggcaagcacctgaacagcatggaacgcgtggagtggctgcggaagaaactgcaggacgtgcacaacttcgtggccctgggagcacctctggctcctagagacgccggcagccagcggcctagaaagaaagaggataacgtcctggtcgagagccacgagaagtctctgggcgaagccgataaggccgacgtgaacgtgctgaccaaggccaagagccagtgatgaggtaccgatctttttccctctgccaaaaattatggggacatcatgaagccccttgagcatctgacttctggctaataaaggaaatttattttcattgcaatagtgtgttggaattttttgtgtctctcactcggaa

SEQ ID NO: 97 - DNA, 904 bp, synthetic construct
  misc_feature 1..904: note = synthetic construct
  source 1..904: mol_type = other DNA
acgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattcgcccttttccttgccctgcagcgggaattgccaccatgattcccgccaaggacatggccaaagtgatgatcgtgatgctggccatctgctttctgaccaagagcgacggcaagagcgtgaagaaaagaagcgtgtccgagatccagctgatgcacaacctgggcaagcacctgaacagcatggaacgcgtggagtggctgcggaagaaactgcaggacgtgcacaacttcgtggccctgggagcacctctggctcctagagacgccggcagccagcggcctagaaagaaagaggataacgtcctggtcgagagccacgagaagtctctgggcgaagccgataaggccgacgtgaacgtgctgaccaaggccaagagccagtgatgaggtaccgatctttttccctctgccaaaaattatggggacatcatgaagccccttgagcatctgacttctggctaataaaggaaatttattttcattgcaatagtgtgttggaattttttgtgtctctcactcg

SEQ ID NO: 98 - DNA, 2211 bp, Adeno-Associated Virus hu68
  source 1..2211: mol_type = unassigned DNA
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 99 - DNA, 2211 bp, Adeno-Associated Virus hu68
  source 1..2211: mol_type = unassigned DNA
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgacggcagtcaggcggtgggccgctcatccttctactgcctggagtacttcccttcgcagatgctgaggactggcaacaacttccagttcagctacgagttcgagaacgtccctttccacagcagctacgcccacagccagagtttggaccgcttgatgaaccctctgatcgaccagtacctgtactacctgtcaaagacgatcaacggttctggccagaaccagcagacgctgaagttcagcgtggccgggcctagcaacatggccgtccagggcagaaactacatccctgggcccagctaccggcagcagagagtctcaaccactgtgactcagaacaacaacagtgagttcgcctggcctggcgccagctcttgggccctcaacggccgcaactcgctgatgaacccaggcccagccatggccagtcacaaggagggcgaggaccgtttcttccctttgtctggctctctgatcttcggcaagcaggggaccggcagagacaacgtggacgcggacaaggtcatgatcacgaacgaggaggagatcaagaccaccaaccctgtggcaaccgagtcctacggccaggtggcaaccaaccaccagagcgcccaggcacaggcgcagactggctgggtccagaaccaggggatcctgcctggcatggtgtggcaggaccgtgacgtgtacctgcagggccctatctgggcaaagatccctcacacggacggcaacttccacccttctcctctgatgggcggcttcggcatgaagcacccgcctcctcagatcctcatcaagaacactccggtcccggcagaccctccgacggccttcaacaaggacaagctgaactcattcatcactcagtactccactggccaggtcagcgtggagatcgagtgggagctgcagaaggagaacagcaagcgttggaacccagagatccagtacacttccaactactacaagtctaacaacgtggagttcgccgtcaacactgagggtgtgtacagtgagcctcgccctatcggcacccggtacctcacccgaaacttgtga

SEQ ID NO: 100 - protein, 736 aa, Adeno-Associated Virus hu68
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

(19) United States
(12) Patent Application Publication
(10) Pub. No.: US 2025/0186612 A1
(43) Pub. Date: Jun. 12, 2025

(54) VIRAL VECTORS ENCODING PARATHYROID HORMONE FUSIONS AND USES THEREOF IN TREATING HYPOPARATHYROIDISM

(71) Applicant: The Trustees of the University of Pennsylvania, Philadelphia, PA (US)

(72) Inventors: James M. Wilson, Philadelphia, PA (US); Christian Hinderer, Baltimore, MD (US); Makoto Horiuchi, Wallingford, PA (US)

(73) Assignee: The Trustees of the University of Pennsylvania, Philadelphia, PA (US)

(21) Appl. No.: 18/843,444

(22) PCT Filed: Mar. 3, 2023
(86) PCT No.: PCT/US2023/063679

§ 371 Date: Sep. 3, 2024

Related U.S. Application Data
(60) Provisional application No. 63/316,215, filed on Mar. 3, 2022.

(51) Int. Cl. A61K 48/00; A61K 9/00; A61K 38/17; A61K 38/29; A61P 5/18; C12N 15/86

(52) U.S. Cl. CPC A61K 48/005; A61K 9/0019; A61K 38/1774; A61K 38/29; A61K 48/0075; A61K 48/0083; A61P 5/18; C12N 15/86; C12N 2750/14143; C12N 2830/002

(57) ABSTRACT

Compositions and methods for treating hypoparathyroidism in a subject are provided. A viral vector is provided which includes a nucleic acid molecule comprising a sequence encoding a PTH fusion protein and regulatory sequences which direct expression thereof.

### REFERENCE TO AN ELECTRONIC SEQUENCE LISTING

The contents of the electronic sequence listing (22-10017.PCT_Seq-Listing.xml; Size: 304 kb: and Date of Creation: Mar. 1, 2023) is herein incorporated by reference in its entirety.

### BACKGROUND OF THE INVENTION

Parathyroid hormone (PTH) is an 84 amino acid hormone produced by the parathyroid glands. PTH regulates serum calcium concentration through its effects on bone, kidney, and intestine. Hypoparathyroidism (HP) is a rare condition in which PTH fails to be secreted from the parathyroid glands. HP causes hypocalcemia that leads to further symptoms including weakness, muscle cramps, headaches, and/or uncontrollable twitching and cramping spasms of muscles. Recombinant human PTH (rhPTH(1-84)) was approved by FDA as a treatment for adult chronic HP, rhPTH(1-84) needs to be subcutaneously injected daily because PTH has a short serum half-life. While rhPTH(1-84) showed a good safety profile in clinical studies, exogenous PTH increases a risk of osteosarcoma in rats suggesting a safety concem on the long-term continuous use of rhPTH(1-84).

What is needed are improved therapies for hypoparathyroidism.

### SUMMARY OF THE INVENTION

Viral vectors encoding parathyroid hormone (PTH) fusion protein constructs are provided herein. These viral vectors may achieve, in some embodiments, sustained expression of the PTH fusion in subjects and/or increased circulating half-life, as compared to vector-mediated delivery of a PTH fusion without a fusion partner. Further provided are methods of making and using such viral vectors.

In one aspect, a viral vector is provided which includes a nucleic acid comprising a polynucleotide sequence encoding a fusion protein. The fusion protein includes (a) a leader sequence comprising a secretion signal peptide and a propeptide, (b) a parathyroid hormone (PTH) fusion, and (c) a fusion domain comprising either (i) an IgG Fc or a functional variant thereof or (ii) an albumin or a functional variant thereof. In one embodiment, the vector is an adeno-associated viral vector.

In one embodiment, the (i) the secretion signal peptide of the leader sequence comprises a thrombin signal peptide; (ii) the leader sequence comprises a thrombin propeptide; and/or (iii) the leader sequence comprises a thrombin leader sequence. In another embodiment, the leader sequence comprises an IL-2 leader sequence in combination with a PTH propeptide or thrombin propeptide. In one embodiment, the PTH fusion is selected from SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 39, SEQ ID NO: 42, SEQ ID NO: 44, SEQ ID NO: 46, SEQ ID NO: and 48 and functional variants thereof.

In one embodiment, the fusion domain is a human IgG4 Fc having the sequence of

SEQ ID NO: 6, or a sequence sharing at least 90% identity therewith, or a functional variant thereof. In another embodiment, the fusion domain is a human albumin having the sequence of SEQ ID NO: 9, or a sequence sharing at least 90% identity therewith, or a functional variant thereof. In one embodiment, the fusion domain is a rhesus IgG4 Fc having the sequence of SEQ ID NO: 8, or a sequence sharing at least 90% identity therewith, or a functional variant thereof. In one embodiment, the fusion domain is a human IgG1 Fc having the sequence of SEQ ID NO: 7, or a sequence sharing at least 90% identity therewith, or a functional variant thereof.

In another aspect, the viral vector includes an AAV capsid, and a vector genome packaged in the AAV capsid, said vector genome comprising AAV inverted terminal repeats (ITRs), the polynucleotide sequence encoding the fusion protein, and regulatory sequences which direct expression of the fusion protein.

In another aspect, a pharmaceutical composition suitable for use in treating hypoparathyroidism in a subject is provided. The composition includes an aqueous liquid and the viral vector as described herein. In one embodiment, the subject is a human.

In vet another aspect, use of a viral vector as described herein is provided for the manufacture of a medicament for treating a subject having hypoparathyroidism, optionally diabetes.

In another aspect, a method of treating a subject having hypoparathyroidism is provided. The method includes administering to the subject an effective amount of a viral vector or composition as described herein,

Other aspects and advantages of the invention will be readily apparent from the following detailed description of the invention.

### BRIEF DESCRIPTION OF THE DRAWINGS

FIG. 1A is a schematic showing an overview of PTH constructs described herein that utilize heterologous leaders (signal peptides and propeptides) in combination with mature PTH.

FIG. 1B shows a table showing experimental outline for an experiment described in Example 3.

FIG. 1C shows results of the experiment of FIG. 1B. Briefly, three groups of RagKO mice were treated with vector utilizing native leader or IL2 signal (in combination with PTH propeptide), or vehicle. Plasma PTH levels (day 14) and serum calcium levels (day 28) were comparable for both vectors.

FIG. 2A and FIG. 2B show schematic of PTH constructs described herein that utilize fusion partners in combination with truncated PTH.

FIG. 2C shows in vitro expression in HEK293 cells of the constructs described in FIG. 2B.

FIG. 3A is a gel showing stained PTH Fc fusion proteins produced from described constructs and purified using protein A column purification. Fractions 3 and 4 produced under reduced (R) and non-reduced (NR) conditions are shown with ladder.

FIG. 3B is a graph showing the results of a potency assay from purified proteins of FIG. 3A, purified PTH73 Fc fusion of fractions 3 and 4 showed similar potency to PTH34 (active portion of protein).

FIG. 3C is a graph showing hPTH73 Fc fusion levels produced from inducible constructs in vitro when induced using 40 nM rapamycin.

FIG. 4A shows a table showing experimental outline for an experiment described in Example 3.

FIG. 4B shows results of the experiment of FIG. 4A. Briefly, three groups of RagKO mice were treated with vehicle, vector utilizing native leader (hPTH(N)) or native leader with Fc fusion (hPTH73.P.Fc). Plasma hPTH potency was determined from day 14 and day 28 bleeds.

FIG. 5A shows a table showing experimental outline for an experiment described in Example 3. Briefly, three groups of RagKO mice were treated with vehicle, inducible vector utilizing native leader with Fc fusion. Rapamycin was administered at day 12 for groups 2 and 3 and again at day 38 for group 3.

FIG. 5B shows plasma hPTH potency determined from day 14 and day 38 bleeds.

FIG. 5C shows serum calcium levels.

FIG. 6 shows a human PTH full length sequence as provided in SEQ ID NO: 1.

FIG. 7A shows the study design of the experiment described in Example 4. FIG. 7B shows preliminary data, including the NAB titer for 5 different AAV capsids.

FIGS. 8A-8C show the results of the experiment described in Example 5. FIG. 8A shows body weights for animals treated with the noted vectors and dosages. FIG. 8B shows serum calcium for animals treated with the noted vectors and dosages. FIG. 8C shows plasma hPTH levels for animals treated with the noted vectors and dosages.

FIG. 9A-8B show the results of the experiment described in Example 6. FIG. 9A shows plasma hPTH levels for animals treated with the noted vectors and dosages. FIG. 9B shows serum calcium for animals treated with the noted vectors and dosages.

### DETAILED DESCRIPTION OF THE INVENTION

Described herein are adeno-associated viral (AAV) vectors expressing PTH fusion proteins to treat hypoparathyroidism in patients with a single intramuscular vector administration. Delivery of these constructs to subjects in need thereof via a number of routes, and particularly by expression in vivo mediated by a recombinant vector such as a rAAV vector, is described. Also provided are methods of using these constructs in regimens for treating HP in a subject in need thereof and increasing the half-life of PTH in a subject. In addition, methods are provided for enhancing the activity of PTH in a subject.

### PTH Fusion Proteins

Hypoparathyroidism (HP) is characterized by low serum calcium levels caused by an insufficient secretion of parathyroid hormone (PTH). Despite normalization of serum calcium levels by treatment with activated vitamin D proteins and calcium supplementation, patients are suffering from impaired quality of life and are at increased risk for a number of comorbidities. In a number of recent studies, replacement therapy with recombinant human PTH (rhPTH(1-84)) as well as therapy with the N-terminal PTH fragment (rhPTH(1-34)) have been investigated. Both drugs have been shown to normalize serum calcium while reducing needs for activated vitamin D and calcium supplements. However, once-a-day injections cause large fluctuations in serum calcium. Twice-a-day injections diminish fluctuations, but don't restore the normal physiology of calcium homeostasis.

Parathyroid hormone (PTH) is a single-chain polypeptide composed of 115 amino acids having the following sequence:

(SEQ ID NO: 1)
MIPAKDMAKV MIVMLAICFL TKSDGKSVKK RSVSEIQLMH
NLGKHLNSMERVEWLRKKLQ DVHNFVALGA PLAPRDAGSQ
RPRKKEDNVL VESHEKSLGEADKADVNVLT KAKSQ

This full-length sequence includes the signal peptide (amino acids 1-25) as well as the propeptide (amino acids 26-31). The mature protein consists of amino acids 32-115 of SEQ ID NO: 1—SVSEIQLMHNLGKHLNSMERVEWLRKKLQDVHNFVALGAPLAPRDAGSQRPRKKE DNVLVESHEKSLGEADKADVNVLTKAKSQ (SEQ ID NO: 2).

The disclosure provides fusion proteins comprising one or more copies of a PTH, as well as polynucleotides and vectors encoding such fusion proteins. As used herein, “a PTH” or “a PTH protein” refers to the full-length pre-proparathyroid hormone (including the signal sequence and propeptide), a sequence encompassing the propeptide and mature peptide (e.g., amino acids about 26-115), the mature peptide (e.g., amino acids about 32-115), peptides with C-terminal or N-terminal truncations. In particular, peptides including amino acids 1-33, 1-34, 1-72, 1-73, 1-74, 1-75 of the mature peptide sequence (SEQ ID NO: 2) are useful herein. These truncated peptides may be combined with the native PTH propeptide and signal peptide, or a heterologous propeptide and/or signal peptide. A number of constructs are described herein that embody various combinations of these sequences. As used herein, reference to PTH and a number following (e.g., PTHX, PTH34, PTH73) refers to a shortened PTH peptide that includes the first X number of peptides starting from the N-terminus of SEQ ID NO: 2. For example, PTH34 includes about the first 34 amino acids of the mature peptide, while PTH73 includes about the first 73 amino acids of the mature peptide.

As used herein, the term “leader sequence” refers to any N-terminal sequence of the PTH polypeptide. In some embodiments, the leader includes a secretion signal peptide, a propeptide, or both.

In some embodiments, the fusion protein comprises a polynucleotide sequence encoding a fusion protein comprising (a) a leader sequence comprising a secretion signal peptide, and optionally, a propeptide, (b) a PTH, and optionally (c) a fusion domain. In one embodiment, the PTH fusion comprises a thrombin leader sequence, a PTH protein, and an IgG Fc or functional variant thereof. In another embodiment, the fusion protein comprises a thrombin leader, a PTH protein, and an albumin or functional variant thereof. In another embodiment, the PTH fusion comprises a PTH leader sequence, a PTH protein, and an IgG Fc or functional variant thereof. In another embodiment, the fusion protein comprises a PTH leader, a PTH protein, and an albumin or functional variant thereof. In another embodiment, the PTH fusion comprises an IL2 signal sequence, a PTH propeptide, a PTH protein, and an IgG Fc or functional variant thereof. In another embodiment, the fusion protein comprises an IL2 signal sequence, a PTH propeptide, a PTH protein, and an albumin or functional variant thereof.

In some embodiments, PTH fusions include variants which may include up to about 10% variation from a PTH nucleic acid or amino acid sequence described herein or known in the art, which retain the function of the wild type sequence. As used herein, by “retain function” it is meant that the nucleic acid or amino acid functions in the same way as the wild type sequence, although not necessarily at the same level of expression or activity. For example, in one embodiment, a functional variant has increased expression or activity as compared to the wild type sequence. In another embodiment, the functional variant has decreased expression or activity as compared to the wild type sequence. In one embodiment, the functional variant has 10%, 20%, 30%, 40%, 50%, 60%, 70%, 80%, 90% or greater increase or decrease in expression or activity as compared to the wild type sequence. In some embodiments, the functional variant has 1. 2, 3, 4, 5, 6, 7, 8, 9, or 10 amino acid changes as compared to the wild type sequence.

The fusion comprises, in one embodiment, a PTH protein in combination with heterologous sequences. The PTH protein shares at least 90%, 95%, 97%, 98%, 99% or 100% identity with native human PTH (SEQ ID NO: 1 or 2). In one embodiment, the PTH protein has at most 1, 2, or 3 amino acid substitutions as compared to the native sequence. In another embodiment, the PTH sequence is derived from a species other than human. For example, the PTH may be from a non-human primate, dog, cat, mouse, rat, sheep, cow, horse, etc. In some embodiments, it is desirable to alter the native PTH sequence to optimize one or more features thereof. In one embodiment, the PTH protein has a sequence comprising, or consisting of, SEQ ID NO: 1. In one embodiment, the variant shares at least 90% identity, 95% identity, 97% identity, 98% identity, 99% identity or 100% identity with SEQ ID NO: 1. In one embodiment, the PTH protein has a sequence comprising, or consisting of, SEQ ID NO: 2. In one embodiment, the variant shares at least 90% identity, 95% identity, 97% identity, 98% identity, 99% identity or 100% identity with SEQ ID NO: 2.

The leader sequence may be derived from the same species for which administration is ultimately intended, e.g., a human. As used herein, the terms “derived” or “derived from” mean the sequence or protein is sourced from a specific subject species or shares the same sequence as a protein or sequence sourced from a specific subject species. For example, a leader sequence which is “derived from” a human, shares the same sequence (or a variant thereof, as defined herein) as the same leader sequence as expressed in a human. However, the specified nucleic acid or amino acid need not actually be sourced from a human. Various techniques are known in the art which are able to produce a desired sequence, including mutagenesis of a similar protein (e.g., a homolog) or artificial production of a nucleic acid or amino acid sequence. The “derived” nucleic acid or amino acid retains the function of the same nucleic acid or amino acid in the species from which it is “derived”, regardless of actual source of the derived sequence.

The term “amino acid substitution” and its synonyms are intended to encompass modification of an amino acid sequence by replacement of an amino acid with another, substituting, amino acid. The substitution may be a conservative substitution. It may also be a non-conservative substitution. The term conservative, in referring to two amino acids, is intended to mean that the amino acids share a common property recognized by one of skill in the art. For example, amino acids having hydrophobic nonacidic side chains, amino acids having hydrophobic acidic side chains, amino acids having hydrophilic nonacidic side chains, amino acids having hydrophilic acidic side chains, and amino acids having hydrophilic basic side chains. Common properties may also be amino acids having hydrophobic side chains, amino acids having aliphatic hydrophobic side chains, amino acids having aromatic hydrophobic side chains, amino acids with polar neutral side chains, amino acids with electrically charged side chains, amino acids with electrically charged acidic side chains, and amino acids with electrically charged basic side chains. Both naturally occurring and non-naturally occurring amino acids are known in the art and may be used as substituting amino acids in embodiments. Methods for replacing an amino acid are well known to the skilled in the art and include, but are not limited to, mutations of the nucleotide sequence encoding the amino acid sequence. Reference to “one or more” herein is intended to encompass the individual embodiments of, for example, 1, 2, 3, 4, 5, 6, or more.

In one embodiment, the leader is a human thrombin (Factor II) sequence. In one embodiment, the thrombin leader has the sequence shown in SEQ ID NO: 3: MAHVRGLQLPGCLALAALCSLVHSQHVFLAPQQARSLLQRVRR, or a functional variant thereof having at most 1, 2, or 3 amino acid substitutions.

In another embodiment, the leader is a human IL2 sequence. In one embodiment, the IL2 leader has the sequence shown in SEQ ID NO: 4: MYKMQLLSCIALTLVLVANS or a functional variant thereof having at most 1, 2, or 3 amino acid substitutions. In certain embodiments, the leader includes a propeptide (e.g., from human PTH, human thrombin or other sequence) in addition to the IL2 signal peptide.

In another embodiment, the leader is a human PTH sequence. In one embodiment, the PTH leader has the sequence shown in SEQ ID NO: 5: MIPAKDMAKVMIVMLAICFLTKSDGKSVKKR or a functional variant thereof having at most 1, 2, or 3 amino acid substitutions.

In one embodiment, functional variants of the desired leader include variants which may include up to about 10% variation from a leader nucleic acid or amino acid sequence described herein or known in the art, which retain the function of the wild-type sequence.

In some embodiments, the coding regions for both the leader and PTH peptide are incorporated into a single nucleic acid sequence without a linker between the coding sequences of the leader and PTH.

In certain embodiments, the fusion protein further includes a fusion domain. The fusion domain, in one embodiment, is a human IgG Fc fragment or a functional variant thereof. Immunoglobulins typically have long circulating half-lives in vivo. By fusing the PTH protein (and leader) to an IgG Fc, the circulation time of the fusion protein is prolonged, while the function of the PTH is preserved. In another embodiment, the fusion domain is a rhesus IgG Fc fragment or functional variant thereof.

As used herein, the Fc portion of an immunoglobulin has the meaning commonly given to the term in the field of immunology. Specifically, this term refers to an antibody fragment which does not contain the two antigen binding regions (the Fab fragments) from the antibody. The Fc portion consists of the constant region of an antibody from both heavy chains, which associate through non-covalent interactions and disulfide bonds. The Fc portion can include the hinge regions and extend through the CH2 and CH3 domains to the c-terminus of the antibody. The Fc portion can further include one or more glycosylation sites. In one embodiment, the fusion domain is a human IgG Fc. The four subclasses, IgG1, IgG2, IgG3, and IgG4, which are highly conserved, differ in their constant region, particularly in their hinges and upper CH2 domains. See. Vidarsson et al, IgG Subclasses and Allotypes:

From Structure to Effector Functions, Front Immunol. October 2014; 5:520, which is incorporated herein by reference. The Fc domain can be derived from any human IgG, including human IgG1, human IgG2, human IgG3, or human IgG4. In one embodiment, the human IgG Fc is an IgG4 Fc. In one embodiment, the human IgG Fc is SEQ ID NO: 6: AESKYGPPCPPCPAPEAAGGPSVFLFPPKPKDTLMISRTPEVTCVVVDVSQEDP EVQFNWYVDGVEVHNAKTKPREEQFNSTYRVVSVLTVLHQDWLNGKEYKCKVSN KGLPSSIEKTISKAKGQPREPQVYTLPPSQEEMTKNQVSLTCLVKGFYPSDIAVEWES NGQPENNYKTTPPVLDSDGSFFLYSRLTVDKSRWQEGNVFSCSVMHEALHNHYTQK SLSLSLG. In another embodiment, the human IgG Fc shares at least 90% identity, at least 95% identity, at least 99% identity, or at least 100% identity to SEQ ID NO: 6.

In one embodiment, the human IgG Fc is an IgG1 Fc. In one embodiment, the human IgG Fc is SEQ ID NO: 7: DKTHTCPPCPAPEAAGGPSVFLFPPKPKDTLMISRTPEVTCVVVDVSHEDPEVKFNW YVDGVEVHNAKTKPREEQYNSTYRVVSVLTVLHQDWLNGKEYKCKVSNKALPAPI EKTISKAKGQPREPQVYTLPPSRDELTKNQVS LTCLVKGFYPSDIAVEWESNGQPENNYKTTPPVLDSDGSFFLYSKLTV DKSRWQQGNVFSCSVMHEALHNHYTQKSLSLSPG. In another embodiment, the human IgG Fc shares at least 90% identity, at least 95% identity, at least 99% identity, or at least 100% identity to SEQ ID NO: 7.

In another embodiment, the fusion domain is a rhesus IgG Fc. The Fc domain can be derived from any rhesus IgG, including rhesus IgG1, rhesus IgG2, rhesus IgG3, or rhesus IgG4. In one embodiment, the rhesus IgG Fc is an IgG4 Fc. In one embodiment, the rhesus IgG Fc is SEQ ID NO: 8: PPCPPCPAPE LLGGPSVFLF PPKPKDTLMI SRTPEVTCVV VDVSQEDPEV QFNWYVDGVE VHNAQTKPRE RQFNSTYRVV SVLTVTHQDW LNGKEYTCKV SNKGLPAPIE KTISKAKGQP REPQVYILPP PQEELTKNQV SLTCLVTGFY PSDIAVEWES NGQPENTYKT TPPVLDSDGS YLLYSKLTVN KSRWQPGNIF TCSVMHEALH NHYTQKSLSV SPGK. In another embodiment, the rhesus IgG Fc shares at least 90% identity, at least 95% identity, at least 99% identity, or at least 100% identity to SEQ ID NO: 8. In one embodiment, the rhesus IgG further comprises a hinge sequence.

In another embodiment, the fusion domain is a human albumin or a functional variant thereof. In one embodiment, the human albumin is SEQ ID NO: 9: DAHKSEVAHRFKDLGEENFKALVLIAFAQYLQQCPFEDHVKLVNEVTEFAKTCVAD ESAENCDKSLHTLFGDKLCTVATLRETYGEMADCCAKQEPERNECFLQHKDDNPNL PRLVRPEVDVMCTAFHDNEETFLKKYLYEIARRHPYFY APELLFFAKRYKAAFTECC QAADKAACLLPKLDELRDEGKASSAKQRLKCASLQKFGERAFKAWAVARLSQRFP KAEFAEVSKLVTDLTKVHTECCHGDLLECADDRADLAKYICENQDSISSKLKECCEK PLLEKSHCIAEVENDEMPADLPSLAADFVESKDVCKNYAEAKDVFLGMFLYEY ARR HPDYSVVLLLRLAKTYETTLEKCCAAADPHECYAKVFDEFKPLVEEPQNLIKQNCEL FEQLGEYKFQNALLVRYTKKVPQVSTPTLVEVSRNLGKVGSKCCKHPEAKRMPCAE DYLSVVLNQLCVLHEKTPVSDRVTKCCTESLVNRRPCFSALEVDETYVPKEFNAETF TFHADICTLSEKERQIKKQTALVELVKHKPKATKEQLKAVMDDFAAFVEKCCKADD KETCFAEEGKKLVAASQAALG. In another embodiment, the human albumin shares at least 90% identity, at least 95% identity, at least 99% identity, or at least 100% identity to SEQ ID NO: 9.

The in vivo function and stability of the fusion proteins of the present disclosure may be optimized by adding small peptide linkers, e.g., to prevent potentially unwanted domain interactions or for other reasons. Further, a glycine-rich linker may provide some structural flexibility such that the PTH protein portion can interact productively with the PTH receptor on target cells such as the beta cells of the pancreas. Thus, the C-terminus of the PTH protein and the N-terminus of the fusion domain of the fusion protein are, in one embodiment, fused via a linker. In one embodiment, the linker includes 1, 1.5 or 2 repeats of a G-rich peptide linker having the sequence GGGGSGGGGSGGGGS (SEQ ID NO: 10), or similar sequence.

In one embodiment, the fusion protein comprises (a) human thrombin leader, (b) PTH (1-34), a linker, and (c) a human IgG Fc. In one embodiment, the fusion protein has the sequence of SEQ ID NO: X, or a sequence at least 90%, at least 95%, at least 98%, or at least 99% identical thereto.

SEQ ID NO: 11
MAHVRGLQLPGCLALAALCSLVHSQHVFLAPQQARSLLQRVRRSV
SEIQLMHNLGKHLNSMERVEWLRKKLQDVHNGGGGGGSGGGGSGG
GGSAESKYGPPCPPCPAPEAAGGPSVFLFPPKPKDTLMISRTPEV
TCVVVDVSQEDPEVQFNWYVDGVEVHNAKTKPREEQFNSTYRVVS
VLTVLHQDWLNGKEYKCKVSNKGLPSSIEKTISKAKGQPREPQVY
TLPPSQEEMTKNQVSLTCLVKGFYPSDIAVEWESNGQPENNYKTT
PPVLDSDGSFFLYSRLTVDKSRWQEGNVFSCSVMHEALHNHYTQK
SLSLSLG

In one embodiment, the sequence encoding the fusion protein is SEQ ID NO: 12 or a sequence at least 75%, at least 85%, at least 90%, at least 95%, at least 98%, or at least 99% identical thereto.

SEQ ID NO: 12:
ATGGCTCAcGTTCGAGGACTGCAGCTGCCTGGATGTCTGGCTCTT
GCCGCTCTGTGTAGCCTGGTGCACAGCCAGCAcGTGTTTCTGGCT
CCTCAGCAaGCCAGATCACTGCTGCAGCGCGTCAGAAGAAGCGTC
AGCGAGATCCAGCTGATGCACAACCTGGGCAAGCACCTGAACAGC
ATGGAACGCGTGGAgTGGCTGCGGAAGAAACTGCAGGACGTGCAC
AAcGGTGGCGGCGGAGGCGGAAGCGGTGGCGGAGGATCAGGCGGT
GGTGGATCTGCCGAGTCTAAGTACGGACCTCCTTGTCCTCCcTGT
CCTGCTCCcGAAGCTGCTGGCGGCCCATCCGTGTTTCTGTTCCCT
CCAAAGCCTAAGGACACCCTGATGATCAGCAGAACCCCTGAAGTG
ACCTGCGTGGTGGTGGACGTGTCCCAAGAGGATCCTGAGGTGCAG
TTCAATTGGTACGTGGACGGCGTGGAAGTGCACAACGCCAAGACC
AAGCCTAGAGAGGAACAGTTCAACAGCACCTACAGAGTGGTGTCC
GTGCTGACCGTGCTGCACCAGGATTGGCTGAACGGCAAAGAGTAC
AAGTGCAAGGTGTCCAACAAGGGCCTGCCTAGCAGCATCGAGAAA
ACCATCAGCAAGGCCAAGGGCCAGCCAAGAGAACCCCAGGTGTAC
ACACTGCCTCCAAGCCAAGAGGAAATGACCAAGAACCAGGTGTCC
CTGACCTGCCTGGTCAAGGGCTTCTACCCTTCCGATATCGCCGTC
GAGTGGGAGAGCAAcGGCCAGCCTGAGAACAACTACAAGACCACA
CCTCCTGTGCTGGACAGCGACGGCTCATTCTTCCTGTACAGCcgg
CTGACCGTGGACAAGAGCAGgTGGCAAGAGGGCAACGTGTTCAGC
TGCTCCGTGATGCACGAGGCCCTGCACAACCACTACACCCAGAAG
TCTCTGAGCCTGAGCCTGGGCTGATGA

In one embodiment, the fusion protein comprises (a) human thrombin leader, (b) PTH (1-73), a linker, and (c) a human IgG Fc. In one embodiment, the fusion protein has the sequence of SEQ ID NO: 13, or a sequence at least 90%, at least 95%, at least 98%, or at least 99% identical thereto.

SEQ ID NO: 13
MAHVRGLQLPGCLALAALCSLVHSQHVFLAPQQARSLLQRVRRSV
SEIQLMHNLGKHLNSMERVEWLRKKLQDVHNFVALGAPLAPRDAG
SQRPRKKEDNVLVESHEKSLGEADKADGGGGGGSGGGGSGGGGSA
ESKYGPPCPPCPAPEAAGGPSVFLFPPKPKDTLMISRTPEVTCVV
VDVSQEDPEVQFNWYVDGVEVHNAKTKPREEQFNSTYRVVSVLTV
LHQDWLNGKEYKCKVSNKGLPSSIEKTISKAKGQPREPQVYTLPP
SQEEMTKNQVSLTCLVKGFYPSDIAVEWESNGQPENNYKTTPPVL
DSDGSFFLYSRLTVDKSRWQEGNVFSCSVMHEALHNHYTQKSLSL
SLG**

In one embodiment, the sequence encoding the fusion protein is SEQ ID NO: 14 or a sequence at least 75%, at least 85%, at least 90%, at least 95%, at least 98%, or at least 99% identical thereto.

SEQ ID NO: 14:
ATGGCTCAcGTTCGAGGACTGCAGCTGCCTGGATGTCTGGCTCTT
GCCGCTCTGTGTAGCCTGGTGCACAGCCAGCAcGTGTTTCTGGCT
CCTCAGCAaGCCAGATCACTGCTGCAGCGCGTCAGAAGAAGCGTC
AGCGAGATCCAGCTGATGCACAACCTGGGCAAGCACCTGAACAGC
ATGGAACGCGTGGAgTGGCTGCGGAAGAAACTGCAGGACGTGCAC
AACTTCGTGGCCCTGGGAGCACCTCTGGCTCCAAGAGAcGCCGGC
AGCCAACGGCCTAGAAAGAAAGAGGACAACGTCCTGGTCGAGAGC
CACGAGAAGTCTCTGGGCGAAGCCGATAAGGCTGAcGGTGGCGGA
GGCGGAGGATCTGGTGGCGGTGGTTCTGGCGGTGGCGGATCTGCC
GAGTCTAAGTACGGACCTCCTTGTCCTCCcTGTCCTGCTCCcGAA
GCTGCTGGCGGCCCATCCGTGTTTCTGTTCCCTCCAAAGCCTAAG
GACACCCTGATGATCAGCAGAACCCCTGAAGTGACCTGCGTGGTG
GTGGACGTGTCCCAAGAGGATCCTGAGGTGCAGTTCAATTGGTAC
GTGGACGGCGTGGAAGTGCACAACGCCAAGACCAAGCCTAGAGAG
GAACAGTTCAACAGCACCTACAGAGTGGTGTCCGTGCTGACCGTG
CTGCACCAGGATTGGCTGAACGGCAAAGAGTACAAGTGCAAGGTG
TCCAACAAGGGCCTGCCTAGCAGCATCGAGAAAACCATCAGCAAG
GCCAAGGGCCAGCCAAGAGAACCCCAGGTGTACACACTGCCTCCA
AGCCAAGAGGAAATGACCAAGAACCAGGTGTCCCTGACCTGCCTG
GTCAAGGGCTTCTACCCTTCCGATATCGCCGTCGAGTGGGAGAGC
AAcGGCCAGCCTGAGAACAACTACAAGACCACACCTCCTGTGCTG
GACAGCGACGGCTCATTCTTCCTGTACAGCcggCTGACCGTGGAC
AAGAGCcggTGGCAAGAGGGCAACGTGTTCAGCTGCTCCGTGATG
CACGAGGCCCTGCACAACCACTACACCCAGAAAAGCCTGTCTCTG
AGCCTGGGCTGATGA

In one embodiment, the fusion protein comprises (a) human thrombin leader, (b) PTH (1-74), a linker, and (c) a human IgG Fc. In one embodiment, the fusion protein comprises (a) human thrombin leader, (b) PTH (1-75), a linker, and (c) a human IgG Fc. In one embodiment, the fusion protein comprises (a) human thrombin leader, (b) PTH (1-84), a linker, and (c) a human IgG Fc. In one embodiment, the fusion protein has the sequence of SEQ ID NO: 15, or a sequence at least 90%, at least 95%, at least 98%, or at least 99% identical thereto.

SEQ ID NO: 15
MAHVRGLQLPGCLALAALCSLVHSQHVFLAPQQARSLLQRVRRSV
SEIQLMHNLGKHLNSMERVEWLRKKLQDVHNFVALGAPLAPRDAG
SQRPRKKEDNVLVESHEKSLGEADKADVNVLTKAKSQGGGGGGSG
GGGSGGGGSAESKYGPPCPPCPAPEAAGGPSVFLFPPKPKDTLMI
SRTPEVTCVVVDVSQEDPEVQFNWYVDGVEVHNAKTKPREEQFNS
TYRVVSVLTVLHQDWLNGKEYKCKVSNKGLPSSIEKTISKAKGQP
REPQVYTLPPSQEEMTKNQVSLTCLVKGFYPSDIAVEWESNGQPE
NNYKTTPPVLDSDGSFFLYSRLTVDKSRWQEGNVFSCSVMHEALH
NHYTQKSLSLSLG

In one embodiment, the sequence encoding the fusion protein is SEQ ID NO: 16 or a sequence at least 75%, at least 85%, at least 90%, at least 95%, at least 98%, or at least 99% identical thereto.

SEQ ID NO: 16:
ATGGCTCAcGTTCGAGGACTGCAGCTGCCTGGATGTCTGGCTCTT
GCCGCTCTGTGTAGCCTGGTGCACAGCCAGCAcGTGTTTCTGGCT
CCTCAGCAaGCCAGATCACTGCTGCAGCGCGTCAGAAGAAGCGTC
AGCGAGATCCAGCTGATGCACAACCTGGGCAAGCACCTGAACAGC
ATGGAACGCGTGGAgTGGCTGCGGAAGAAACTGCAGGACGTGCAC
AACTTCGTGGCCCTGGGAGCACCTCTGGCTCCAAGAGAcGCCGGC
AGCCAACGGCCTAGAAAGAAAGAGGACAACGTCCTGGTCGAGAGC
CACGAGAAGTCTCTGGGCGAAGCCGATAAGGCCGACGTGAACGTG
CTGACCAAGGCCAAGTCTCAAGGTGGCGGAGGCGGAGGATCTGGT
GGTGGTGGATCTGGCGGCGGAGGTTCTGCCGAGTCTAAGTACGGA
CCTCCTTGTCCTCCcTGTCCTGCTCCcGAAGCTGCTGGCGGCCCA
TCCGTGTTTCTGTTCCCTCCAAAGCCTAAGGACACCCTGATGATC
AGCAGAACCCCTGAAGTGACCTGCGTGGTGGTCGACGTGTCCCAA
GAGGATCCTGAGGTGCAGTTCAATTGGTACGTGGACGGCGTGGAA
GTGCACAACGCCAAGACCAAGCCTAGAGAGGAACAGTTCAACAGC
ACCTACAGAGTGGTGTCTGTGCTGACCGTGCTGCACCAGGATTGG
CTGAACGGCAAAGAGTACAAGTGCAAGGTGTCCAACAAGGGCCTG
CCTAGCAGCATCGAGAAAACCATCTCTAAGGCCAAGGGCCAGCCT
CGCGAACCCCAGGTTTACACACTGCCTCCAAGCCAAGAGGAAATG
ACCAAGAACCAGGTGTCCCTGACCTGCCTGGTCAAGGGCTTCTAC
CCTTCCGATATCGCCGTCGAGTGGGAGAGCAAcGGCCAGCCTGAG
AACAACTACAAGACCACACCTCCTGTGCTGGACAGCGACGGCTCA
TTCTTCCTGTACAGCcggCTGACCGTGGACAAGAGCAGgTGGCAA
GAGGGCAACGTGTTCAGCTGCTCCGTGATGCACGAGGCCCTGCAC
AACCACTACACCCAGAAAAGCCTGTCTCTGAGCCTGGGCTGATGA

In one embodiment, the fusion protein comprises (a) human PTH leader, (b) PTH (1-34), a linker, and (c) a human IgG Fc. In one embodiment, the fusion protein has the sequence of SEQ ID NO: 17, or a sequence at least 90%, at least 95%, at least 98%, or at least 99% identical thereto.

SEQ ID NO: 17
MIPAKDMAKVMIVMLAICFLTKSDGKSVKKRSVSEIQLMHNLGKH
LNSMERVEWLRKKLQDVHNFDKTHTCPPCPAPEAAGGPSVFLFPP
KPKDTLMISRTPEVTCVVVDVSHEDPEVKFNWYVDGVEVHNAKTK
PREEQYNSTYRVVSVLTVLHQDWLNGKEYKCKVSNKALPAPIEKT
ISKAKGQPREPQVYTLPPSRDELTKNQVSLTCLVKGFYPSDIAVE
WESNGQPENNYKTTPPVLDSDGSFFLYSKLTVDKSRWQQGNVFSC
SVMHEALHNHYTQKSLSLSPG*

In one embodiment, the sequence encoding the fusion protein is SEQ ID NO: 18 or a sequence at least 75%, at least 85%, at least 90%, at least 95%, at least 98%, or at least 99% identical thereto.

SEQ ID NO: 18:
ATGATTCCCGCCAAGGACATGGCCAAAGTGATGATCGTGATGCTG
GCCATCTGCTTTCTGACCAAGAGCGACGGCAAGAGCGTGAAGAAA
AGAAGCGTGTCCGAGATCCAGCTGATGCACAACCTGGGCAAGCAC
CTGAACAGCATGGAACGCGTGGAGTGGCTGCGGAAGAAACTGCAG
GACGTGCACAACTTCGACAAGACCCACACCTGTCCTCCCTGTCCT
GCTCCCGAAGCTGCTGGCGGCCCTTCCGTGTTTCTGTTCCCTCCA
AAGCCTAAGGACACCCTGATGATCAGCAGAACCCCTGAAGTGACC
TGCGTGGTGGTGGATGTGTCCCACGAGGATCCCGAAGTGAAGTTC
AATTGGTACGTGGACGGCGTGGAAGTGCACAACGCCAAGACCAAG
CCTAGAGAGGAACAGTACAACAGCACCTACAGAGTGGTGTCCGTG
CTGACCGTGCTGCACCAGGATTGGCTGAACGGCAAAGAGTACAAG
TGCAAGGTGTCCAACAAGGCCCTGCCTGCTCCTATCGAGAAAACC
ATCAGCAAGGCCAAGGGCCAGCCTAGGGAACCCCAGGTTTACACA
CTGCCTCCAAGCAGGGACGAGCTGACAAAGAACCAGGTGTCCCTG
ACCTGCCTGGTCAAGGGCTTTTACCCCAGCGACATTGCCGTCGAG
TGGGAGTCTAACGGCCAGCCTGAGAACAACTACAAGACAACCCCT
CCTGTGCTGGACTCCGACGGCTCATTCTTCCTGTACAGCAAGCTG
ACAGTGGACAAGAGCCGGTGGCAGCAGGGCAATGTGTTCAGCTGT
AGCGTGATGCACGAGGCCCTGCACAACCACTACACCCAGAAGTCC
CTGAGCCTGTCTCCTGGCTGA

In one embodiment, the fusion protein comprises (a) human PTH leader, (b) PTH (1-73), a linker, and (c) a human IgG Fc. In one embodiment, the fusion protein has the sequence of SEQ ID NO: 19, or a sequence at least 90%, at least 95%, at least 98%, or at least 99% identical thereto.

SEQ ID NO: 19
MIPAKDMAKVMIVMLAICFLTKSDGKSVKKRSVSEIQLMHNLGKHLNSME
RVEWLRKKLQDVHNFVALGAPLAPRDAGSQRPRKKEDNVLVESHEKSLGE
ADKADKTHTCPPCPAPEAAGGPSVFLFPPKPKDTLMISRTPEVTCVVVDV
SHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSR
WQQGNVFSCSVMHEALHNHYTQKSLSLSPG*

In one embodiment, the sequence encoding the fusion protein is SEQ ID NO: 20 or a sequence at least 75%, at least 85%, at least 90%, at least 95%, at least 98%, or at least 99% identical thereto.

SEQ ID NO: 20:
ATGATTCCCGCCAAGGACATGGCCAAAGTGATGATCGTGATGCTGGCCAT
CTGCTTTCTGACCAAGAGCGACGGCAAGAGCGTGAAGAAAAGAAGCGTGT
CCGAGATCCAGCTGATGCACAACCTGGGCAAGCACCTGAACAGCATGGAA
CGCGTGGAGTGGCTGCGGAAGAAACTGCAGGACGTGCACAACTTCGTGGC
CCTGGGAGCACCTCTGGCTCCTAGGGACGCCGGCAGCCAGCGGCCTAGAA
AGAAAGAGGATAACGTCCTGGTCGAGAGCCACGAGAAGTCTCTGGGCGAA
GCCGACAAGGCCGACAAAACACACACCTGTCCTCCCTGTCCTGCTCCCGA
AGCTGCTGGCGGCCCTTCCGTGTTTCTGTTCCCTCCAAAGCCTAAGGACA
CCCTGATGATCAGCAGAACCCCTGAAGTGACCTGCGTGGTGGTGGATGTG
TCCCACGAGGATCCCGAAGTGAAGTTCAATTGGTACGTGGACGGCGTGGA
AGTCCACAACGCCAAGACCAAGCCTAGAGAGGAACAGTACAACAGCACCT
ACAGAGTGGTGTCCGTGCTGACCGTGCTGCACCAGGATTGGCTGAACGGC
AAAGAGTACAAGTGCAAGGTGTCTAACAAGGCCCTGCCTGCTCCTATCGA
GAAAACCATCAGCAAGGCCAAGGGCCAGCCTAGGGAACCCCAGGTTTACA
CACTCCCTCCAAGCAGGGACGAGCTGACAAAGAACCAGGTGTCCCTGACC
TGCCTGGTCAAGGGCTTCTACCCTTCCGATATCGCCGTCGAGTGGGAGTC
CAACGGCCAGCCAGAGAACAACTACAAGACAACCCCTCCTGTGCTGGACT
CCGACGGCTCATTCTTCCTGTACAGCAAGCTGACAGTGGACAAGAGCCGG
TGGCAGCAGGGCAATGTGTTCAGCTGTAGCGTGATGCACGAGGCCCTGCA
CAACCACTACACCCAGAAGTCCCTGAGCCTGTCTCCTGGCTGA

In one embodiment, the fusion protein comprises (a) human PTH leader, (b) PTH (1-74), a linker, and (c) a human IgG Fc. In one embodiment, the fusion protein has the sequence of SEQ ID NO: 21, or a sequence at least 90%, at least 95%, at least 98%, or at least 99% identical thereto.

SEQ ID NO: 21
MIPAKDMAKVMIVMLAICFLTKSDGKSVKKRSVSEIQLMHNLGKHLNSME
RVEWLRKKLQDVHNFVALGAPLAPRDAGSQRPRKKEDNVLVESHEKSLGE
ADKADDKTHTCPPCPAPEAAGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG*

In one embodiment, the sequence encoding the fusion protein is SEQ ID NO: 22 or a sequence at least 75%, at least 85%, at least 90%, at least 95%, at least 98%, or at least 99%

SEQ ID NO: 22:
ATGATTCCCGCCAAGGACATGGCCAAAGTGATGATCGTGATGCTGGCCAT
CTGCTTTCTGACCAAGAGCGACGGCAAGAGCGTGAAGAAAAGAAGCGTGT
CCGAGATCCAGCTGATGCACAACCTGGGCAAGCACCTGAACAGCATGGAA
CGCGTGGAGTGGCTGCGGAAGAAACTGCAGGACGTGCACAACTTCGTGGC
CCTGGGAGCACCTCTGGCTCCTAGAGACGCCGGCAGCCAGCGGCCTAGAA
AGAAAGAGGATAACGTCCTGGTCGAGAGCCACGAGAAGTCTCTGGGCGAA
GCCGACAAGGCCGACGACAAGACACACACCTGTCCTCCCTGTCCTGCTCC
CGAAGCTGCTGGCGGCCCTTCCGTGTTTCTGTTCCCTCCAAAGCCTAAGG
ACACCCTGATGATCAGCAGAACCCCTGAAGTGACCTGCGTGGTGGTGGAT
GTGTCCCACGAGGATCCCGAAGTGAAGTTCAATTGGTACGTGGACGGCGT
GGAAGTGCACAACGCCAAGACCAAGCCTAGAGAGGAACAGTACAACAGCA
CCTACAGAGTGGTGTCCGTGCTGACCGTGCTGCACCAGGATTGGCTGAAC
GGCAAAGAGTACAAGTGCAAGGTGTCCAACAAGGCCCTGCCTGCTCCTAT
CGAGAAAACCATCAGCAAGGCCAAGGGCCAGCCTAGGGAACCCCAGGTTT
ACACACTGCCTCCAAGCAGGGACGAGCTGACAAAGAACCAGGTGTCCCTG
ACCTGCCTGGTCAAGGGCTTCTACCCTTCCGATATCGCCGTCGAGTGGGA
GTCCAACGGCCAGCCAGAGAACAACTACAAGACAACCCCTCCTGTGCTGG
ACTCCGACGGCTCATTCTTCCTGTACAGCAAGCTGACAGTGGACAAGAGC
CGGTGGCAGCAGGGCAATGTGTTCAGCTGTAGCGTGATGCACGAGGCCCT
GCACAACCACTACACCCAGAAGTCCCTGAGCCTGTCTCCTGGCTGA

In one embodiment, the fusion protein comprises (a) human PTH leader (including propeptide), (b) PTH (1-75), a linker, and (c) a human IgG Fc. In one embodiment, the fusion protein has the sequence of SEQ ID NO: 23, or a sequence at least 90%, at least 95%, at least 98%, or at least 99% identical thereto.

SEQ ID NO: 23
MIPAKDMAKVMIVMLAICFLTKSDGKSVKKRSVSEIQLMHNLGKHLNSME
RVEWLRKKLQDVHNFVALGAPLAPRDAGSQRPRKKEDNVLVESHEKSLGE
ADKADVDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDK
SRWQQGNVFSCSVMHEALHNHYTQKSLSLSPG

In one embodiment, the sequence encoding the fusion protein is SEQ ID NO: 24 or a sequence at least 75%, at least 85%, at least 90%, at least 95%, at least 98%, or at least 99% identical thereto.

SEQ ID NO: 24:
ATGATTCCCGCCAAGGACATGGCCAAAGTGATGATCGTGATGCTGGCCAT
CTGCTTTCTGACCAAGAGCGACGGCAAGAGCGTGAAGAAAAGAAGCGTGT
CCGAGATCCAGCTGATGCACAACCTGGGCAAGCACCTGAACAGCATGGAA
CGCGTGGAGTGGCTGCGGAAGAAACTGCAGGACGTGCACAACTTCGTGGC
CCTGGGAGCACCTCTGGCTCCTAGAGACGCCGGCAGCCAGCGGCCTAGAA
AGAAAGAGGATAACGTCCTGGTCGAGAGCCACGAGAAGTCTCTGGGCGAA
GCCGACAAGGCCGACGTGGACAAGACACACACCTGTCCTCCCTGTCCTGC
TCCCGAAGCTGCTGGCGGCCCTTCCGTGTTTCTGTTCCCTCCAAAGCCTA
AGGACACCCTGATGATCAGCAGAACCCCTGAAGTGACCTGCGTGGTGGTG
GATGTGTCCCACGAGGATCCCGAAGTGAAGTTCAATTGGTACGTGGACGG
CGTGGAAGTGCACAACGCCAAGACCAAGCCTAGAGAGGAACAGTACAACA
GCACCTACAGAGTGGTGTCCGTGCTGACCGTGCTGCACCAGGATTGGCTG
AACGGCAAAGAGTACAAGTGCAAGGTGTCCAACAAGGCCCTGCCTGCTCC
TATCGAGAAAACCATCAGCAAGGCCAAGGGCCAGCCTAGGGAACCCCAGG
TTTACACACTGCCTCCAAGCAGGGACGAGCTGACAAAGAACCAGGTGTCC
CTGACCTGCCTGGTCAAGGGCTTCTACCCTTCCGATATCGCCGTCGAGTG
GGAGTCCAACGGCCAGCCAGAGAACAACTACAAGACAACCCCTCCTGTGC
TGGACTCCGACGGCTCATTCTTCCTGTACAGCAAGCTGACAGTGGACAAG
AGCCGGTGGCAGCAGGGCAATGTGTTCAGCTGTAGCGTGATGCACGAGGC
CCTGCACAACCACTACACCCAGAAGTCCCTGAGCCTGTCTCCTGGCTGA

In one embodiment, the fusion protein comprises (a) human PTH leader, (b) PTH (1-84), a linker, and (c) a human IgG Fc.

In one embodiment, the fusion protein comprises (a) human IL2 leader (including PTH or thrombin propeptide), (b) PTH (1-34), a linker, and (c) a human IgG Fc. In one embodiment, the fusion protein comprises (a) human IL2 leader (including PTH or thrombin propeptide), (b) PTH (1-73), a linker, and (c) a human IgG Fc. In one embodiment, the fusion protein comprises (a) human IL2 leader (including PTH or thrombin propeptide), (b) PTH (1-74), a linker, and (c) a human IgG Fc. In one embodiment, the fusion protein comprises (a) human IL2 leader (including PTH or thrombin propeptide), (b) PTH (1-75), a linker, and (c) a human IgG Fc. In one embodiment, the fusion protein comprises (a) human IL2 leader (including PTH or thrombin propeptide), (b) PTH (1-84), a linker, and (c) a human IgG Fc.

In one embodiment, the fusion protein comprises (a) human thrombin leader, (b) PTH (1-34), a linker, and (c) a human Albumin. In one embodiment, the fusion protein has the sequence of SEQ ID NO: 25, or a sequence at least 90%, at least 95%, at least 98%, or at least 99% identical thereto.

SEQ ID NO: 25
MAHVRGLQLPGCLALAALCSLVHSQHVFLAPQQARSLLQRVRRSVSEIQL
MHNLGKHLNSMERVEWLRKKLQDVHNGGGGGGSGGGGSGGGGSDAHKSEV
AHRFKDLGEENFKALVLIAFAQYLQQCPFEDHVKLVNEVTEFAKTCVADE
SAENCDKSLHTLFGDKLCTVATLRETYGEMADCCAKQEPERNECFLQHKD
DNPNLPRLVRPEVDVMCTAFHDNEETFLKKYLYEIARRHPYFYAPELLFF
AKRYKAAFTECCQAADKAACLLPKLDELRDEGKASSAKQRLKCASLQKFG
ERAFKAWAVARLSQRFPKAEFAEVSKLVTDLTKVHTECCHGDLLECADDR
ADLAKYICENQDSISSKLKECCEKPLLEKSHCIAEVENDEMPADLPSLAA
DFVESKDVCKNYAEAKDVFLGMFLYEYARRHPDYSVVLLLRLAKTYETTL
EKCCAAADPHECYAKVFDEFKPLVEEPQNLIKQNCELFEQLGEYKFQNAL
LVRYTKKVPQVSTPTLVEVSRNLGKVGSKCCKHPEAKRMPCAEDYLSVVL
NQLCVLHEKTPVSDRVTKCCTESLVNRRPCFSALEVDETYVPKEFNAETF
TFHADICTLSEKERQIKKQTALVELVKHKPKATKEQLKAVMDDFAAFVEK
CCKADDKETCFAEEGKKLVAASQAALG

In one embodiment, the sequence encoding the fusion protein is SEQ ID NO: 26 or a sequence at least 75%, at least 85%, at least 90%, at least 95%, at least 98%, or at least 99% identical thereto.

SEQ ID NO: 26:
ATGGCTCAcGTTCGAGGACTGCAGCTGCCTGGATGTCTGGCTCTTGCCGC
TCTGTGTAGCCTGGTGCACAGCCAGCAcGTGTTTCTGGCTCCTCAGCAaG
CCAGATCACTGCTGCAGCGCGTCAGAAGAAGCGTCAGCGAGATCCAGCTG
ATGCACAACCTGGGCAAGCACCTGAACAGCATGGAACGCGTGGAgTGGCT
GCGGAAGAAACTGCAGGACGTGCACAAcGGTGGCGGCGGAGGCGGAAGCG
GTGGCGGAGGATCAGGCGGTGGTGGATCTGAcGCCCACAAGTCTGAAGTG
GCCCACCGGTTCAAGGACCTGGGCGAAGAGAATTTCAAGGCCCTGGTGCT
GATCGCCTTCGCTCAGTACCTGCAGCAgTGCCCCTTCGAGGACCACGTGA
AGCTGGTCAACGAAGTGACCGAGTTCGCCAAGACCTGCGTGGCCGAcGAG
AGCGCCGAGAACTGTGATAAGAGCCTGCACACCCTGTTCGGCGACAAGCT
GTGTACAGTGGCCACACTGAGAGAAACCTACGGCGAGATGGCCGACTGCT
GCGCCAAGCAAGAGCCCGAGAGAAACGAGTGCTTCCTGCAGCACAAGGAC
GACAACCCCAACCTGCCTAGACTCGTGCGGCCTGAAGTGGACGTGATGTG
CACCGCCTTCCACGACAACGAGGAAACCTTCCTGAAGAAGTACCTGTACG
AGATCGCCAGACGGCACCCCTACTTTTACGCCCCTGAGCTGCTGTTCTTC
GCCAAGCGGTATAAGGCCGCCTTCACCGAGTGTTGTCAGGCCGCTGATAA
GGCTGCCTGCCTGCTGCCTAAGCTGGACGAGCTTAGAGAcGAGGGCAAAG
CCAGCTCCGCCAAGCAGAGACTGAAGTGTGCCAGCCTGCAGAAGTTCGGC
GAGAGAGCCTTTAAGGCCTGGGCCGTTGCTAGACTGAGCCAGAGATTTCC
CAAGGCCGAGTTTGCCGAGGTGTCCAAGCTCGTGACCGACCTGACAAAGG
TGCACACCGAgTGCTGCCACGGCGACCTGCTGGAATGCGCCGAcGATAGA
GCCGACCTGGCCAAGTACATCTGCGAGAACCAGGACAGCATCAGCAGCAA
GCTGAAAGAGTGCTGCGAGAAGCCTCTGCTGGAAAAGAGCCACTGTATCG
CCGAGGTGGAAAACGACGAGATGCCCGCCGATCTGCCTTCTCTGGCCGCC
GATTTTGTGGAAAGCAAGGACGTGTGCAAGAACTACGCCGAGGCCAAGGA
cGTGTTCCTGGGCATGTTTCTGTAcGAGTACGCCCGCAGACACCCCGACT
ACTCTGTTGTGCTGCTGCTGAGACTGGCCAAAACCTACGAGACAACCCTG
GAAAAGTGCTGTGCCGCCGCTGATCCTCACGAGTGTTACGCCAAGGTGTT
CGACGAGTTCAAGCCACTGGTGGAAGAACCCCAGAACCTGATCAAGCAGA
ACTGCGAGCTGTTCGAGCAGCTGGGCGAGTACAAGTTCCAGAACGCCCTG
CTCGTGCGGTACACCAAGAAGGTGCCCCAGGTTTCCACACCTACACTGGT
TGAGGTGTCCCGGAATCTGGGCAAAGTGGGCAGCAAGTGCTGCAAGCACC
CTGAGGCCAAGAGAATGCCTTGCGCCGAGGATTACCTGAGCGTGGTGCTG
AATCAGCTGTGCGTGCTGCACGAGAAAACCCCTGTGTCCGACAGAGTGAC
CAAGTGCTGTACCGAGAGCCTGGTCAACAGACGGCCTTGCTTTAGCGCCC
TCGAGGTGGACGAGACATACGTGCCCAAAGAGTTCAACGCCGAGACATTC
ACCTTCCACGCCGACATCTGTACCCTGAGCGAGAAAGAGCGGCAGATCAA
GAAACAGACTGCCCTGGTGGAACTGGTCAAGCACAAGCCCAAGGCCACCA
AAGAACAGCTGAAGGCCGTGATGGACGACTTCGCCGCCTTCGTGGAAAAA
TGCTGCAAGGCCGACGACAAAGAGACATGCTTCGCCGAAGAGGGCAAGAA
ACTGGTGGCCGCTTCTCAGGCTGCCCTGGGATGATAA

In one embodiment, the fusion protein comprises (a) human thrombin leader, (b) PTH (1-73), a linker, and (c) a human Albumin. In one embodiment, the fusion protein has the sequence of SEQ ID NO: 27, or a sequence at least 90%, at least 95%, at least 98%, or at least 99% identical thereto.

SEQ ID NO: 27
MAHVRGLQLPGCLALAALCSLVHSQHVFLAPQQARSLLQRVRRSVSEIQL
MHNLGKHLNSMERVEWLRKKLQDVHNGGGGGGSGGGGSGGGGSDAHKSEV
AHRFKDLGEENFKALVLIAFAQYLQQCPFEDHVKLVNEVTEFAKTCVADE
SAENCDKSLHTLFGDKLCTVATLRETYGEMADCCAKQEPERNECFLQHKD
DNPNLPRLVRPEVDVMCTAFHDNEETFLKKYLYEIARRHPYFYAPELLFF
AKRYKAAFTECCQAADKAACLLPKLDELRDEGKASSAKQRLKCASLQKFG
ERAFKAWAVARLSQRFPKAEFAEVSKLVTDLTKVHTECCHGDLLECADDR
ADLAKYICENQDSISSKLKECCEKPLLEKSHCIAEVENDEMPADLPSLAA
DFVESKDVCKNYAEAKDVFLGMFLYEYARRHPDYSVVLLLRLAKTYETTL
EKCCAAADPHECYAKVFDEFKPLVEEPQNLIKQNCELFEQLGEYKFQNAL
LVRYTKKVPQVSTPTLVEVSRNLGKVGSKCCKHPEAKRMPCAEDYLSVVL
NQLCVLHEKTPVSDRVTKCCTESLVNRRPCFSALEVDETYVPKEFNAETF
TFHADICTLSEKERQIKKQTALVELVKHKPKATKEQLKAVMDDFAAFVEK
CCKADDKETCFAEEGKKLVAASQAALG

In one embodiment, the sequence encoding the fusion protein is SEQ ID NO: 28 or a sequence at least 75%, at least 85%, at least 90%, at least 95%, at least 98%, or at least 99% identical thereto.

SEQ ID NO: 28:
ATGGCTCAcGTTCGAGGACTGCAGCTGCCTGGATGTCTGGCTCTTGCCGC
TCTGTGTAGCCTGGTGCACAGCCAGCAcGTGTTTCTGGCTCCTCAGCAaG
CCAGATCACTGCTGCAGCGCGTCAGAAGAAGCGTCAGCGAGATCCAGCTG
ATGCACAACCTGGGCAAGCACCTGAACAGCATGGAACGCGTGGAgTGGCT
GCGGAAGAAACTGCAGGACGTGCACAAcGGTGGCGGCGGAGGCGGAAGCG
GTGGCGGAGGATCAGGCGGTGGTGGATCTGAcGCCCACAAGTCTGAAGTG
GCCCACCGGTTCAAGGACCTGGGCGAAGAGAATTTCAAGGCCCTGGTGCT
GATCGCCTTCGCTCAGTACCTGCAGCAgTGCCCCTTCGAGGACCACGTGA
AGCTGGTCAACGAAGTGACCGAGTTCGCCAAGACCTGCGTGGCCGAcGAG
AGCGCCGAGAACTGTGATAAGAGCCTGCACACCCTGTTCGGCGACAAGCT
GTGTACAGTGGCCACACTGAGAGAAACCTACGGCGAGATGGCCGACTGCT
GCGCCAAGCAAGAGCCCGAGAGAAACGAGTGCTTCCTGCAGCACAAGGAC
GACAACCCCAACCTGCCTAGACTCGTGCGGCCTGAAGTGGACGTGATGTG
CACCGCCTTCCACGACAACGAGGAAACCTTCCTGAAGAAGTACCTGTACG
AGATCGCCAGACGGCACCCCTACTTTTACGCCCCTGAGCTGCTGTTCTTC
GCCAAGCGGTATAAGGCCGCCTTCACCGAGTGTTGTCAGGCCGCTGATAA
GGCTGCCTGCCTGCTGCCTAAGCTGGACGAGCTTAGAGAcGAGGGCAAAG
CCAGCTCCGCCAAGCAGAGACTGAAGTGTGCCAGCCTGCAGAAGTTCGGC
GAGAGAGCCTTTAAGGCCTGGGCCGTTGCTAGACTGAGCCAGAGATTTCC
CAAGGCCGAGTTTGCCGAGGTGTCCAAGCTCGTGACCGACCTGACAAAGG
TGCACACCGAgTGCTGCCACGGCGACCTGCTGGAATGCGCCGAcGATAGA
GCCGACCTGGCCAAGTACATCTGCGAGAACCAGGACAGCATCAGCAGCAA
GCTGAAAGAGTGCTGCGAGAAGCCTCTGCTGGAAAAGAGCCACTGTATCG
CCGAGGTGGAAAACGACGAGATGCCCGCCGATCTGCCTTCTCTGGCCGCC
GATTTTGTGGAAAGCAAGGACGTGTGCAAGAACTACGCCGAGGCCAAGGA
cGTGTTCCTGGGCATGTTTCTGTAcGAGTACGCCCGCAGACACCCCGACT
ACTCTGTTGTGCTGCTGCTGAGACTGGCCAAAACCTACGAGACAACCCTG
GAAAAGTGCTGTGCCGCCGCTGATCCTCACGAGTGTTACGCCAAGGTGTT
CGACGAGTTCAAGCCACTGGTGGAAGAACCCCAGAACCTGATCAAGCAGA
ACTGCGAGCTGTTCGAGCAGCTGGGCGAGTACAAGTTCCAGAACGCCCTG
CTCGTGCGGTACACCAAGAAGGTGCCCCAGGTTTCCACACCTACACTGGT
TGAGGTGTCCCGGAATCTGGGCAAAGTGGGCAGCAAGTGCTGCAAGCACC
CTGAGGCCAAGAGAATGCCTTGCGCCGAGGATTACCTGAGCGTGGTGCTG
AATCAGCTGTGCGTGCTGCACGAGAAAACCCCTGTGTCCGACAGAGTGAC
CAAGTGCTGTACCGAGAGCCTGGTCAACAGACGGCCTTGCTTTAGCGCCC
TCGAGGTGGACGAGACATACGTGCCCAAAGAGTTCAACGCCGAGACATTC
ACCTTCCACGCCGACATCTGTACCCTGAGCGAGAAAGAGCGGCAGATCAA
GAAACAGACTGCCCTGGTGGAACTGGTCAAGCACAAGCCCAAGGCCACCA
AAGAACAGCTGAAGGCCGTGATGGACGACTTCGCCGCCTTCGTGGAAAAA
TGCTGCAAGGCCGACGACAAAGAGACATGCTTCGCCGAAGAGGGCAAGAA
ACTGGTGGCCGCTTCTCAGGCTGCCCTGGGATGATAA

In one embodiment, the fusion protein comprises (a) human thrombin leader, (b) PTH (1-74), a linker, and (c) a human Albumin. In one embodiment, the fusion protein comprises (a) human thrombin leader (b) PTH (1-75), a linker, and (c) a human Albumin. In one embodiment, the fusion protein comprises (a) human thrombin leader, (b) PTH (1-84), a linker, and (c) a human Albumin. In one embodiment, the fusion protein has the sequence of SEQ ID NO: 29, or a sequence at least 90%, at least 95%, at least 98%, or at least 99% identical thereto.

SEQ ID NO: 29
MAHVRGLQLPGCLALAALCSLVHSQHVFLAPQQARSLLQRVRRSVSEIQL
MHNLGKHLNSMERVEWLRKKLQDVHNFVALGAPLAPRDAGSQRPRKKEDN
VLVESHEKSLGEADKADVNVLTKAKSQGGGGGGSGGGGSGGGGSDAHKSE
VAHRFKDLGEENFKALVLIAFAQYLQQCPFEDHVKLVNEVTEFAKTCVAD
ESAENCDKSLHTLFGDKLCTVATLRETYGEMADCCAKQEPERNECFLQHK
DDNPNLPRLVRPEVDVMCTAFHDNEETFLKKYLYEIARRHPYFYAPELLF
FAKRYKAAFTECCQAADKAACLLPKLDELRDEGKASSAKQRLKCASLQKF
GERAFKAWAVARLSQRFPKAEFAEVSKLVTDLTKVHTECCHGDLLECADD
RADLAKYICENQDSISSKLKECCEKPLLEKSHCIAEVENDEMPADLPSLA
ADFVESKDVCKNYAEAKDVFLGMFLYEYARRHPDYSVVLLLRLAKTYETT
LEKCCAAADPHECYAKVFDEFKPLVEEPQNLIKQNCELFEQLGEYKFQNA
LLVRYTKKVPQVSTPTLVEVSRNLGKVGSKCCKHPEAKRMPCAEDYLSVV
LNQLCVLHEKTPVSDRVTKCCTESLVNRRPCFSALEVDETYVPKEFNAET
FTFHADICTLSEKERQIKKQTALVELVKHKPKATKEQLKAVMDDFAAFVE
KCCKADDKETCFAEEGKKLVAASQAALG

In one embodiment, the sequence encoding the fusion protein is SEQ ID NO: 30 or a sequence at least 75%, at least 85%, at least 90%, at least 95%, at least 98%, or at least 99% identical thereto.

SEQ ID NO: 30:
ATGGCTCAcGTTCGAGGACTGCAGCTGCCTGGATGTCTGGCTCTTGCCGC
TCTGTGTAGCCTGGTGCACAGCCAGCAcGTGTTTCTGGCTCCTCAGCAaG
CCAGATCACTGCTGCAGCGCGTCAGAAGAAGCGTCAGCGAGATCCAGCTG
ATGCACAACCTGGGCAAGCACCTGAACAGCATGGAACGCGTGGAgTGGCT
GCGGAAGAAACTGCAGGACGTGCACAACTTCGTGGCCCTGGGAGCACCTC
TGGCTCCAAGAGAcGCCGGCAGCCAACGGCCTAGAAAGAAAGAGGACAAC
GTCCTGGTCGAGAGCCACGAGAAGTCTCTGGGCGAAGCCGATAAGGCCGA
CGTGAACGTGCTGACCAAGGCCAAGTCTCAAGGTGGCGGAGGCGGAGGAT
CTGGTGGTGGTGGATCTGGCGGCGGAGGTTCTGAcGCCCACAAATCTGAA
GTGGCCCACCGGTTCAAGGACCTGGGAGAAGAGAACTTCAAGGCCCTGGT
GCTGATCGCCTTCGCTCAGTACCTGCAGCAgTGCCCCTTCGAGGACCACG
TGAAGCTGGTCAACGAAGTGACCGAGTTCGCCAAGACCTGCGTGGCCGAc
GAGAGCGCCGAGAACTGTGATAAGAGCCTGCACACCCTGTTCGGCGACAA
GCTGTGTACAGTGGCCACACTGAGAGAAACCTACGGCGAGATGGCCGACT
GCTGCGCCAAGCAAGAGCCCGAGAGAAACGAGTGCTTCCTGCAGCACAAG
GACGACAACCCCAACCTGCCTAGACTCGTGCGGCCTGAAGTGGACGTGAT
GTGCACCGCCTTCCACGACAACGAGGAAACCTTCCTGAAGAAGTACCTGT
ACGAGATCGCCAGACGGCACCCCTACTTTTACGCCCCTGAGCTGCTGTTC
TTCGCCAAGCGGTATAAGGCCGCCTTCACCGAGTGTTGTCAGGCCGCTGA
TAAGGCTGCCTGCCTGCTGCCTAAGCTGGACGAGCTTAGAGAcGAGGGCA
AAGCCAGCTCCGCCAAGCAGAGACTGAAGTGTGCCAGCCTGCAGAAGTTC
GGCGAGAGAGCCTTTAAGGCCTGGGCCGTTGCTAGACTGAGCCAGAGATT
TCCCAAGGCCGAGTTTGCCGAGGTGTCCAAGCTCGTGACCGACCTGACAA
AGGTGCACACCGAgTGCTGCCACGGCGACCTGCTGGAATGCGCCGAcGAT
AGAGCCGACCTGGCCAAGTACATCTGCGAGAACCAGGACAGCATCAGCAG
CAAGCTGAAAGAGTGCTGCGAGAAGCCTCTGCTGGAAAAGAGCCACTGTA
TCGCCGAGGTGGAAAACGACGAGATGCCCGCCGATCTGCCTTCTCTGGCC
GCCGATTTTGTGGAAAGCAAGGACGTGTGCAAGAACTACGCCGAGGCCAA
GGAcGTGTTCCTGGGCATGTTTCTGTAcGAGTACGCCCGCAGACACCCCG
ACTACTCTGTTGTGCTGCTGCTGAGACTGGCCAAAACCTACGAGACAACC
CTGGAAAAGTGCTGTGCCGCCGCTGATCCTCACGAGTGTTACGCCAAGGT
GTTCGACGAGTTCAAGCCACTGGTGGAAGAACCCCAGAACCTGATCAAGC
AGAACTGCGAGCTGTTCGAGCAGCTGGGCGAGTACAAGTTCCAGAACGCC
CTGCTCGTGCGGTACACCAAGAAGGTGCCCCAGGTTTCCACACCTACACT
GGTTGAGGTGTCCCGGAATCTGGGCAAAGTGGGCAGCAAGTGCTGCAAGC
ACCCTGAGGCCAAGAGAATGCCTTGCGCCGAGGATTACCTGAGCGTGGTG
CTGAATCAGCTGTGCGTGCTGCACGAGAAAACCCCTGTGTCCGACAGAGT
GACCAAGTGCTGTACCGAGAGCCTGGTCAACAGACGGCCTTGCTTTAGCG
CCCTCGAGGTGGACGAGACATACGTGCCCAAAGAGTTCAACGCCGAGACA
TTCACCTTCCACGCCGACATCTGTACCCTGAGCGAGAAAGAGCGGCAGAT
CAAGAAACAGACTGCCCTGGTGGAACTGGTCAAGCACAAGCCCAAGGCCA
CCAAAGAACAGCTGAAGGCCGTGATGGACGACTTCGCCGCCTTCGTGGAA
AAATGCTGCAAGGCCGAcGACAAAGAGACATGCTTCGCCGAAGAGGGCAA
GAAACTGGTGGCCGCTTCTCAGGCTGCCCTGGGATGATAA

In one embodiment, the fusion protein comprises (a) human PTH leader (including propeptide), (b) PTH (1-34), a linker, and (c) a human Albumin. In one embodiment, the fusion protein comprises (a) human PTH leader (including propeptide), (b) PTH (1-73), a linker, and (c) a human Albumin. In one embodiment, the fusion protein comprises (a) human PTH leader (including propeptide), (b) PTH (1-74), a linker, and (c) a human Albumin. In one embodiment, the fusion protein comprises (a) human PTH leader (including propeptide), (b) PTH (1-75), a linker, and (c) a human Albumin. In one embodiment, the fusion protein comprises (a) human PTH leader (including propeptide), (b) PTH (1-84), a linker, and (c) a human Albumin.

In one embodiment, the fusion protein comprises (a) human IL2 leader (including PTH or thrombin propeptide), (b) PTH (1-34), a linker, and (c) a human Albumin. In one embodiment, the fusion protein comprises (a) human IL2 leader (including PTH or thrombin propeptide), (b) PTH (1-73), a linker, and (c) a human Albumin. In one embodiment, the fusion protein comprises (a) human IL2 leader (including PTH or thrombin propeptide), (b) PTH (1-74), a linker, and (c) a human Albumin. In one embodiment, the fusion protein comprises (a) human IL2 leader (including PTH or thrombin propeptide), (b) PTH (1-75), a linker, and (c) a human Albumin. In one embodiment, the fusion protein comprises (a) human IL2 leader (including PTH or thrombin propeptide), (b) PTH (1-84), a linker, and (c) a human Albumin.

When a variant or fragment of the leader sequence, PTH protein, or fusion domain is desired, the coding sequences for these peptides may be generated using site-directed mutagenesis of the wild-type nucleic acid sequence. Alternatively, or additionally, web-based or commercially available computer programs, as well as service-based companies may be used to back translate the amino acids sequences to nucleic acid coding sequences, including both RNA and/or cDNA. See, e.g., backtranseq by EMBOSS, ebi.ac.uk/Tools/st/; Gene Infinity (geneinfinity.org/sms-/sms_backtranslation.html); ExPasy (expasy.org/tools/). In one embodiment, the RNA and/or cDNA coding sequences are designed for optimal expression in the subject species for which administration is ultimately intended, e.g., a human.

The coding sequences may be designed for optimal expression using codon optimization. Codon-optimized coding regions can be designed by various different methods. This optimization may be performed using methods which are available on-line, published methods, or a company which provides codon optimizing services. One codon optimizing method is described, e.g., in International Patent Application Pub. No. WO 2015/012924, which is incorporated by reference herein. Briefly, the nucleic acid sequence encoding the product is modified with synonymous codon sequences. Suitably, the entire length of the open reading frame (ORF) for the product is modified. However, in some embodiments, only a fragment of the ORF may be altered. By using one of these methods, one can apply the frequencies to any given polypeptide sequence, and produce a nucleic acid fragment of a codon-optimized coding region which encodes the polypeptide.

In addition to the leader sequences, PTH proteins, fusion domains, and fusion proteins provided herein, nucleic acid sequences encoding these polypeptides are provided. In one embodiment, a nucleic acid sequence is provided which encodes for the PTH peptides described herein. In some embodiments, this may include any nucleic acid sequence which encodes the PTH sequence of SEQ ID NO: 1. In another embodiment, this includes any nucleic acid which includes the PTH sequence of SEQ ID NO: 2.

In one embodiment, a nucleic acid sequence is provided which encodes for the PTH fusion protein described herein. In another embodiment, this includes any nucleic acid sequence which encodes the PTH fusion protein of SEQ ID NO: 1, 11, 13, 15, 17, 19, 21, 23, 25, 27, 29, 39, 42, 44, 46, or 48.

### Expression Cassettes

Provided herein, in another aspect, is an expression cassette comprising a nucleic acid encoding a PTH fusion protein as described herein. As used herein, an “expression cassette” refers to a nucleic acid molecule which comprises a biologically useful nucleic acid sequence (e.g., a gene cDNA encoding a protein, enzyme or other useful gene product, mRNA, etc.) and regulatory sequences operably linked thereto which direct or modulate transcription, translation, and/or expression of the nucleic acid sequence and its gene product. As used herein, “operably linked” sequences include both regulatory sequences (also referred to as elements) that are contiguous or non-contiguous with the nucleic acid sequence and regulatory sequences that act in trans or cis nucleic acid sequence. Such regulatory sequences typically include, e.g., one or more of a promoter, an enhancer, a transcription factor, transcription terminator, an intron, sequences that enhance translation efficiency (i.e., a Kozak consensus sequence), efficient RNA processing signals such as slicing and a polyadenylation sequence, sequences that stabilize cytoplasmic mRNA, for example Woodchuck Hepatitis Virus (WHP) posttranslational Regulatory Element (WPRE), and a TATA signal. The expression cassette may contain regulatory sequences upstream (5′ to) of the gene sequence, e.g., one or more of a promoter, an enhancer, an intron, etc., and one or more of an enhancer, or regulatory sequences downstream (3′ to) a gene sequence, e.g., 3′ untranslated region (3′ UTR) comprising a polyadenylation site, among other elements. In certain embodiments, the regulatory sequences are operably linked to the nucleic acid sequence of a gene product, wherein the regulatory sequences are separated from nucleic acid sequence of a gene product by intervening nucleic acid sequences, i.e., 5′-untranslated regions (5′UTR). In certain embodiments, the expression cassette comprises nucleic acid sequence of one or more of gene products. In some embodiments, the expression cassette can be a monocistronic or a bicistronic expression cassette. In other embodiments, the term “transgene” refers to one or more DNA sequences from an exogenous source which are inserted into a target cell.

In one embodiment, the expression cassette refers to a nucleic acid molecule which comprises the PTH construct coding sequences (e.g., coding sequences for the PTH fusion protein), promoter, and may include other regulatory sequences therefor, which cassette may be engineered into a genetic element and/or packaged into the capsid of a viral vector (e.g., a viral particle). Typically, such an expression cassette for generating a viral vector contains the PTH construct sequences described herein flanked by packaging signals of the viral genome (and is termed a “vector genome”) and other expression control sequences such as those described herein. Any of the expression control sequences can be optimized for a specific species using techniques known in the art including, e.g., codon optimization, as described herein.

In certain embodiments, the expression cassette includes a constitutive promoter. In another embodiment, a CB7 promoter is used. CB7 is a chicken β-actin promoter with cytomegalovirus enhancer elements. In some embodiments, the CB7 promoter has the nucleic acid sequence of SEQ ID NO: 31:

ctagtcgacattgattattgactagttattaatagtaatcaattacgggg
tcattagttcatagcccatatatggagttccgcgttacataacttacggt
aaatggcccgcctggctgaccgcccaacgacccccgcccattgacgtcaa
taatgacgtatgttcccatagtaacgccaatagggactttccattgacgt
caatgggtggagtatttacggtaaactgcccacttggcagtacatcaagt
gtatcatatgccaagtacgccccctattgacgtcaatgacggtaaatggc
ccgcctggcattatgcccagtacatgaccttatgggactttcctacttgg
cagtacatctacgtattagtcatcgctattaccatggtcgaggtgagccc
cacgttctgcttcactctccccatctcccccccctccccacccccaattt
tgtatttatttattttttaattattttgtgcagcgatgggggcggggggg
gggggggggcgcgcgccaggcggggggggggggcgaggggcggggcgggg
cgaggcggagaggtgcggcggcagccaatcagagcggcgcgctccgaaag
tttccttttatggcgaggcggcggcggcggcggccctataaaaagcgaag
cgcgcggcgggcg

In one embodiment, the promoter is a CMV promoter. In some embodiments, the CMV promoter has the nucleic acid sequence of SEQ ID NO: 32:

cgttacataacttacggtaaatggcccgcctggctgaccgcccaacgacc
cccgcccattgacgtcaataatgacgtatgttcccatagtaacgccaata
gggactttccattgacgtcaatgggtggagtatttacggtaaactgccca
cttggcagtacatcaagtgtatcatatgccaagtacgccccctattgacg
tcaatgacggtaaatggcccgcctggcattatgcccagtacatgacctta
tgggactttcctacttggcagtacatctacgtattagtcatcgctattac
catggtgatgcggttttggcagtacatcaatgggcgtggatagcggtttg
actcacggggatttccaagtctccaccccattgacgtcaatgggagtttg
ttttggcaccaaaatcaacgggactttccaaaatgtcgtaacaactccgc
cccattgacgcaaatgggcggtaggcgtgtacggtgggaggtctatataa
gcagagctcgtttagtgaaccgtcagatc

In another embodiment, a tissue specific promoter is used. Alternatively, other liver-specific promoters may be used such as those listed in the Liver Specific Gene Promoter

Database, Cold Spring Harbor. (rulai.schl.edu/LSPD), and including, but not limited to, alpha 1 anti-trypsin (A1AT); human albumin (Miyatake et al., J. Virol., 71:5124 32 (1997)), humAlb: hepatitis B virus core promoter (Sandig et al., Gene Ther., 3:1002 9 (1996)); a TTR minimal enhancer/promoter, alpha-antitrypsin promoter, liver-specific promoter (LSP) (Wu et al. Mol Ther. 16:280-289 (2008)), TBG liver specific promoter. Other promoters, such as viral promoters, constitutive promoters, regulatable promoters (see, e.g., WO 2011/126808 and WO 2013/04943), or a promoter responsive to physiologic cues may be used may be utilized in the vectors described herein.

In one embodiment, the promoter is comprised in an inducible gene expression system. The inducible gene regulation/expression system contains at least the following components: a promoter operably linked to transgene encoding the PTH fusion protein described herein (also referred to as the regulatable promoter), an activation domain, DNA binding domain, and zinc finger homeodomain binding site(s). In other embodiments, additional components may be included in the expression system, as further described herein.

The system comprises the promoter upstream of the coding sequence for the PTH fusion protein. Promoters described herein, such as CMV and CB7 promoters may be used. In one embodiment, the promoter is a CMV promoter, such as that shown in SEQ ID NO: 32.In another embodiment, the promoter is the ubiquitous, inducible promoter Z12I which comprises 12 repeated copies of the binding site for ZFHD1 and the IL2 minimal promoter. See, e.g., Chen et al, Hum Gene Ther Methods. 2013 Aug; 24(4): 270-278, which is incorporated herein.

The expression system comprises an activation domain, which is preferably located upstream of the DNA binding domain. In one embodiment, the activation domain is a fusion of the carboxy terminus from the p65 subunit of NF-kappa B and FKBP12-rapamycin binding (FRB) domain of FKBP12-rapamycin-associated protein (FRAP). In one embodiment, the activation domain is a FKBP12-rapamycin binding (FRB) domain of human FKBP 12-rapamycin-associated protein (FRAP) fused to a carboxy terminus from the p65 subunit of NF-kappa B from a human.

In one embodiment, there is a linker between the transactivation domain and DNA binding domain, which linker may be an F2A or an IRES. In one embodiment the linker is selected from an IRES or a 2A peptide. In one embodiment, the linker is a cleavable 2A peptide. In one embodiment, the 2A peptide is selected to increase the packaging limit to allow for a single vector system.

The DNA binding domain is composed of a DNA-binding fusion of zinc finger homeodomain 1 (ZFHD1) joined to up to three copies of FK506 binding protein (FKBP). In the presence of an inducing agent, e.g., a rapalog such as rapamycin, the DNA binding domain and activation domain are dimerized through interaction of their FKBP and FRB domains, leading to transcription activation of the transgene. In some embodiments, the ZFHD1 is included in frame with the GT2A or IRES.

The expression system is designed to have one, two or three copies of the FKBP sequence. These are termed herein FKBP subunits. In one embodiment, the subunits are designed to express the same protein, but to have nucleic acids which are divergent from one another in order to minimize recombination. For example, SEQ ID NO: 33 provides 3 “wobbled” coding sequences for FKBP, each of which encode the sequence shown in SEQ ID NO: 34:

GVQVETISPGDGRTFPKRGQTCVVHYTGMLEDGKKFDSSRDRNKPFKFML
GKQEVIRGWEEGVAQMSVGQRAKLTISPDYAYGATGHPGIIPPHATLVFD
VELLKLE

The expression system further comprises zinc finger homeodomain binding sites. The nucleic acid molecule contains at least 1, 2, 3, 4, 5, 6, 7, 8, 9, 10, 11 or 12 binding sites for ZFHD. In one embodiment, the expression system contains 12 (twelve) zinc finger homeodomains binding site (binding partners) (12XZFHD). In another embodiment, the expression system contains 8 (eight) zinc finger homeodomains binding site (binding partners) (12XZFHD). However, the invention encompasses expression systems having from two to about twelve copies of the zinc finger binding site. An example of a single copy of a ZFHD binding site is: AATGATGGGCGCTCGAGT (SEQ ID NO: 35)

In some embodiments, there is a minimal IL2 promoter downstream of the zinc finger homeodomain binding sites. An exemplary IL2 promoter is shown in SEQ ID NO: 36.

Such inducible systems are known in the art, and include, e.g., the rapamycin-inducible system described by e.g., Rivera et al, A humanized system for pharmacologic control of gene expression, Nature Medicine volume 2, pages 1028-1032 (September 1996) and Rivera et al, Long-term pharmacologically regulated expression of erythropoietin in primates following AAV-mediated gene transfer, Blood, 15 Feb. 2005, volume 105, number 4, both of which are incorporated herein by reference. In one embodiment, the inducible gene expression system comprises a CMV promoter, the activation domain is a FKBP 12-rapamycin binding (FRB) domain of human FKBP12-rapamycin-associated protein (FRAP) fused to a carboxy terminus from the p65 subunit of NF-kappa B from a human, GT2A peptide. ZFHD1 DNA binding domain, three FKBP subunits, an hGH poly A, 12XZFHD, and a minimal sIL2 promoter. These sequences are in addition to the coding sequence for the PTH fusion protein and optionally other regulatory sequences.

In addition to a promoter, an expression cassette and/or a vector may contain other appropriate transcription initiation, termination, enhancer sequences, efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. Examples of suitable polyA sequences include, e.g., SV40, bovine growth hormone (bGH), human growth hormone (hGH), SV40, rabbit β-globin (also referred to as rabbit globin poly A; RGB), modified RGB (mRGB) and TK poly A. Examples of suitable enhancers include, e.g., the alpha fetoprotein enhancer, the TTR minimal promoter/enhancer, LSP (TH-binding globulin promoter/alphal-microglobulin/bikunin enhancer), amongst others. In one embodiment, the poly A is a rabbit globin poly A.

These control sequences are “operably linked” to the PTH construct sequences. As used herein, the term “operably linked” refers to both expression control sequences that are contiguous with the gene of interest and expression control sequences that act in trans or at a distance to control the gene of interest.

In one embodiment, a rAAV is provided which includes a 5′ ITR, CB7 promoter, chicken beta-actin intron, coding sequence for the fusion protein of SEQ ID NO: 1, 11, 13, 15, 17, 19, 21, 23, 25, 27, 29, 39, 42, 44, 46, or 48, a rabbit globin poly A, and a 3′ ITR. In another embodiment, the rAAV comprises a polynucleotide comprising a CMV promoter, the activation domain is a FKBP12-rapamycin binding (FRB) domain of human FKBP12-rapamycin-associated protein (FRAP) fused to a carboxy terminus from the p65 subunit of NF-kappa B from a human, IRES, ZFHD1 DNA binding domain, three FKBP subunits, an hGH poly A, 8XZFHD, a minimal sIL2 promoter, coding sequence for the PTH fusion protein of SEQ ID NO: 1, 11, 13, 15, 17, 19, 21, 23, 25, 27, 29, 39, 42, 44, 46, or 48 and rabbit beta globin polyA.

In another embodiment, a two-vector inducible system is provided. The first rAAV comprises 12XZFHD, a minimal IL2 promoter, coding sequence for the PTH fusion protein of SEQ ID NO: 1, 11, 13, 15, 17, 19, 21, 23, 25, 27, 29, 39, 42, 44, 46, or 48, and rabbit beta globin poly A. A stuffer sequence may be included to increase the packaging size of the vector. The second rAAV comprises a polynucleotide comprising a CMV promoter, an intron, the activation domain is a FKBP12-rapamycin binding (FRB) domain of human FKBP12-rapamycin-associated protein (FRAP) fused to a carboxy terminus from the p65 subunit of NF-kappa B from a human, IRES, ZFHD1 DNA binding domain, and a polyA.

In one embodiment, an expression cassette is provided that includes a polynucleotide comprising a CB7 promoter, chicken beta-actin intron, coding sequence for the fusion protein of SEQ ID NO: 1, 11, 13, 15, 17, 19, 21, 23, 25, 27, 29, 39, 42, 44, 46, or 48, and a rabbit globin poly A.

In another embodiment, an expression cassette is provided that includes a polynucleotide comprising a CB7 promoter, chicken beta-actin intron, coding sequence for the fusion protein of SEQ ID NO: 1, 11, 13, 15, 17, 19, 21, 23, 25, 27, 29, 39, 42, 44, 46, or 48, and a rabbit globin poly A.

### Viral Vectors

In another aspect, viral vectors that include the expression cassettes described herein are provided. In certain embodiments of the viral vectors described herein, the viral vector is an adeno-associated virus (AAV) viral vector or recombinant AAV (rAAV). The term “recombinant AAV” or “rAAV” as used herein refers to naturally occurring adeno-associated viruses, adeno-associated viruses available to one of skill in the art and/or in light of the composition(s) and method(s) described herein, as well as artificial AAVs. An adeno-associated virus (AAV) viral vector is an AAV DNase-resistant particle having an AAV protein capsid into which is packaged an expression cassette flanked by AAV inverted terminal repeat sequences (ITRs) (together referred to as the “vector genome”) for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. Various AAVs may be selected as sources for capsids of AAV viral vectors as identified above. In one embodiment, the AAV capsid is an AAVrh91 capsid or variant thereof. In certain embodiments, the capsid protein is designated by a number or a combination of numbers and letters following the term “AAV” in the name of the rAAV vector. Unless otherwise specified, the AAV capsid. ITRs, and other selected AAV components described herein, may be readily selected from among any AAV, including, without limitation, the AAVs identified as AAV1, AAV2, AAV3, AAV4, AAV5, AAV6, AAV7, AAV8, AAV9, AAVrh10, AAVhu37, AAVrh32.33, AAV Anc80, AAV10, AAV11, AAV12, AAVrh8, AAVrh74, AAV-DJ8, AAV-DJ, AAVhu.37, AAVrh.64R1, and AAVhu68. See, e.g., US Published Patent Application No. 2007-0036760-A1; US Published Patent Application No. 2009-0197338-A1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, and WO 2003/042397 (rh.10), WO 2005/033321, WO 2018/160582 (AAVhu68), which are incorporated herein by reference. Other suitable AAVs may include, without limitation, AAVrh90 [PCT/US20/30273, filed Apr. 28, 2020], AAVrh91 [PCT/US20/030266, filed Apr. 28, 2020, now a publication WO 2020/223231, published Nov. 5, 2020], AAVrh92, AAVrh93, AAVrh91.93 [PCT/US20/30281, filed Apr. 28, 2020], which are incorporated by reference herein. Other suitable AAV include AAV3B variants which are described in US Provisional Patent Application No. 62/924,112, filed Oct. 21, 2019, and U.S. Provisional Patent Application No. 63/025,753, filed May 15, 2020, describing AAV3B.AR2.01, AAV3B.AR2.02, AAV3B.AR2.03, AAV3B.AR2.04, AAV3B.AR2.05, AAV3B.AR2.06,AAV3B.AR2.07, AAV3B.AR2.08, AAV3B.AR2.10, AAV3B.AR2.11, AAV3B.AR2.12,AAV3B.AR2.13, AAV3B.AR2.14, AAV3B.AR2.15, AAV3B.AR2.16, or AAV3B.AR2.17,which are incorporated herein by reference. See also, International Patent Application No. PCT/US21/45945, filed Aug. 13, 2021, U.S. Provisional Patent Application No. 63/065,616, filed Aug. 14, 2020, and U.S. Provisional Patent Application No. 63/109,734, filed Nov. 4, 2020, which are all incorporated herein by reference in its entireties. These documents also describe other AAV capsids which may be selected for generating rAAV and are incorporated by reference. Among the AAVs isolated or engineered from human or non-human primates (NHP) and well characterized, human AAV2 is the first AAV that was developed as a gene transfer vector; it has been widely used for efficient gene transfer experiments in different target tissues and animal models.

As used herein, relating to AAV, the term “variant” means any AAV sequence which is derived from a known AAV sequence, including those with a conservative amino acid replacement, and those sharing at least 90%, at least 95%, at least 97%, at least 99% or greater sequence identity over the amino acid or nucleic acid sequence. In another embodiment, the AAV capsid includes variants which may include up to about 10% variation from any described or known AAV capsid sequence. That is, the AAV capsid shares about 90% identity to about 99.9% identity, about 95% to about 99% identity or about 97% to about 98% identity to an AAV capsid provided herein and/or known in the art. In one embodiment, the AAV capsid shares at least 95% identity with an AAV capsid. When determining the percent identity of an AAV capsid, the comparison may be made over any of the variable proteins (e.g., vp1, vp2, or vp3).

In one embodiment, the viral vector is an rAAV having the capsid of AAV8 or a functional variant thereof. In one embodiment, the viral vector is an rAAV having the capsid of AAVrh91 or a functional variant thereof. In one embodiment, the viral vector is an rAAV having the capsid of AAV3.AR.2.12 or a functional variant thereof. In one embodiment, the viral vector is an rAAV having a capsid selected from AAV9, AAVrh64R1, AAVhu37, or AAVrh10. In one embodiment, the viral vector is an rAAV having the capsid of AAVhu68 or a functional variant thereof.

In certain embodiments, the viral vector has an AAVrh91 capsid. A nucleic acid sequence encoding the AAVrh91 capsid is provided in SEQ ID NO: 37 and the encoded amino acid sequence is provided in SEQ ID NO: 38. Provided herein is an rAAV comprising at least one of the vp1, vp2 and the vp3 of AAVrh91 (SEQ ID NO: 38). Also provided herein are rAAV comprising an AAV capsid encoded by at least one of the vp1, vp2 and the vp3 of AAVrh91 (SEQ ID NO: 37). In yet another embodiment, a nucleic acid sequence encoding the AAVrh91 amino acid sequence is provided in SEQ ID NO: 19 and the encoded amino acid sequence is provided in SEQ ID NO: 38. Also provided herein are rAAV comprising an AAV capsid encoded by at least one of the vp1, vp2 and the vp3 of AAVrh91eng (SEQ ID NO: 19). In certain embodiments, the vp1, vp2 and/or vp3 is the full-length capsid protein of AAVrh91 (SEQ ID NO: 38). In other embodiments, the vp1, vp2 and/or vp3 has an N-terminal and/or a C-terminal truncation (e.g., truncation(s) of about 1 to about 10 amino acids).

In certain embodiments, an AAVrh91 capsid is characterized by one or more of the following: (1) AAVrh91 capsid proteins comprising: a heterogeneous population of AAVrh91 vp1 proteins selected from: vp1 proteins produced by expression from a nucleic acid sequence which encodes the predicted amino acid sequence of 1 to 736 of SEQ ID NO: 38, vp1 proteins produced from SEQ ID NO: 37, or vp1 proteins produced from a nucleic acid sequence at least 70% identical to SEQ ID NO: 37 which encodes the predicted amino acid sequence of 1 to 736 of SEQ ID NO: 38, a heterogeneous population of AAVrh91 vp2 proteins selected from: vp2 proteins produced by expression from a nucleic acid sequence which encodes the predicted amino acid sequence of at least about amino acids 138 to 736 of SEQ ID NO: 38, vp2 proteins produced from a sequence comprising at least nucleotides 412 to 2208 of SEQ ID NO: 37, or vp2 proteins produced from a nucleic acid sequence at least 70% identical to at least nucleotides 412 to 2208 of SEQ ID NO: 37 which encodes the predicted amino acid sequence of at least about amino acids 138 to 736 of SEQ ID NO: 38, a heterogeneous population of AAVrh91 vp3 proteins selected from: vp3 proteins produced by expression from a nucleic acid sequence which encodes the predicted amino acid sequence of at least about amino acids 203 to 736 of SEQ ID NO: 38, vp3 proteins produced from a sequence comprising at least nucleotides 607 to 2208 of SEQ ID NO: 37, or vp3 proteins produced from a nucleic acid sequence at least 70% identical to at least nucleotides 607 to 2208 of SEQ ID NO: 37 which encodes the predicted amino acid sequence of at least about amino acids 203 to 736 of SEQ ID NO: 38; and/or (2) a heterogeneous population of vp1 proteins which are the product of a nucleic acid sequence encoding the amino acid sequence of SEQ ID NO: 38, a heterogeneous population of vp2 proteins which are the product of a nucleic acid sequence encoding the amino acid sequence of at least about amino acids 138 to 736 of SEQ ID NO: 38, and a heterogeneous population of vp3 proteins which are the product of a nucleic acid sequence encoding at least amino acids 203 to 736 of SEQ ID NO: 38, wherein: the vp1, vp2 and vp3 proteins contain subpopulations with amino acid modifications comprising at least two highly deamidated asparagines (N) in asparagine-glycine pairs in SEQ ID NO: 38 and optionally further comprising subpopulations comprising other deamidated amino acids, wherein the deamidation results in an amino acid change; and (B) a vector genome in the AAVrh91 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, an AAVrh91 capsid is characterized by one or more of the following: (1) AAVrh91 capsid proteins comprising: a heterogeneous population of AAVrh91 vp1 proteins selected from: vp1 proteins produced by expression from a nucleic acid sequence which encodes the predicted amino acid sequence of 1 to 736 of SEQ ID NO: 38, vp1 proteins produced from SEQ ID NO: 19, or vp1 proteins produced from a nucleic acid sequence at least 70% identical to SEQ ID NO: 19 which encodes the predicted amino acid sequence of 1 to 736 of SEQ ID NO: 38, a heterogeneous population of AAVrh91 vp2 proteins selected from: vp2 proteins produced by expression from a nucleic acid sequence which encodes the predicted amino acid sequence of at least about amino acids 138 to 736 of SEQ ID NO: 38, vp2 proteins produced from a sequence comprising at least nucleotides 412 to 2208 of SEQ ID NO: 19, or vp2 proteins produced from a nucleic acid sequence at least 70% identical to at least nucleotides 412 to 2208 of SEQ ID NO: 19 which encodes the predicted amino acid sequence of at least about amino acids 138 to 736 of SEQ ID NO: 38, a heterogeneous population of AAVrh91 vp3 proteins selected from: vp3 proteins produced by expression from a nucleic acid sequence which encodes the predicted amino acid sequence of at least about amino acids 203 to 736 of SEQ ID NO: 38, vp3 proteins produced from a sequence comprising at least nucleotides 607 to 2208 of SEQ ID NO: 19, or vp3 proteins produced from a nucleic acid sequence at least 70% identical to at least nucleotides 607 to 2208 of SEQ ID NO: 19 which encodes the predicted amino acid sequence of at least about amino acids 203 to 736 of SEQ ID NO: 38; and/or (2) a heterogeneous population of vp1 proteins which are the product of a nucleic acid sequence encoding the amino acid sequence of SEQ ID NO: 38, a heterogeneous population of vp2 proteins which are the product of a nucleic acid sequence encoding the amino acid sequence of at least about amino acids 138 to 736 of SEQ ID NO: 38, and a heterogeneous population of vp3 proteins which are the product of a nucleic acid sequence encoding at least amino acids 203 to 736 of SEQ ID NO: 38, wherein: the vp1, vp2 and vp3 proteins contain subpopulations with amino acid modifications comprising at least two highly deamidated asparagines (N) in asparagine-glycine pairs in SEQ ID NO: 38 and optionally further comprising subpopulations comprising other deamidated amino acids, wherein the deamidation results in an amino acid change; and (B) a vector genome in the AAVrh91 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, the AAVrh91 vp1, vp2 and vp3 proteins contain subpopulations with amino acid modifications comprising at least two highly deamidated asparagines (N) in asparagine-glycine pairs in SEQ ID NO: 38 and optionally further comprising subpopulations comprising other deamidated amino acids, wherein the deamidation results in an amino acid change. High levels of deamidation at N-G pairs N57, N383 and/or N512 are observed, relative to the number of SEQ ID NO: 38. Deamidation has been observed in other residues. In certain embodiments, AAVrh91 may have other residues deamidated, e.g., typically at less than 10% and/or may have other modifications, including phosphorylation (e.g., where present, in the range of about 2 to about 30%, or about 2 to about 20%, or about 2 to about 10%) (e.g., at S149), or oxidation (e.g, at one or more of ˜W22, ˜M211, W247, M403, M435, M471, W478, W503, ˜M537, ˜M541, ˜M559, ˜M599, M635, and/or, W695). Optionally the W may oxidize to kynurenine.

TABLE A
AAVrh91 Deamidation

| AAVrh91 | |
|---|---|
| Deamidation based | |
| on VP1 numbering | % Deamidation |
| N57 + Deamidation | 65-90, 70-95, 80-95, 75-100, 80-100, or 90-100 |
| N94 + Deamidation | 2-15 or 2-5 |
| N303 + Deamidation | 2-15 or 5-10 |
| N383 + Deamidation | 65-90, 70-95, 80-95, 75-100, 80-100, or 90-100 |
| N497 + Deamidation | 2-15 or 5-10 |
| N512 + Deamidation | 65-90, 70-95, 80-95, 75-100, 80-100, or 90-100 |
| ~N691 + Deamidation | 2-15, 2-10, or 5-10 |

In certain embodiments, an AAVrh91 capsid is modified in one or more of the positions identified in the preceding table, in the ranges provided, as determined using mass spectrometry with a trypsin enzyme. In certain embodiments, one or more of the positions, or the glycine following the N is modified as described herein. Residue numbers are based on the AAVrh91 sequence provided herein. See, SEQ ID NO: 38.

In certain embodiments, an AAVrh91 capsid comprises: a heterogeneous population of vp1 proteins which are the product of a nucleic acid sequence encoding the amino acid sequence of SEQ ID NO: 38, a heterogeneous population of vp2 proteins which are the product of a nucleic acid sequence encoding the amino acid sequence of at least about amino acids 138 to 736 of SEQ ID NO: 38, and a heterogeneous population of vp3 proteins which are the product of a nucleic acid sequence encoding at least amino acids 203 to 736 of SEQ ID NO: 38.

In certain embodiments, the modified AAVrh91 nucleic acid sequence is be used to generate a mutant rAAV having a capsid with lower deamidation than the native AAVrh91 capsid. Such mutant rAAV may have reduced immunogenicity and/or increase stability on storage, particularly storage in suspension form.

In one aspect, a recombinant AAV (rAAV) is provided. The rAAV includes an AAV capsid from adeno-associated virus rh91, and a vector genome packaged in the AAV capsid, said vector genome comprising AAV inverted terminal repeats (ITRs), a coding sequence for the PTH fusion of SEQ ID NO: 1, 11, 13, 15, 17, 19, 21, 23, 25, 27. 29, 39, 42, 44, 46, or 48, and regulatory sequences which direct expression of the PTH fusion.

In certain embodiments, an AAV68 capsid is further characterized by one or more of the following. AAV hu68 capsid proteins comprise: AAVhu68 vp1 proteins produced by expression from a nucleic acid sequence which encodes the predicted amino acid sequence of 1 to 736 of SEQ ID NO: 100, vp1 proteins produced from SEQ ID NO: 98 or 99, or vp1 proteins produced from a nucleic acid sequence at least 70% identical to SEQ ID NO: 98 which encodes the predicted amino acid sequence of 1 to 736 of SEQ ID NO: 100; AAVhu68 vp2 proteins produced by expression from a nucleic acid sequence which encodes the predicted amino acid sequence of at least about amino acids 138 to 736 of SEQ ID NO: 100, vp2 proteins produced from a sequence comprising at least nucleotides 412 to 2211 of SEQ ID NO: 98 or 99, or vp2 proteins produced from a nucleic acid sequence at least 70% identical to at least nucleotides 412 to 2211 of SEQ ID NO: 98 or 99 which encodes the predicted amino acid sequence of at least about amino acids 138 to 736 of SEQ ID NO: 100, and/or AAVhu68 vp3 proteins produced by expression from a nucleic acid sequence which encodes the predicted amino acid sequence of at least about amino acids 203 to 736 of SEQ ID NO: 100, vp3 proteins produced from a sequence comprising at least nucleotides 607 to 2211 of SEQ ID NO: 98, or vp3 proteins produced from a nucleic acid sequence at least 70% identical to at least nucleotides 607 to 2211 of SEQ ID NO: 98 or 99 which encodes the predicted amino acid sequence of at least about amino acids 203 to 736 of SEQ ID NO: 100.

Additionally or alternatively, an AAV capsid is provided which comprise a heterogenous population of vp1 proteins optionally comprising a valine at position 157, a heterogenous population of vp2 proteins optionally comprising a valine at position 157, and a heterogenous population of vp3 proteins, wherein at least a subpopulation of the vp1 and vp2 proteins comprise a valine at position 157 and optionally further comprising a glutamic acid at position 67 based on the numbering of the vp1 capsid of SEQ ID NO: 100. Additionally or alternatively, an AAVhu68 capsid is provided which comprises a heterogenous population of vp1 proteins which are the product of a nucleic acid sequence encoding the amino acid sequence of SEQ ID NO: 100, a heterogenous population of vp2 proteins which are the product of a nucleic acid sequence encoding the amino acid sequence of at least about amino acids 138 to 736 of SEQ ID NO: 100, and a heterogenous population of vp3 proteins which are the product of a nucleic acid sequence encoding at least amino acids 203 to 736 of SEQ ID NO: 100, wherein: the vp1, vp2 and vp3 proteins contain subpopulations with amino acid modifications

The AAVhu68 vp1, vp2 and vp3 proteins are typically expressed as alternative splice variants encoded by the same nucleic acid sequence which encodes the full-length vp1 amino acid sequence of SEQ ID NO: 100 (amino acid 1 to 736). Optionally the vp1-encoding sequence is used alone to express the vp1, vp2 and vp3 proteins. Alternatively, this sequence may be co-expressed with one or more of a nucleic acid sequence which encodes the AAVhu68 vp3 amino acid sequence of SEQ ID NO: 100 (about aa 203 to 736) without the vp1-unique region (about aa 1 to about aa 137) and/or vp2-unique regions (about aa 1 to about aa 202), or a strand complementary thereto, the corresponding mRNA or tRNA (about nt 607 to about nt 2211 of SEQ ID NO: 98 or 99), or a sequence at least 70% to at least 99% (e.g., at least 85%, at least 90%, at least 95%, at least 97%, at least 98% or at least 99%) identical to SEQ ID NO: 98 or 99 which encodes aa 203 to 736 of SEQ ID NO: 100. Additionally, or alternatively, the vp1-encoding and/or the vp2-encoding sequence may be co-expressed with the nucleic acid sequence which encodes the AAVhu68 vp2 amino acid sequence of SEQ ID NO: 100 (about aa 138 to 736) without the vp1-unique region (about aa 1 to about 137), or a strand complementary thereto, the corresponding mRNA or tRNA (nt 412 to 22121 of SEQ ID NO: 98), or a sequence at least 70% to at least 99% (e.g., at least 85%, at least 90%, at least 95%, at least 97%, at least 98% or at least 99%) identical to SEQ ID NO: 98 or 99 which encodes about aa 138 to 736 of SEQ ID NO: 100.

As described herein, a rAAVhu68 has a rAAVhu68 capsid produced in a production system expressing capsids from an AAVhu68 nucleic acid which encodes the vp1 amino acid sequence of SEQ ID NO: 100, and optionally additional nucleic acid sequences, e.g., encoding a vp3 protein free of the vp1 and/or vp2-unique regions. The rAAVhu68 resulting from production using a single nucleic acid sequence vp1 produces the heterogenous populations of vp1 proteins, vp2 proteins and vp3 proteins. More particularly, the AAVhu68 capsid contains subpopulations within the vp1 proteins, within the vp2 proteins and within the vp3 proteins which have modifications from the predicted amino acid residues in SEQ ID NO: 100. These subpopulations include, at a minimum, deamidated asparagine (N or Asn) residues. For example, asparagines in asparagine-glycine pairs are highly deamidated.

In one embodiment, the AAVhu68 vp1 nucleic acid sequence has the sequence of SEQ ID NO: 98 or 99, or a strand complementary thereto, e.g., the corresponding mRNA or tRNA. In certain embodiments, the vp2 and/or vp3 proteins may be expressed additionally or alternatively from different nucleic acid sequences than the vp1, e.g., to alter the ratio of the vp proteins in a selected expression system. In certain embodiments, also provided is a nucleic acid sequence which encodes the AAVhu68 vp3 amino acid sequence of SEQ ID NO: 100 (about aa 203 to 736) without the vp1-unique region (about aa 1 to about aa 137) and/or vp2-unique regions (about aa 1 to about aa 202), or a strand complementary thereto, the corresponding mRNA or tRNA (about nt 607 to about nt 2211 of SEQ ID NO: 98 or 99). In certain embodiments, also provided is a nucleic acid sequence which encodes the AAVhu68vp2 amino acid sequence of SEQ ID NO: 100 (about aa 138 to 736) without the vp1-unique region (about aa 1 to about 137), or a strand complementary thereto, the corresponding mRNA or tRNA (nt 412 to 2211 of SEQ ID NO: 98 or 99).

However, other nucleic acid sequences which encode the amino acid sequence of SEQ ID NO: 100 may be selected for use in producing rAAVhu68 capsids. In certain embodiments, the nucleic acid sequence has the nucleic acid sequence of SEQ ID NO: 98 or 99 or a sequence at least 70% to 99% identical, at least 75%, at least 80%, at least 85%, at least 90%, at least 95%, at least 97%, at least 99%, identical to SEQ ID NO: 98 or 99 which encodes SEQ ID NO: 100. In certain embodiments, the nucleic acid sequence has the nucleic acid sequence of SEQ ID NO: 98 or 99 or a sequence at least 70% to 99. %, at least 75%, at least 80%, at least 85%, at least 90%, at least 95%, at least 97%, at least 99%, identical to about nt 412 to about nt 2211 of SEQ ID NO: 98 or 99 which encodes the vp2 capsid protein (about aa 138 to 736) of SEQ ID NO: 100. In certain embodiments, the nucleic acid sequence has the nucleic acid sequence of about nt 607 to about nt 2211 of SEQ ID NO: 98 or 99 or a sequence at least 70% to 99. %, at least 75%, at least 80%, at least 85%, at least 90%, at least 95%, at least 97%, at least 99%, identical to nt SEQ ID NO: 98 or 99 which encodes the vp3 capsid protein (about aa 203 to 736) of SEQ ID NO: 100.

| Deamidation | | |
|---|---|---|
| Based on Predicted AAVHu68 | Average % Based on VP1/VP2/VP3 Proteins in | |
| [SEQ ID NO: 100] | AAVhu68 Capsid | |
| Deamidated Residue + 1 | Broad Range of | |
| (Neighboring AA) | Percentages (%) | Narrow Ranges (%) |
| N57 | 78 to 100% | 80 to 100, 85 to 97 |
| (N-G) | | |
| N66 | 0 to 5 | 0, 1 to 5 |
| (N-E) | | |
| N94 | 0 to 15, | 0, 1 to 15, 5 to 12, 8 |
| (N-H) | | |
| N113 | 0 to 2 | 0, 1 to 2 |
| (N-L) | | |
| ~N253 | 10 to 25 | 15 to 22 |
| (N-N) | | |
| Q259 | 8 to 42 | 10 to 40, 20 to 35 |
| (Q-I) | | |
| ~N270 | 12 to 30 | 15 to 28 |
| (N-D) | | |
| ~N304 | 0 to 5 | 1 to 4 |
| (N-N) (position 303 also N) | | |
| N319 | 0 to 5 | 0, 1 to 5, 1 to 3 |
| (N-I) | | |
| N329 * | 65 to 100 | 70 to 95, 85 to 95, 80 to |
| (N-G)*(position 328 also N) | | 100, 85 to 100, |
| N336 | 0 to 100 | 0, 1 to 10, 25 to 100, 30 to |
| (N-N) | | 100, 30 to 95 |
| ~N409 | 15 to 30 | 20 to 25 |
| (N-N) | | |
| N452 | 75 to 100 | 80 to 100, 90 to 100, 95 to |
| (N-G) | | 100, |
| N477 | 0 to 8 | 0, 1 to 5 |
| (N-Y) | | |
| N512 | 65 to 100 | 70 to 95, 85 to 95, 80 to |
| (N-G) | | 100, 85 to 100, |
| ~N515 | 0 to 25 | 0, 1 to 10, 5 to 25, 15 to 25 |
| (N-S) | | |
| ~Q599 | 1 to 20 | 2 to 20, 5 to 15 |
| (Asn-Q-Gly) | | |
| N628 | 0 to 10 | 0, 1 to 10, 2 to 8 |
| (N-F) | | |
| N651 | 0 to 3 | 0, 1 to 3 |
| (N-T) | | |
| N663 | 0 to 5 | 0, 1 to 5, 2 to 4 |
| (N-K) | | |
| N709 | 0 to 25 | 0, 1 to 22, 15 to 25 |
| (N-N) | | |
| N735 | 0 to 40 | 0. 1 to 35, 5 to 50, 20 to 35 |

In certain embodiments, the AAVhu68 capsid is characterized, by having, capsid proteins in which at least 45% of N residues are deamidated at least one of positions N57, N329, N452, and/or N512 based on the numbering of amino acid sequence of SEQ ID NO: 100. In certain embodiments, at least about 60%, at least about 70%, at least about 80%, or at least 90% of the N residues at one or more of these N-G positions (i.e., N57, N329, N452, and/or N512, based on the numbering of amino acid sequence of SEQ ID NO: 100) are deamidated. In these and other embodiments, an AAVhu68 capsid is further characterized by having a population of proteins in which about 1% to about 20% of the N residues have deamidations at one or more of positions: N94, N253, N270, N304, N409, N477, and/or Q599, based on the numbering of amino acid sequence of SEQ ID NO: 100. In certain embodiments, the AAVhu68 comprises at least a subpopulation of vp1, vp2 and/or vp3 proteins which are deamidated at one or more of positions N35, N57, N66, N94, N113, N252, N253, Q259, N270, N303, N304, N305, N319, N328, N329, N336, N409, N410, N452, N477, N515, N598, Q599, N628, N651, N663, N709, N735, based on the numbering of amino acid sequence of SEQ ID NO: 100, or combinations thereof. In certain embodiments, the capsid proteins may have one or more amidated amino acids.

In one aspect, a recombinant AAV (rAAV) is provided. The rAAV includes an AAV capsid from adeno-associated virus hu68, and a vector genome packaged in the AAV capsid, said vector genome comprising AAV inverted terminal repeats (ITRs), a coding sequence for the PTH fusion of SEQ ID NO: 1, 11, 13, 15, 17, 19, 21, 23, 25, 27, 29, 39, 42, 44, 46, or 48, and regulatory sequences which direct expression of the PTH fusion.

In another embodiment a recombinant adeno-associated virus (rAAV) is provided that has an AAVhu68 capsid and a vector genome, wherein (a) the AAV hu68 capsid comprises a heterogenous population of AAVhu68 vp1 proteins, a heterogenous population of AAVhu68 vp2 proteins; and a heterogenous population of AAVhu68 vp3 proteins, wherein the heterogenous AAVhu68 vp1, AAVhu68 vp2 and AAVhu68 vp3 proteins contain subpopulations with amino acid modifications comprising 50% to 100% deamidation in at least two asparagines (N) in asparagine-glycine pairs in two or more of N57, N329, N452, N512 of SEQ ID NO: 100 as determined using mass spectrometry and optionally further comprising subpopulations comprising other deamidated amino acids, wherein the deamidation results in an amino acid change, wherein the deamidated asparagines are deamidated to aspartic acid, isoaspartic acid, an interconverting aspartic acid/isoaspartic acid pair, or combinations thereof, wherein the AAVhu68 capsid further comprises subpopulations having one or more of:
(a) at least 65% of asparagines (N) in asparagine-glycine pairs located at positions N57 of the vp1 proteins are deamidated, based on the numbering of SEQ ID NO: 100;
(b) at least 75% of N in asparagine-glycine pairs in position N329 of the vp1, v2 and vp3 proteins are deamidated, based on the residue numbering of the amino acid sequence of SEQ ID NO: 100,
(c) at least 50% of N in asparagine-glycine pairs in position N452 of the vp1, v2 and vp3 proteins are deamidated, based on the residue numbering of the amino acid sequence of SEQ ID NO: 100; and/or
(d) at least 75% of N in asparagine-glycine pairs in position N512 of the vp1, v2 and vp3 proteins are deamidated, based on the residue numbering of the amino acid sequence of SEQ ID NO: 100, and
   a vector genome in the AAVhu68 capsid, the vector genome comprising a nucleic acid molecule comprising AAV inverted terminal repeat sequences and a non-AAV nucleic acid sequence encoding a PTH fusion as described herein operably linked to sequences which direct expression of PTH fusion in a target cell.

Still other modifications are observed, most of which do not result in conversion of one amino acid to a different amino acid residue. Optionally, at least one Lys in the vp1, vp2 and vp3 of the capsid are acetylated. Optionally, at least one Asp in the vp1, vp2 and/or vp3 of the capsid is isomerized to D-Asp. Optionally, at least one S (Ser, Serine) in the vp1, vp2 and/or vp3 of the capsid is phosphorylated. Optionally, at least one T (Thr, Threonine) in the vp1, vp2 and/or vp3 of the capsid is phosphory lated. Optionally, at least one W (trp, tryptophan) in the vp1, vp2 and/or vp3 of the capsid is oxidized. Optionally, at least one M (Met, Methionine) in the vp1, vp2 and/or vp3 of the capsid is oxidized. In certain embodiments, the capsid proteins have one or more phosphory lations. For example, certain vp1 capsid proteins may be phosphorylated at position 149.

In one embodiment, the rAAV is an scAAV. The abbreviation “sc” refers to self-complementary. “Self-complementary AAV” refers a plasmid or vector having an expression cassette in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

In one embodiment, the nucleic acid sequences encoding the PTH constructs described herein are engineered into any suitable genetic element, e.g., naked DNA, phage, transposon, cosmid, RNA molecule (e.g., mRNA), episome, etc., which transfers the PTH sequences carried thereon to a host cell, e.g., for generating nanoparticles carrying DNA or RNA, viral vectors in a packaging host cell and/or for delivery to a host cell in a subject. In one embodiment, the genetic element is a plasmid. The selected genetic element may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

As used herein, the term “host cell” may refer to the packaging cell line in which a vector (e.g., a recombinant AAV or rAAV) is produced from a production plasmid. In the alternative, the term “host cell” may refer to any target cell in which expression of a gene product described herein is desired. Thus, a “host cell,” refers to a prokaryotic or eukaryotic cell (e.g., bacterial cell, human cell or insect cell) that contains exogenous or heterologous DNA that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. In certain embodiments herein, the term “host cell” refers to cultures of cells of various mammalian species for in vitro assessment of the compositions described herein. In other embodiments herein, the term “host cell” refers to the cells employed to generate and package the viral vector or recombinant virus. In a further embodiment, the term “host cell” is an intestine cell, a small intestine cell, a pancreatic cell, a liver cell.

As used herein, the term “target cell” refers to any target cell in which expression of a heterologous nucleic acid sequence or protein is desired. In certain embodiments, the target cell is a liver cell. In other embodiments, the target cell is a muscle cell.

In one embodiment, the rAAV is provided which comprises a vector genome comprising an expression cassette, wherein the expression cassette comprises a CMV promoter, the activation domain is a FKBP12-rapamycin binding (FRB) domain of human FKBP12-rapamycin-associated protein (FRAP) fused to a carboxy terminus from the p65 subunit of NF-kappa B from a human, GT2A_V1 peptide, ZFHD1 DNA binding domain, three FKBP subunits, an hGH poly A, 12XZFHD, a minimal sIL2 promoter, coding sequence for the PTH fusion protein of SEQ ID NO: 14, and rabbit beta globin polyA. In another embodiment, the rAAV is provide which comprises a vector genome comprising an expression cassette, wherein the expression cassette comprises a CMV promoter, the activation domain is a FKBP12-rapamycin binding (FRB) domain of human FKBP12-rapamycin-associated protein (FRAP) fused to a carboxy terminus from the p65 subunit of NF-kappa B from a human, GT2A_V2 peptide, ZFHD1 DNA binding domain, three FKBP subunits, an hGH poly A, 12XZFHD, a minimal sIL2 promoter, coding sequence for the PTH fusion protein of SEQ ID NO: 14, and rabbit beta globin polyA.

In one embodiment, the rAAV is provided which comprises a vector genome comprising an expression cassette, wherein the expression cassette comprises a CB7 promoter, chicken beta-actin intron, coding sequence for the fusion protein of SEQ ID NO: 1, 11, 13, 15, 17, 19, 21, 23, 25, 27, 29, 39, 42, 44, 46, or 48, a rabbit globin poly A, and a 3′ ITR. In another embodiment, the rAAV is provided which comprises a vector genome comprising an expression cassette, wherein the expression cassette comprises a CMV promoter, the activation domain is a FKBP 12-rapamycin binding (FRB) domain of human FKBP12-rapamycin-associated protein (FRAP) fused to a carboxy terminus from the p65 subunit of NF-kappa B from a human, IRES, ZFHD1 DNA binding domain, three FKBP subunits, an hGH poly A, 8XZFHD, a minimal sIL2 promoter, coding sequence for the PTH fusion protein of SEQ ID NO: 1, 11, 13, 15, 17, 19, 21, 23, 25, 27, 29, 39, 42, 44, 46, or 48, and rabbit beta globin poly A.

In another embodiment, a two vector inducible system is provided. In one embodiment, the first rAAV is provided which comprises a vector genome comprising an expression cassette, wherein the expression cassette comprises a 12XZFHD, a minimal IL2 promoter, coding sequence for the PTH fusion protein of SEQ ID NO: 1, 11, 13, 15, 17, 19, 21, 23, 25, 27, 29, 39, 42, 44, 46, or 48, and rabbit beta globin polyA. A stuffer sequence may be included to increase the packaging size of the vector. The second rAAV comprises a vector genome comprising an expression cassette, wherein the expression cassette comprises a polynucleotide comprising a CMV promoter, an intron, the activation domain is a FKBP12-rapamycin binding (FRB) domain of human FKBP12-rapamycin-associated protein (FRAP) fused to a carboxy terminus from the p65 subunit of NF-kappa B from a human, IRES, ZFHD1 DNA binding domain, and a polyA.

In one embodiment, an rAAV includes a vector genome that includes an expression cassette that includes a polynucleotide comprising a CB7 promoter, chicken beta-actin intron, coding sequence for the fusion protein of SEQ ID NO: 1, 11, 13, 15, 17, 19, 21, 23, 25, 27, 29, 39, 42, 44, 46, or 48, and a rabbit globin poly A.

The minimal sequences required to package the expression cassette into an AAV viral particle are the AAV 5′ and 3′ ITRs, which may be of the same AAV origin as the capsid, or of a different AAV origin (to produce an AAV pseudotype). In one embodiment, the ITR sequences from AAV2, or the deleted version thereof (AITR), are used for convenience and to accelerate regulatory approval. However, ITRs from other AAV sources may be selected. Preferably, the source of the ITRs is the same as the source of the Rep protein, which is provided in trans for production. Typically, an expression cassette for an AAV vector comprises an AAV 5′ ITR, the PTH fusion protein coding sequences and any regulatory sequences, and an AAV 3′ ITR. However, other configurations of these elements may be suitable. A shortened version of the 5′ ITR, termed JITR, has been described in which the D-sequence and terminal resolution site (trs) are deleted. In other embodiments, the full-length AAV 5′ and 3′ ITRs are used.

For packaging an expression cassette into virions, the ITRs are the only AAV components required in cis in the same construct as the gene. In one embodiment, the coding sequences for the replication (rep) and/or capsid (cap) are removed from the AAV genome and supplied in trans or by a packaging cell line in order to generate the AAV vector. For example, as described above, a pseudotyped AAV may contain ITRs from a source which differs from the source of the AAV capsid. In one embodiment, a chimeric AAV capsid may be utilized. Still other AAV components may be selected. Sources of such AAV sequences are described herein and may also be isolated or obtained from academic, commercial, or public sources (e.g., the American Type Culture Collection, Manassas, VA). The AAV sequences may be obtained through synthetic or other suitable means by reference to published sequences such as are available in the literature or in databases such as, e.g., GenBank®, PubMed®, or the like.

Methods for generating and isolating AAV viral vectors suitable for delivery to a subject are known in the art. See, e.g., U.S. Pat. Nos. 7,790,449; 7,282,199; WO 2003/042397; WO 2005/033321, WO 2006/110689; and U.S. Pat. No. 7,588,772 B2]. In a one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV-the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065. See generally, e.g., Grieger & Samulski, 2005, “Adeno-associated virus as a gene therapy vector: Vector development, production and clinical applications,” Adv. Biochem. Engin/Biotechnol. 99: 119-145; Buning et al., 2008, “Recent developments in adeno-associated virus vector technology,” J. Gene Med. 10:717-733; and the references cited below, each of which is incorporated herein by reference in its entirety. The methods used to construct any embodiment of this invention are known to those with skill in nucleic acid manipulation and include genetic engineering, recombinant engineering, and synthetic techniques. See, e.g., Green and Sambrook et al, Molecular Cloning: A Laboratory Manual, Cold Spring Harbor Press, Cold Spring Harbor, NY (2012). Similarly, methods of generating rAAV virions are well known and the selection of a suitable method is not a limitation on the present invention. See, e.g., K. Fisher et al. (1993) J. Virol., 70:520-532 and U.S. Pat. No. 5,478,745.

The rAAV described herein comprise a selected capsid with a vector genome packaged inside. The vector genome (or rAAV genome) comprises 5′ and 3′ AAV inverted terminal repeats (ITRs), the polynucleotide sequence encoding the fusion protein, and regulatory sequences which direct insertion of the polynucleotide sequence encoding the fusion protein to the genome of a host cell.

As used herein, a “vector genome” refers to the nucleic acid sequence packaged inside a parvovirus (e.g., rAAV) capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, coding sequence(s) (i.e., transgene(s)), and an AAV 3′ ITR. ITRs from AAV2, a different source AAV than the capsid, or other than full-length ITRs may be selected. In certain embodiments, the ITRs are from the same AAV source as the AAV which provides the rep function during production or a transcomplementing AAV. Further, other ITRs, e.g., self-complementary (scAAV) ITRs, may be used. Both single-stranded AAV and self-complementary (sc) AAV are encompassed with the rAAV. The transgene is a nucleic acid coding sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue. Suitable components of a vector genome are discussed in more detail herein. In one example, a “vector genome” contains, at a minimum, from 5° to 3°, a vector-specific sequence, a nucleic acid sequence encoding PTH constructs operably linked to regulatory control sequences (which direct their expression in a target cell), where the vector-specific sequence may be a terminal repeat sequence which specifically packages the vector genome into a viral vector capsid or envelope protein. For example, AAV inverted terminal repeats are utilized for packaging into AAV and certain other parvovirus capsids.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. Preferably, substantially the entire sequences encoding the ITRs are used in the molecule. although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J. Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. In one embodiment, the ITRs are from an AAV different than that supplying a capsid. In one embodiment, the ITR sequences from AAV2. However, ITRs from other AAV sources may be selected. A shortened version of the 5″ ITR. termed MITR, has been described in which the D-sequence and terminal resolution site (trs) are deleted. In certain embodiments, the vector genome includes a shortened AAV2 ITR of 130 base pairs, wherein the external A elements is deleted. Without wishing to be bound by theory, it is believed that the shortened ITR reverts back to the wild-type length of 145 base pairs during vector DNA amplification using the internal (A′) element as a template. In other embodiments, full-length AAV 5′ and 3′ ITRs are used. Where the source of the ITRs is from AAV2 and the AAV capsid is from another AAV source, the resulting vector may be termed pseudotyped. However, other configurations of these elements may be suitable.

Optionally, the PTH constructs described herein may be delivered via viral vectors other than rAAV. Such other viral vectors may include any virus suitable for gene therapy, including but not limited to adenovirus; herpes virus; lentivirus; retrovirus; etc. Suitably, where one of these other vectors is generated, it is produced as a replication-defective viral vector.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

Also provided are compositions which include the viral vector constructs described herein. The pharmaceutical compositions described herein are designed for delivery to subjects in need thereof by any suitable route or a combination of different routes. Direct delivery to the liver (optionally via intravenous, via the hepatic artery, or by transplant), oral, inhalation, intranasal, intratracheal, intraarterial, intraocular, intravenous, intramuscular, subcutaneous, intradermal, and other parental routes of administration. The viral vectors described herein may be delivered in a single composition or multiple compositions. Optionally, two or more different AAV may be delivered, or multiple viruses [see, e.g., WO 2011/126808 and WO 2013/049493]. In another embodiment, multiple viruses may contain different replication-defective viruses (e.g., AAV and adenovirus). In one embodiment, administration is intramuscular. In another embodiment, administration is intravenous.

The replication-defective viruses can be formulated with a physiologically acceptable carrier for use in gene transfer and gene therapy applications. In the case of AAV viral vectors, quantification of the genome copies (“GC”) may be used as the measure of the dose contained in the formulation. Any method known in the art can be used to determine the genome copy (GC) number of the replication-defective virus compositions of the invention. One method for performing AAV GC number titration is as follows: Purified AAV vector samples are first treated with DNase to eliminate un-encapsidated AAV genome DNA or contaminating plasmid DNA from the production process. The nuclease resistant particles are then subjected to heat treatment to release the genome from the capsid. The released genomes are then quantitated by real-time PCR using primer/probe sets targeting specific region of the viral genome (usually poly A signal). Another suitable method for determining genome copies is quantitative-PCR (qPCR), particularly the optimized qPCR or digital droplet PCR [Lock Martin, et al, Human Gene Therapy Methods. April 2014, 25(2): 115-125. doi:10.1089/hgtb.2013.131, published online ahead of editing Dec. 13, 2013].

Also, the replication-defective virus compositions can be formulated in dosage units to contain an amount of replication-defective virus that is in the range of about 1.0×10⁹ GC to about 1.0×10¹⁵ GC. In another embodiment, this amount of viral genome may be delivered in split doses. In one embodiment, the dose is about 1.0×10¹⁰ GC to about 3.0×10¹⁴ GC for an average human subject of about 70 kg. In another embodiment, the dose about 1×10⁹ GC. For example, the dose of AAV virus may be about 1×10¹⁰ GC, 1×10¹¹ GC, about 5×10¹¹ GC, about 1×10¹² GC, about 5×10¹² GC, or about 1×10¹³ GC. In another embodiment, the dosage is about 1.0×10⁹ GC/kg to about 3.0×10¹⁴ GC/kg for a human subject. In another embodiment, the dose about 1×10⁹ GC/kg. For example, the dose of AAV virus may be about 1×10¹⁰ GC/kg, 1×10¹¹ GC/kg, about 5×10¹¹ GC/kg, about 1×10¹² GC/kg, about 5×10¹² GC/kg, or about 1×10¹³ GC/kg. In one embodiment, the constructs may be delivered in volumes from 1 μL to about 100 mL. As used herein, the term “dosage” or “amount” can refer to the total dosage or amount delivered to the subject in the course of treatment, or the dosage or amount delivered in a single unit (or multiple unit or split dosage) administration.

The above-described recombinant vectors may be delivered to host cells according to published methods. The rAAV, preferably suspended in a physiologically compatible carrier, may be administered to a desired subject including a human. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention.

In another embodiment, the composition includes a carrier, diluent, excipient and/or adjuvant. In certain embodiments, for administration to a human patient, the rAAV is suitably suspended in an aqueous solution containing saline, a surfactant, and a pharmaceutically and/or physiologically compatible salt or mixture of salts. Suitably, the formulation is adjusted to a physiologically acceptable pH, e.g., in the range of pH 6 to 9, or pH 6.0 to 7.5, or pH 6.2 to 7.7, or pH 6.5 to 7.5, pH 7.0 to 7.7, or pH 7.2 to 7.8, or about 7.0. In certain embodiments, the formulation is adjusted to a pH of about 6.0, about 6.1, about 6.2, about 6.3, about 6.4, about 6.5, about 6.6, about 6.7, about 6.8, about 6.9, about 7.0, about 7.1, about 7.2, about 7.3 about 7.4, about 7.5, about 7.6, about 7.7, or about 7.8. In certain embodiments, a pH of about 7.28 to about 7.32, about 6.0 to about 7.5, about 6.2 to about 7.7, about 7.5 to about 7.8, about 6.0, about 6.1, about 6.2, about 6.3, about 6.4, about 6.5, about 6.6, about 6.7, about 6.8, about 6.9, about 7.0, about 7.1, about 7.2, about 7.3 about 7.4, about 7.5, about 7.6, about 7.7, or about 7.8 may be desired. In certain embodiments, for intravenous delivery, a pH of about 6.8 to about 7.2 may be desired. However, other pHs within the broadest ranges and these subranges may be selected for other route of delivery.

Optionally, the compositions of the invention may contain, in addition to the rAAV and/or variants and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens. ethyl vanillin, glycerin, phenol, and parachlorophenol. Suitable chemical stabilizers include gelatin and albumin.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells. In particular, the rAAV vector delivered transgenes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

Dosages of the vector depends primarily on factors such as the condition being treated, the age, weight and health of the patient, and may thus vary among patients. For example, a therapeutically effective human dosage of viral vector is generally in the range of from about 25 to about 1000 microliters to about 100 mL of solution containing concentrations of from about 1×10⁹ to 1×10¹⁶ genomes virus vector (to treat an average subject of 70 kg in body weight) including all integers or fractional amounts within the range, and preferably 1.0×10¹² GC to 1.0×10¹³ GC for a human patient. The composition of the invention may be delivered in a volume of from about 0.1 μL to about 10 mL, including all numbers within the range, depending on the size of the area to be treated, the viral titer used, the route of administration, and the desired effect of the method. In one embodiment, the volume is about 50 μL. In another embodiment, the volume is about 70 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 250 μL. In another embodiment, the volume is about 300 μL. In another embodiment, the volume is about 450 μL. In another embodiment, the volume is about 500 μL. In another embodiment, the volume is about 600 μL. In another embodiment, the volume is about 750 μL. In another embodiment, the volume is about 850 μL. In another embodiment, the volume is about 1000 μL. In another embodiment, the volume is about 1.5 mL. In another embodiment, the volume is about 2 mL. In another embodiment, the volume is about 2.5 mL. In another embodiment, the volume is about 3 mL. In another embodiment, the volume is about 3.5 mL. In another embodiment, the volume is about 4 mL. In another embodiment, the volume is about 5 mL. In another embodiment, the volume is about 5.5 mL. In another embodiment, the volume is about 6 mL. In another embodiment, the volume is about 6.5 mL. In another embodiment, the volume is about 7 mL. In another embodiment, the volume is about 8 mL. In another embodiment, the volume is about 8.5 mL. In another embodiment, the volume is about 9 mL. In another embodiment, the volume is about 9.5 mL. In another embodiment, the volume is about 10 mL.

In some embodiments, a concentration of a recombinant adeno-associated virus carrying a nucleic acid sequence encoding the desired transgene under the control of the regulatory sequences desirably ranges from about 10⁷ and 10¹⁴ genome copies per milliliter (GC/mL) in a composition.

In one embodiment, the dosage of rAAV in a composition is from about 1.0×10⁹ GC/kg of body weight to about 1.5×10¹³ GC/kg. In one embodiment, the dosage is about 1.0×10¹⁰ GC/kg. In one embodiment, the dosage is about 1.0×10¹¹ GC/kg. In one embodiment, the dosage is about 1.0×10¹² GC/kg. In one embodiment, the dosage is about 5.0×10¹² GC/kg. In one embodiment, the dosage is about 1.0×10¹³ GC/kg. All ranges described herein are inclusive of the endpoints.

In one embodiment, the effective dosage (total genome copies delivered) is from about 10⁷ to 10¹³ genome copies. In one embodiment, the total dosage is about 10⁸ genome copies. In one embodiment, the total dosage is about 10⁹ genome copies. In one embodiment, the total dosage is about 10¹⁰ genome copies. In one embodiment, the total dosage is about 10¹¹ genome copies. In one embodiment, the total dosage is about 10¹² genome copies. In one embodiment, the total dosage is about 10¹³ genome copies. In one embodiment, the total dosage is about 10¹⁴ genome copies. In one embodiment, the total dosage is about 10¹⁵ genome copies.

It is desirable that the lowest effective concentration of virus be utilized in order to reduce the risk of undesirable effects, such as toxicity. Still other dosages and administration volumes in these ranges may be selected by the attending physician, taking into account the physical state of the subject, preferably human, being treated, the age of the subject, the particular disorder and the degree to which the disorder, if progressive, has developed.

In certain embodiments, the composition comprises an rAAV comprising an inducible PTH agonist construct. In certain embodiments, the inducing agent or molecule is a rapamycin or a rapalog. In certain embodiments, the inducing agent is rapamycin, and is administered at least one or more, at least two or more, at least three or more times following rAAV-comprising composition. In some embodiments the rapamycin is administered at dose at least about 4 to at least about 40 nM. In certain embodiments, the inducing agent (i.e., rapamycin) is administered at a dose at least about 0.1 mg/kg to at least about 3.0 mg/kg. In certain embodiments, the inducing agent (i.e., rapamycin) is administered at a dose at least about 0.5 mg/kg to at least about 2.0 mg/kg.

The viral vectors and other constructs described herein may be used in preparing a medicament for delivering a PTH fusion protein construct to a subject in need thereof, supplying PTH having an increased half-life to a subject, and/or for treating HP in a subject. Thus, in another aspect a method of treating HP is provided. The method includes administering a composition as described herein to a subject in need thereof. In one embodiment, the composition includes a viral vector containing a PTH fusion protein expression cassette, as described herein.

As used herein, the term “treatment” or “treating” is defined encompassing administering to a subject one or more compounds or compositions described herein for the purposes of amelioration of one or more symptoms of short bowel syndrome. “Treatment” can thus include one or more of reducing progression of HP, reducing the severity of the symptoms, removing the disease symptoms, delaying progression of disease, or increasing efficacy of therapy in a given subject.

In another embodiment, a method for treating HP in a subject is provided. The method includes administering a viral vector comprising a nucleic acid molecule comprising a sequence encoding a fusion protein as described herein. In one embodiment, the subject is a human.

A course of treatment may optionally involve repeat administration of the same viral vector (e.g., an AAVrh91 vector) or a different viral vector (e.g., an AAVrh91 and an AAV3B.AR2.12). Still other combinations may be selected using the viral vectors described herein. Optionally, the composition described herein may be combined in a regimen involving nutritional therapy (enteral or parenteral nutrition), medications, such as those used to control stomach acid, reduce diarrhea, or improve intestinal absorption, or a PTH protein, or surgery. In certain embodiments, the AAV vector and the combination therapy are administered essentially simultaneously. In other embodiments, the AAV vector is administered first. In other embodiments, the combination therapy is delivered first.

In one embodiment, the composition is administered in combination with an effective amount of a PTH protein. Various commercially available PTH products are known in the art. including, without limitation, teriparatide (PTH1-34), abaloparatide, and recombinant PTH (rhPTH1-84). In another embodiment, the composition is administered in combination with an effective amount of calcium carbonate or vitamin D.

In some embodiments, combination of the rAAV described herein with PTH protein decreases PTH protein dose requirements in the subject, as compared to prior to treatment with the viral vector. Such dose requirements may be reduced by 10% or more, 20% or more, 30% or more, 40% or more, 50% or more, 60% or more, 70% or more, 80% or more, or 90% or more. The treating physician may determine the correct dosage of PTH protein needed by the subject. For example, the subject may be being treated using PTH protein or other therapy, which the treating physician may continue upon administration of the AAV vector. Such PTH protein or other co-therapy may be continued, reduced, or discontinued as needed subsequently.

In one embodiment, composition comprising the expression cassette, vector genome, rAAV, or other composition described herein for gene therapy is delivered as a single dose per patient. In one embodiment, the subject is delivered a therapeutically effective amount of a composition described herein. As used herein, a “therapeutically effective amount” refers to the amount of the expression cassette or vector, or a combination thereof that delivers and expresses in the target cells an amount of PTH fusion protein sufficient to reach therapeutic goal. The therapeutically effective amount may be selected by the treating physician, or guided based on previously determined guidelines. For example, teriparatide may be provided at an initial dose of 20 μg subcutaneously twice daily. The rAAV may be delivered to the subject and then supplemented with oral or subcutaneous teriparatide, or other medication as needed to reach the equivalent of the desired dosage of 20 μg subcutaneously twice daily.

In certain embodiments, the therapeutic goal is to ameliorate or treat one or more of the symptoms of HP. A therapeutically effective amount may be determined based on an animal model, rather than a human patient.

As used herein when used to refer to vp capsid proteins, the term “heterogenous” or any grammatical variation thereof, refers to a population consisting of elements that are not the same, for example, having vp1, vp2 or vp3 monomers (proteins) with different modified amino acid sequences. SEQ ID NO: 38 provides the encoded amino acid sequence of the AAVrh91 vp1 protein. The term “heterogenous” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine-glycine pairs and optionally further comprising other deamidated amino acids, wherein the deamidation results in an amino acid change and other optional modifications.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins is at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine-glycine pairs.

As used herein, a “stock” of rAAV refers to a population of rAAV. Despite heterogeneity in their capsid proteins due to deamidation, rAAV in a stock are expected to 5 share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected. As used herein the terms “PTH construct”, “PTH expression construct” and synonyms include the PTH sequence as described herein in combination with a leader and fusion domain. The terms “PTH construct”. “PTH expression construct” and synonyms can be used to refer to the nucleic acid sequences encoding the PTH fusion protein or the expression products thereof.

The terms “percent (%) identity”, “sequence identity”. “percent sequence identity”, or “percent identical” in the context of nucleic acid sequences refers to the bases in the two sequences which are the same when aligned for correspondence. The length of sequence identity comparison may be over the full-length of the genome, the full-length of a gene coding sequence, or a fragment of at least about 100 to 150 nucleotides, or as desired. However, identity among smaller fragments, e.g., of at least about nine nucleotides, usually at least about 20 to 24 nucleotides, at least about 28 to 32 nucleotides, at least about 36 or more nucleotides, may also be desired. Multiple sequence alignment programs are also available for nucleic acid sequences. Examples of such programs include, “Clustal W”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference.

By the term “highly conserved” is meant at least 80% identity, preferably at least 90% identity, and more preferably, over 97% identity. Identity is readily determined by one of skill in the art by resort to algorithms and computer programs known by those of skill in the art.

Unless otherwise specified by an upper range, it will be understood that a percentage of identity is a minimum level of identity and encompasses all higher levels of identity up to 100% identity to the reference sequence. Unless otherwise specified, it will be understood that a percentage of identity is a minimum level of identity and encompasses all higher levels of identity up to 100% identity to the reference sequence. For example, “95% identity” and “at least 95% identity” may be used interchangeably and include 95%, 96%, 97%, 98%, 99%, and up to 100% identity to the referenced sequence, and all fractions therebetween.

The terms “percent (%) identity”, “sequence identity”, “percent sequence identity”, or “percent identical” in the context of amino acid sequences refers to the residues in the two sequences which are the same when aligned for correspondence. Percent identity may be readily determined for amino acid sequences over the full-length of a protein, polypeptide, about 70 amino acids to about 100 amino acids, or a peptide fragment thereof or the corresponding nucleic acid sequence coding sequencers. A suitable amino acid fragment may be at least about 8 amino acids in length, and may be up to about 150 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. Alignments are performed using any of a variety of publicly or commercially available Multiple Sequence Alignment Programs. Sequence alignment programs are available for amino acid sequences, e.g., the “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

It is to be noted that the term “a” or “an” refers to one or more. As such, the terms “a” (or “an”). “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

“Patient” or “subject” as used herein means a mammalian animal, including a human. a veterinary or farm animal, a domestic animal or pet, and animals normally used for clinical research. In one embodiment, the subject of these methods and compositions is a human. In another embodiment, the subject is not a feline.

As used herein, the term “about” means a variability of 10% (±10%, e.g., ±1, ±2, ±3, ±4, ±5, ±6, ±7, ±8, ±9, ±10, or values therebetween) from the reference given, unless otherwise specified.

In certain instances, the term “E+ #” or the term “e+ #” is used to reference an exponent. For example, “5E10” or “5e10” is 5×10¹⁰. These terms may be used interchangeably.

The term “regulation” or variations thereof as used herein refers to the ability of a composition to inhibit one or more components of a biological pathway.

As used herein, “disease”, “disorder” and “condition” are used interchangeably, to indicate an abnormal state in a subject.

Unless defined otherwise in this specification, technical and scientific terms used herein have the same meaning as commonly understood by one of ordinary skill in the art and by reference to published texts, which provide one skilled in the art with a general guide to many of the terms used in the present application.

A reference to “one embodiment” or “another embodiment” in describing an embodiment does not imply that the referenced embodiment is mutually exclusive with another embodiment (e.g., an embodiment described before the referenced embodiment).

unless expressly specified otherwise.

### EXAMPLES

The following examples are provided to illustrate various embodiments of the present invention. The EXAMPLES are not intended to limit the present invention in any way.

### Example 1—Construction of PTH Vectors

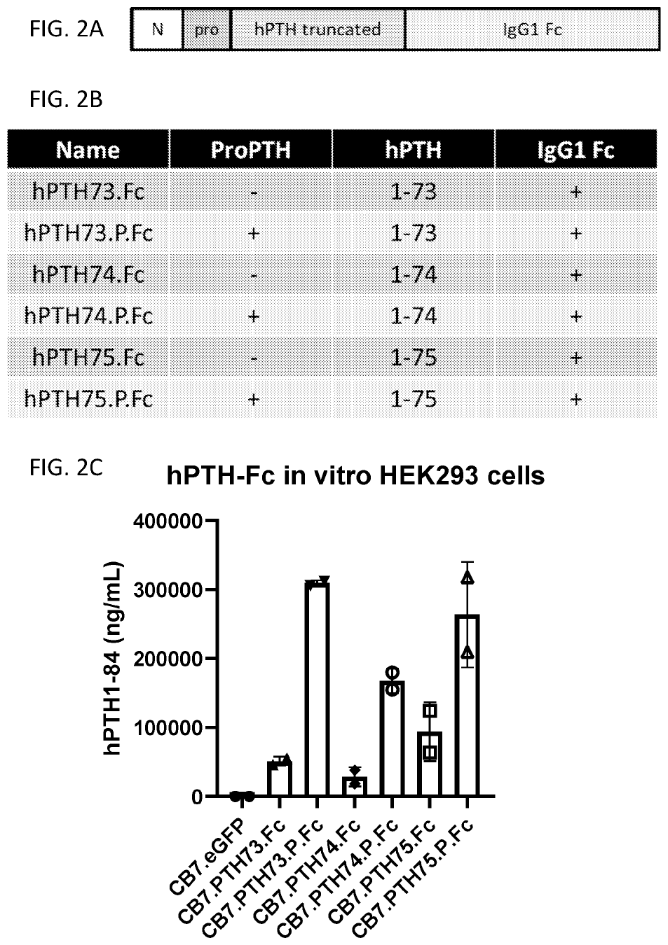

We have reported a pharmacologically regulated gene expression system with which transgene expression can be controlled by orally available drugs such as rapamycin and its proteins (Rapalogs). This gene circuit can be delivered to skeletal muscle by AAV vectors and provide persistent regulated transgene expression for more than 5 years in nonhuman primates (Rivera et al., 2005). Applying this technology to PTH can be a solution for HP patients to avoid daily injections of PTH protein and be safe in case patients need the treatment. We developed a variety of versions of PTH transgene for preferable kinetics of PTH expression upon rapamycin with interests in the level and duration of induced expression to make potent AAV vector that can provide therapeutic level of PTH expression with less vector doses and with less frequent oral inducers. FIG. 2B.

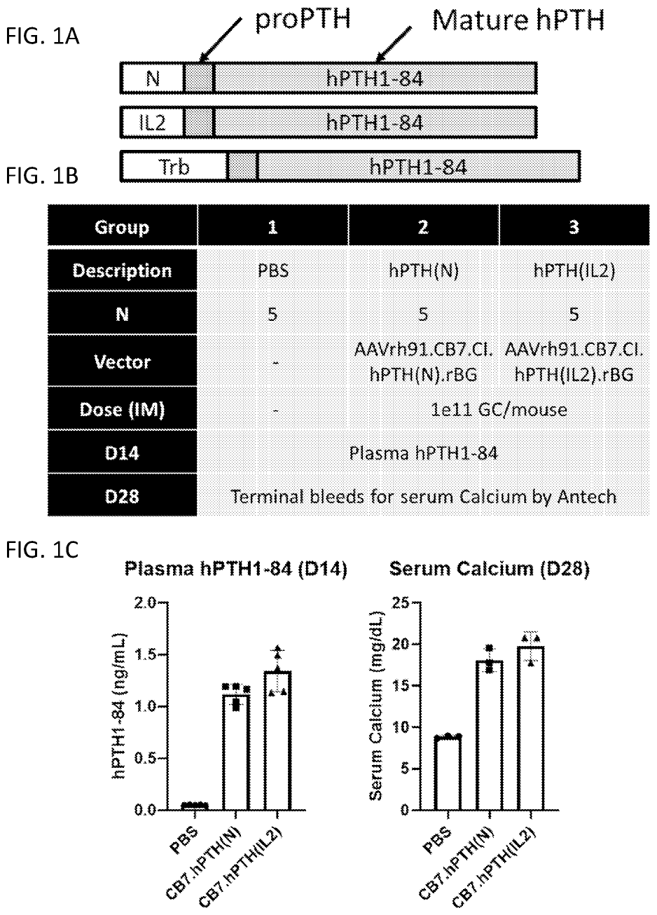

All transgenes were codon optimized towards general human codon usage. A variety of secretory sequences were also introduced for better expression by skeletal muscle cells. FIG. 1A. For longer serum half-life, several N-terminal truncated versions of PTH were fused with Fc domains from human IgG1 and IgG4 or serum albumin. FIG. 2A.

More specifically, vectors were constructed in which a leader sequence was placed upstream of one of several PTH amino acid sequences followed by a fusion domain. FIG. 2B. The sequences were produced, and cloned into an expression vector containing a CMV promoter under the control of an inducible expression system. The expression construct was flanked by AAV2 ITRs. In other construct, the sequences were produced, and cloned into an expression vector containing a CB7 constitutive promoter.

The following constructs were packaged in an AAVrh91 vector by triple transfection and iodixanol gradient purification, as previously described.

### Example 2—In Vitro Expression

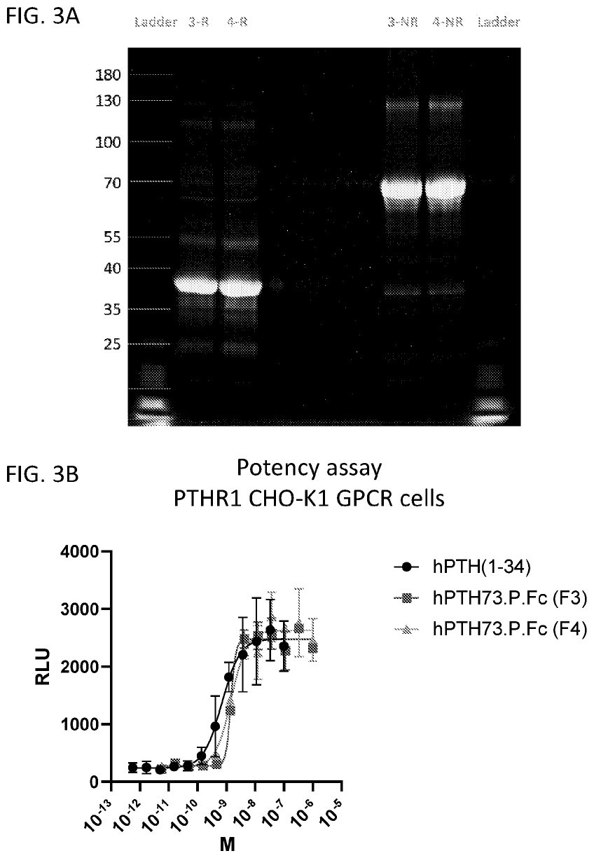

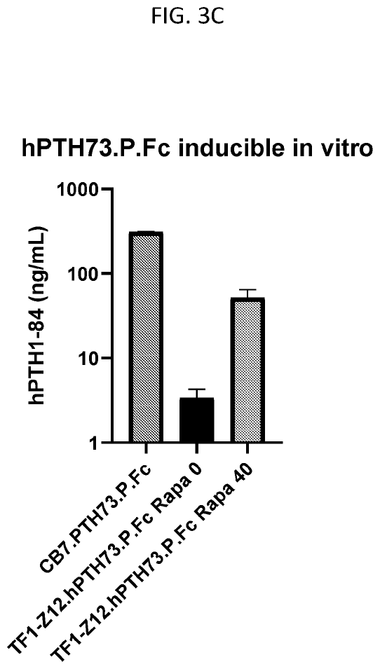

PTH fusions were measured in culture supernatants of HEK293 cells transfected with plasmids for inducible human PTH-Fc. PTH-Fc was quantified by staining as shown in FIG. 3A. Fractions 3 and 4, under reduced (R) and non-reduced conditions (NR) are shown. PTH is unable to form a dimer under reduced conditions. The potency of these purified proteins was tested. The two tested fusions were found to have similar potency to PTH(1-34). FIG. 3B Inducible constructs were tested with 0 or 40 nM rapamycin. The inducible construct expressed at slightly lower amounts as compared to the constitutive construct. (FIG. 3C).

### Example 3—Pilot Expression in Rag1KO Mice

As shown in FIG. 1B, Rag1KO female mice were treated with an injection of the vector AAVrh91.CB7.CI.hPTH(N).rBG (1×10¹¹ GC/mouse) (with native leader) or AAVrh91.CB7.CI.hPTH(IL2).rBG (1×10¹¹ GC/mouse) (with IL2 leader) via IM route of administration. Plasma at day 14 showed similar PTH levels, with the IL2 leader providing slightly higher secreted PTH. FIG. 1C. Serum calcium at day 28 showed similar results. FIG. 1C.

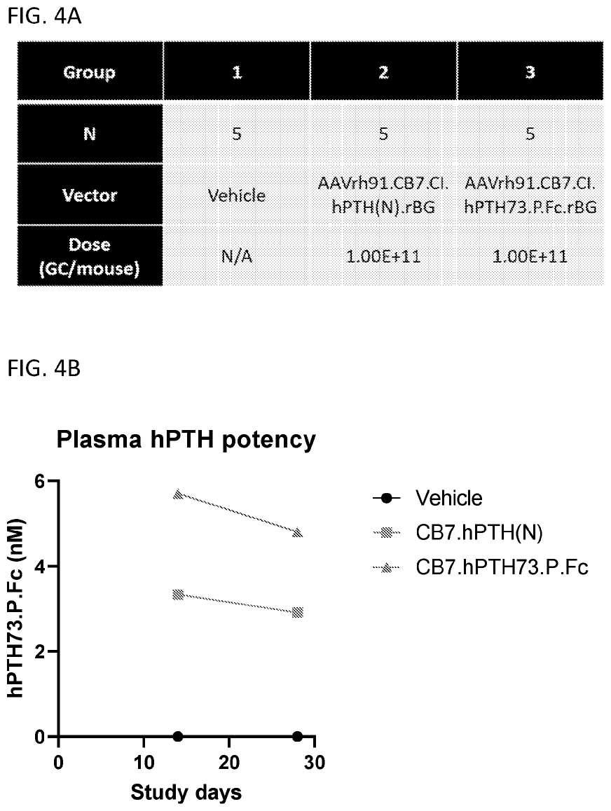

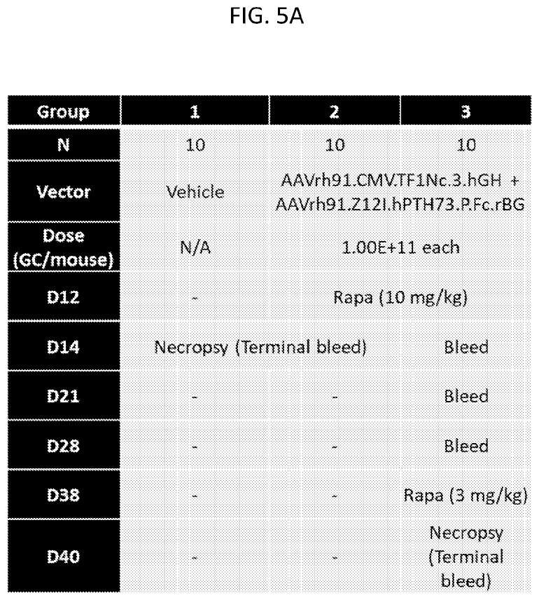

Rag1KO female mice were treated with an injection of the vector AAVrh91.CB7.CI.hPTH(N).rBG (1×10¹¹ GC/mouse) or AAVrh91.CB7.CI.hPTH73.P.Fc.rBG (1×10¹¹ GC/mouse) via IM route of administration. FIG. 4A. Plasma was isolated at days 14 and 28 and tested for hPTH potency. CB7.hPTH73.P.Fc showed superior potency as compared to the construct lacking a fusion. FIG. 4B.

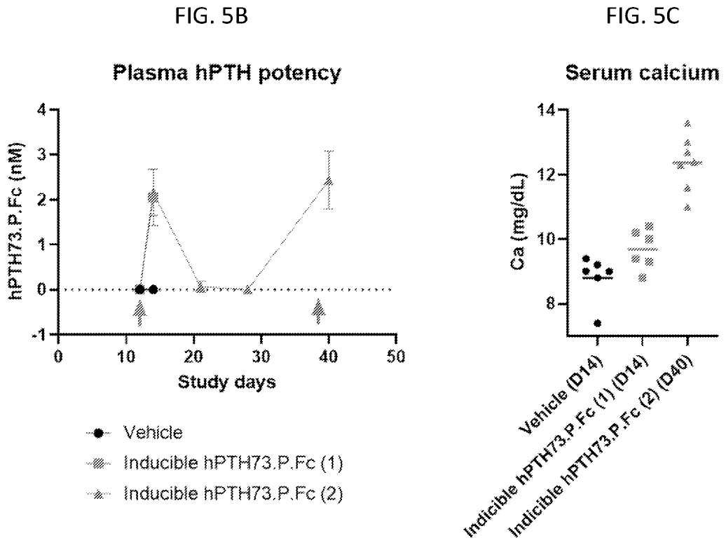

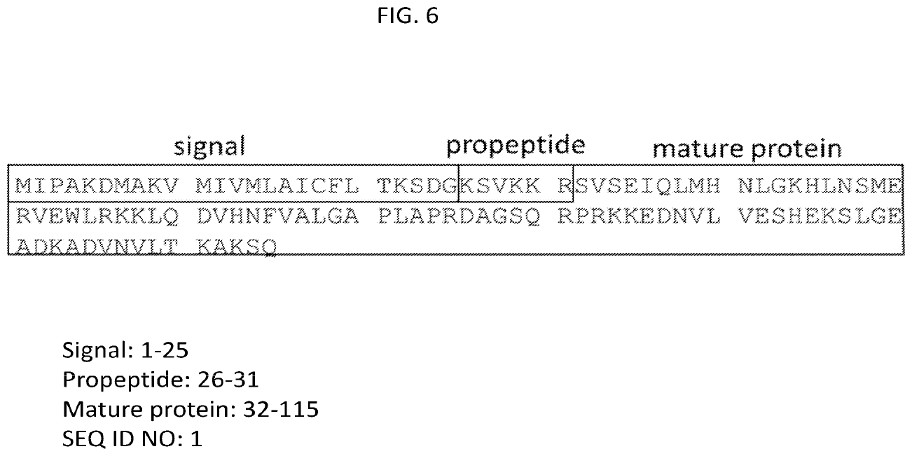

As shown in FIG. 5A, Rag1KO female mice were treated with an injection of the vectors AAVrh91.CMV.TF1Nc.3.hGH (1×10¹¹ GC/mouse) and AAVrh91.CB7.CI.hPTH73.P.Fc.rBG (1×10¹¹ GC/mouse) via IM route of administration. Rapamycin at a dosage of 10 mg/kg was administered on day 12 (for group 2-3), and 3 mg/kg on day 38 (for group 3). Groups 1 and 2 were bled and necropsied on day 14, with group 3 being bled on days 14, 21, 28, and 40 (necropsy). Plasma was isolated and tested for potency (FIG. 5B). Groups 2 and 3 showed comparable potency on day 14, with the second rapamycin induction producing an increase in potency for group 3. Serum calcium is shown in FIG. 5C.

### Example 4—Constitutive PTH-Fc Vector in AFG Monkeys

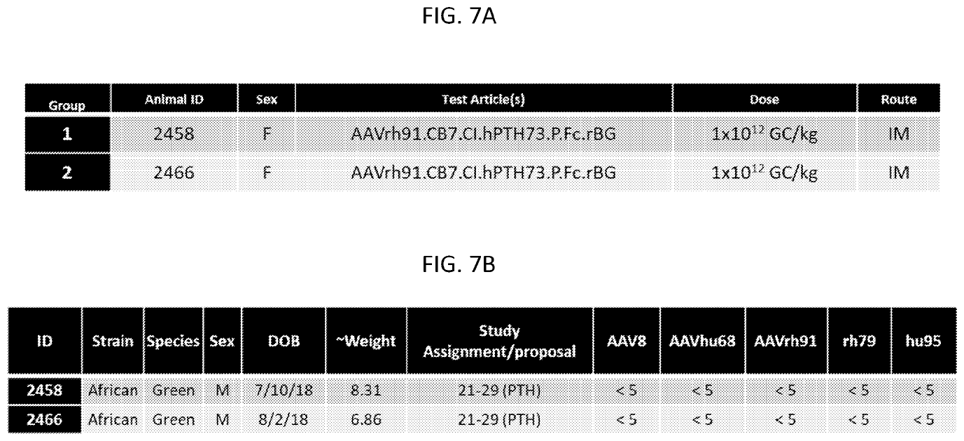

As shown in FIG. 7, female African green monkeys were treated with an injection of the vector AAVrh91.CB7.CI.hPTH73.P.Fc.rBG (1×10¹² GC/kg) via IM route of administration. These monkeys showed NAB titers as shown in FIG. 7B.

### Example 5—TPTX model in Rag2KO Rats

Rag2KO rats were Thyroparathyroidectomized (TPTX) to validate the TPTX model. 8 week old Sprague Dawley Rat/Rag2 KO rat subjected to surgical excision of parathyroid gland using 5-aminolevulinic acid fluorescent identification technique. 5-ALA. 500 mg/kg is administered i.p, prior to surgery, leading to phototoxicity. Thus rats are placed under subdued light for 2 hours prior to surgery. Midline vertical incision is made in neck, and red fluorescent PTH glands are observed under illumination with white-blue light and UV filter. Glands are excised with cold knife. Post-op complications are low calcium levels, leading to uncontrolled muscle spasms and contractions. Death occurs within 4 days without supplemental calcium, which is provided in water or custom formulated diet (AIN-93G 0.5% Ca).

TPTX Rats were injected IM with vehicle, inducible.hPTH73.P.Fc (1×10¹² GC/kg or 1×10¹³ GC/kg). First rapamycin dose of 10 mg/kg i.p, was given at day 14, and 2^{nd} dose (3 mg/kg i.p.) at day 40. Clinical signs are shown in the table below.

| | | | | | Died or | |
|---|---|---|---|---|---|---|
| Group | ID | Day 0 | Day 14 | Day 40 | Euthanized | Clinical signs |
| 1 | 8001 | Vehicle | 1st | 2nd | | |
| | 8002 | | Rapamycin | Rapamycin | | |
| | 8003 | | 10 mg/kg | 3 mg/kg | | |
| 2 | 8007 | Ind. | (ip) | (ip) | 60 | hunched, ruffled, |
| | | hPTH73.P.Fc | | | | and dehydrated; |
| | | 1e12 GC/kg | | | | continued to |
| | | each | | | | decline |
| | 8008 | | | | 68 | weight loss, |
| | | | | | | lethargy, and |
| | | | | | | hindlimb paresis |
| | | | | | | vs. lameness |
| | 8009 | | | | 18 | hunched, ruffled, |
| | | | | | | and quiet; |
| | | | | | | profound hindlimb |
| | | | | | | lameness |
| | 8010 | | | | 17 | an acute non- |
| | | | | | | weight bearing left |
| | | | | | | hindlimb lameness |
| | | | | | | (FD on Aug. 28, 2022) |
| 3 | 8004 | Ind. | | | 12 | hunched, ruffled, |
| | | hPTH73.P.Fc | | | | and dehydrated; |
| | | 1e13 GC/kg | | | | continued to |
| | | each | | | | decline, and she |
| | | | | | | began having |
| | | | | | | uncontrolled |
| | | | | | | muscle |
| | | | | | | fasciculations vs. |
| | | | | | | seizure activity |
| | 8005 | | | | 53 | hunched, ruffled, |
| | | | | | | and dehydrated; |
| | | | | | | continued to |
| | | | | | | decline |
| | 8006 | | | | 25 | hunched, ruffled, |
| | | | | | | and dehydrated; |
| | | | | | | continued to |
| | | | | | | decline |

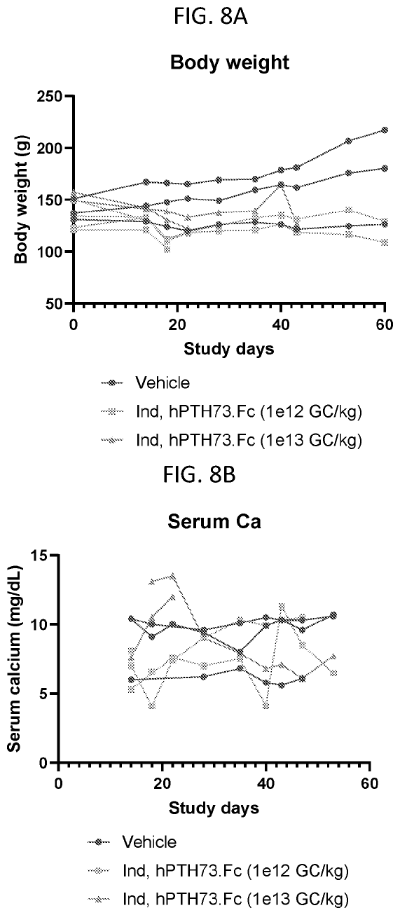

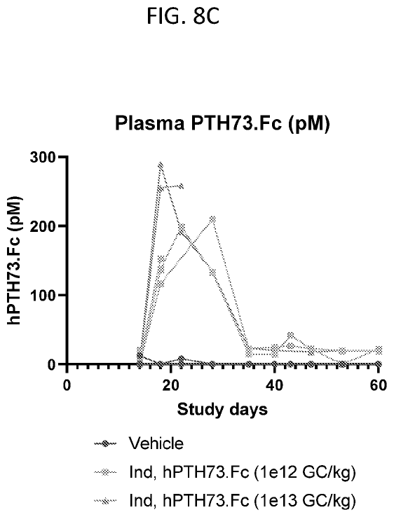

Results are shown in FIGS. 8A-8C. Two rats with vehicle injections had incomplete parathyroid removal. The rats showed severe decline of condition from an unknown cause.

### Example 6—Constitutive PTH-Fc Vector in Rag2KO Rats

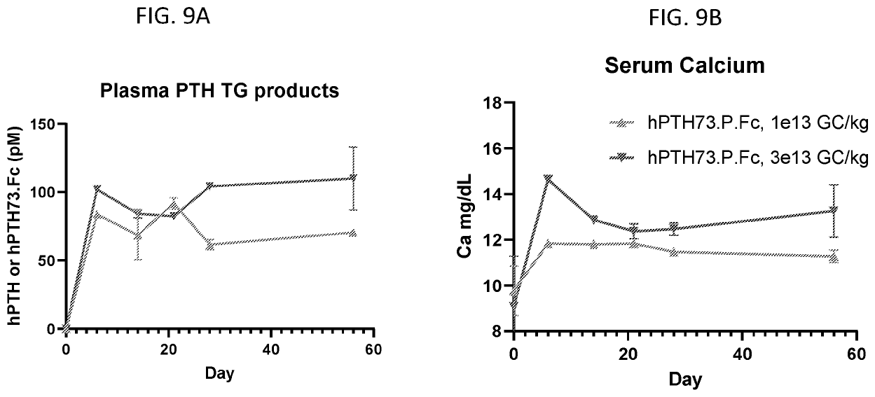

Rag2KO rats (3 rats/group) were injected IM with AAVrh91.CB7.CI.hPTH73.P.Fc.rBG (1×10¹³ GC/kg or 3×10¹³ GC/kg). One rat from the group receiving 3×10¹³ GC/kg showed hindlimb paralysis and had to be euthanized possibly due to pathologic fracture of spinal cord. Plasma PTH (FIG. 9A) and serum calcium (FIG. 9B) were measured.

All documents cited in this specification, are incorporated herein by reference. U.S. Provisional Patent Application No. 63/316,215, filed Mar. 3, 2022, is incorporated herein by reference. While the invention has been described with reference to particular embodiments, it will be appreciated that modifications can be made without departing from the spirit of the invention. Such modifications are intended to fall within the scope of the appended claims.

1. A composition comprising a nucleic acid comprising a sequence encoding a fusion protein comprising a parathyroid hormone (PTH) protein and an IgG4 Fc, wherein the fusion protein has the sequence of SEQ ID NO: 1, 11, 13, 15, 17, 19, 21, 23, 25, 27, 29, 39, 42, 44, 46, or 48, or a sequence at least 99% identical thereto.

2. The composition according to claim 1, wherein the sequence encoding the fusion protein is SEQ ID NO: 12, 14, 16, 18, 20, 22, 24, 26, 28, or 30, or a sequence sharing at least 75% identical thereto.

3. The composition comprising a viral vector comprising:
(a) an adeno-associated virus (AAV) capsid, and
(b) a vector genome packaged in the AAV capsid, said vector genome comprising AAV inverted terminal repeats (ITRs), the coding sequence for the fusion protein, and regulatory sequences that direct expression of the fusion protein.

4. The composition according to claim 3, wherein the viral vector is a recombinant AAV (rAAV) having the AAV capsid of AAVrh91 or AAVhu68.

5. The composition according to claim 1, wherein the fusion protein is under the control of an inducible gene expression system comprising a regulatable promoter, an activation domain, and a DNA binding domain.

6. (canceled)

7. The composition according to claim 3, wherein the AAV inverted terminal repeats (ITRs) are an AAV2 5′ ITR and an AAV2 3′ ITR which flank the fusion protein coding sequence and regulatory sequences.

8. The composition according to claim 3, wherein the vector genome comprises a CB7 promoter and a rabbit globin poly A.

9. The composition according to claim 5, wherein the inducible gene expression system comprises
(a) an activation domain comprising a transactivation domain and a FKBP12-rapamycin binding (FRB) domain of FKBP12-rapamycin-associated protein (FRAP);
(b) a DNA binding domain comprising a zinc finger homeodomain (ZFHD) and one, two or three FK506 binding protein domain (FKBP) subunit genes; and
(c) at least one copy of the binding site for ZFHD followed by a minimal promoter, and
(d) a regulatable promoter.

10. The composition according to claim 9, wherein the inducible gene expression system is comprised in one vector.

11. The composition according to claim 9, wherein the inducible gene expression system is comprised in two vectors.

12. The composition according to claim 9, wherein the transactivation domain comprises a portion of NF-κB p65.

13. The composition according to claim 9, wherein the regulatable promoter is a constitutive promoter.

14. The composition according to claim 12, wherein the regulatable promoter is a CMV promoter.

15. The composition according to claim 9, further comprising an IRES or 2A.

16. The composition according to claim 9, comprising at least 8 copies of the binding site for ZFHD.

17. A composition according to claim 5 comprising a regulatable promoter; an activation domain comprising a p65 transactivation domain and a FKBP12-rapamycin binding (FRB) domain of FKBP12-rapamycin-associated protein (FRAP); a DNA binding domain comprising a zinc finger homeodomain (ZFHD) and three FK506 binding protein domain (FKBP) subunit genes; 12 copies of the binding site for ZFHD, and a sequence encoding a fusion protein comprising a PTH protein of any of SEQ ID NO: 1, 11, 13, 15, 17, 19, 21, 23, 25, 27, 29, 39, 42, 44, 46, or 48, and regulatory sequences which direct expression of the fusion protein.

18-20. (canceled)

21. The composition according to claim 1, wherein the composition is formulated to be administered a dose of 1×10⁹ GC/kg to 5×10¹³ GC/kg or rAAV 1×10¹⁰ to 1.5×10¹⁵ GC of the rAAV.

22. (canceled)

23. The composition according to claim 1, wherein the rAAV is delivered intramuscularly or intravenously.

24. A method of treating a subject having hypoparathyroidism, comprising delivering to the subject an rAAV having an AAV capsid from AAVrh91 or AAVhu68, and a vector genome packaged in the AAV capsid, said vector genome comprising AAV inverted terminal repeats (ITRs), a sequence encoding a fusion protein comprising a PTH protein of any of SEQ ID NO: 1, 11, 13, 15, 17, 19, 21, 23, 25, 27, 29, 39, 42, 44, 46, or 48, and regulatory sequences which direct expression of the fusion protein.

25-28. (canceled)
